(19) United States
(12) Patent Application Publication
(10) Pub. No.: US 2017/0347891 A1
(43) Pub. Date: Dec. 7, 2017

(54) Thermal Transport Characteristics of Human Skin Measured In Vivo Using Thermal Elements

(71) Applicants: THE BOARD OF TRUSTEES OF THE UNIVERSITY OF ILLINOIS, Uubana, IL (US); L'OREAL, Paris (FR)

(72) Inventors: John A. ROGERS, Champaign, IL (US); Richard Chad WEBB, Saint Paul, MI (US); Siddharth KRISHNAN, Urbana, IL (US); Guive BALOOCH, Clark, NJ (US); Rafal M. PIELAK, Richmond, CA (US)

(21) Appl. No.: 15/515,494

(22) PCT Filed: Oct. 1, 2015
(86) PCT No.: PCT/US15/53452

§ 371 Date: Mar. 29, 2017

Related U.S. Application Data
(60) Provisional application No. 62/058,547, filed on Oct. 1, 2014.

(51) Int. Cl. A61B 5/01; A61B 5/053; A61B 5/00

(52) U.S. Cl. CPC A61B 5/01; A61B 5/4875; A61B 5/0531; A61B 5/441; A61B 5/4848

(57) ABSTRACT

Devices and methods useful for sensing epidermal tissue are disclosed. Thermal data from the devices allows for determination of thermal transport properties, such as thermal conductivity, thermal diffusivity and heat capacity per unit volume. From these data, tissue parameters, such as hydration state, stratum corneum thickness, epidermis thickness and vasculature structure may be determined. These parameters may be used, for example, to evaluate the efficacy of dermatological compounds.

### CROSS-REFERENCE TO RELATED APPLICATIONS

This application claims the benefit of priority from U.S. Provisional Patent Application No. 62/058,547, filed Oct. 1, 2014, which is hereby incorporated by reference in its entirety.

### STATEMENT REGARDING FEDERALLY SPONSORED RESEARCH OR DEVELOPMENT

This invention was made with government support under DGE-1144245 awarded by the National Science Foundation. The government has certain rights in the invention.

### BACKGROUND

Skin is the largest organ of the human body and it provides one of the most diverse sets of functions. The outermost layer, the stratum corneum (SC), serves as a protective barrier and the first defense against physical, chemical and biological damage. The skin also receives and processes multiple sensory stimuli, such as touch, pain and temperature, and aids in the control of body temperature and the flow of fluids in and out of the body. These processes are highly regulated by nervous and circulatory systems, but also depend directly and indirectly on thermal characteristics of the skin.

Measurements of the thermal transport properties of the skin can reveal changes in physical and chemical states of relevance to dermatological health, skin structure and activity, thermoregulation and other aspects of human physiology. Existing methods for in vivo evaluations demand complex systems for laser heating and infrared thermography, or they require rigid, invasive probes. Neither can apply to arbitrary regions of the body, offers modes for rapid spatial mapping, or enables continuous monitoring outside of laboratory settings.

It will be appreciated from the foregoing that epidermal systems are needed for accurate, non-invasive, in vivo skin monitoring. Such epidermal systems would preferably be less complex than existing systems and useable outside laboratory or clinical settings.

### SUMMARY OF THE INVENTION

Devices and methods useful for sensing epidermal tissue are disclosed. Thermal data from the devices allows for determination of thermal transport properties, such as thermal conductivity, thermal diffusivity and heat capacity per unit volume. From these data, tissue parameters, such as hydration state, stratum corneum thickness, epidermis thickness and vasculature structure may be determined. These parameters may be studied as a function of tissue depth or tissue type to provide three-dimensional tissue thermal information.

More advanced multimodal devices and methods may integrate electrical, optical and/or acoustic capabilities in order to provide the unprecedented ability to make simultaneous, independent measurements on the same patient, on the same body location and essentially at the same time, which reduces measurement error.

In an aspect, the present invention is a method of sensing an epidermal tissue of a subject, the method comprising: thermally actuating an epidermal tissue region with one or more thermal elements by delivering a heating power selected from the range of 0.0001 mJ s⁻¹ to 1000 mJ s⁻¹ for a period selected from the range of 10 ms to 1000 s; detecting one or more temperatures of the epidermal tissue proximate to the tissue region with the one or more thermal elements; and generating a depth profile thermal measurement. In some embodiments, the heating power is selected from the range of 0.001 mJ s⁻¹ to 100 mJ s⁻¹, or 0.01 mJ s⁻¹ to 10 mJ s⁻¹, or 0.1 mJ s⁻¹ to 1 mJ s⁻¹. In some embodiments, the heating power is provided for a period selected from the range of 100 ms to 100 s, or 1 s to 50 s.

In some embodiments, the step of generating comprises analyzing the one or more temperatures of the epidermal tissue to provide the depth profile thermal measurement. For example, in an embodiment, the depth profile thermal measurement is thermal conductivity, thermal diffusivity or heat capacity as a function of three-dimensional tissue location.

In an embodiment, the step of generating the depth profile thermal measurement comprises varying the thermal actuation to provide a multifocal response. For example, the multifocal response may be obtained by varying thermal heating power or duration.

In an embodiment, the depth profile thermal measurement extends from a surface of the epidermal tissue to a depth of 4 mm, or from a depth of 250 μm to 4 mm. For other applications, such as non-thermal applications, a depth profile measurement may extend from a surface of the epidermal tissue to a depth of 4 mm, or from a depth of 20 μm to a depth of 4 mm.

In some embodiments, a depth profile thermal measurement is used to determine a three-dimensional hydration profile of tissue. In other embodiments, a depth profile thermal measurement is used to determine a three-dimensional circulation profile of tissue.

Exposure of epidermal tissue to heat has been shown to increase skin permeability. (See, e.g., Park et al., Int. J. Pharm., 2008 Jul. 9; 359(1-2): 94-103.) Accordingly, in some embodiments, the step of thermally actuating increases permeability of the epidermal tissue or at least the stratum corneum. In this way, the step of thermally actuating may increase permeation of active compounds or pharmaceuticals into the epidermal tissue. (Arora, Anubhav, Mark R. Prausnitz, and Samir Mitragotri. “Micro-scale devices for transdermal drug delivery.” International Journal of pharmaceutics 364.2 (2008): 227-236; Prausnitz, Mark R. and Robert Langer. “Transdermal drug delivery.” Nature biotechnology 26.11 (2008): 1261-1268.)

In some embodiments, methods disclosed herein further comprise electrically actuating the epidermal tissue region with a first electrode and obtaining an electrical signal from a second epidermal tissue region with a second electrode. In some embodiments, the first electrode and the second electrode are separated by a distance selected from the range of 50 μm to 10 mm, or 100 μm to 1 mm. In some embodiments, a depth profile extends from a surface of the epidermal tissue to a depth equal to half the separation distance between the first electrode and the second electrode. In an embodiment, the first electrode delivers alternating current having a frequency of 1 kHz to 100 KHz.

In some embodiments, a hydration level or profile of tissue may be used to determine total body hydration. In some embodiments, electrical actuation of the epidermal tissue may be used to determine total body hydration. (Powers et. al. Rapid Measurement of Total Body Water to Facilitate Clinical Decision Making in Hospitalized Elderly Patients. The Journals of Gerontology Series A: Biological Sciences and Medical Sciences; Armstrong et al., Bioimpedance spectroscopy technique: intra-, extracellular, and total body water. Med Sci Sports Exerc 29:1657-1663, 1997; Ritz P.: Bioelectrical impedance analysis estimation of water compartments in elderly diseased patients: the source study. J Gerontol. 56:M344-M348, 2001; Armstrong, L. E. Assessing hydration status: the elusive gold standard. Journal of the American College of Nutrition 26.sup5 (2007): 575S-584S.)

In some embodiments, the one or more thermal elements are provided in conformal contact with the tissue, thereby providing the one or more thermal elements in thermal contact with the epidermal tissue.

In some embodiments, detecting one or more temperatures of the epidermal tissue proximate to the tissue region comprises measuring a distribution of the temperatures of the surface of the epidermal tissue in response to the thermally actuating step.

In some embodiments, detecting one or more temperatures of the epidermal tissue proximate to the tissue region comprises spatio temporally mapping the temperatures of the surface of the epidermal tissue in response to the thermally actuating step.

Certain parameters for actuating and sensing an epidermal tissue of a subject may be selected to facilitate acquisition of specific tissue data. Exemplary parameters are provided in Table 1.

TABLE 1
Thermal Actuator/Sensor Parameters

| | | Actuating | Operational | Measurement |
|---|---|---|---|---|
| Parameter | Mode of Operation | Power | Time Scale | depth |
| Epidermal | DC | 1-10 | 2s- 8 hrs | Can control |
| Thermal | | mW/mm² | | actuation time |
| Conductivity | AC: Pulsed Frequencies from | | | to get |
| | 0.001 Hz to 10 Hz. Facilities | | | measurement |
| | easier rejection of noise, | | | depth from |
| | through Fourier filtering. | | | 250 μm |
| | a. Single | | | (Stratum |
| | Sensor/Actuator: Use | | | Corneum + a |
| | transient temperature | | | part of the |
| | vs. time relationship for | | | epidermis) to 4 |
| | single sensor/actuator. | | | mm (Stratum |
| | Get thermal conductivity | | | Corneum + |
| | at location of each | | | Epidermis + |
| | sensor/actuator by using | | | Dermis), |
| | the mathematical | | | according to: |
| | relationship, | | | Δₚ = \sqrt{αtₘₐₓ}, |
| | ${T}_{measured} = {T}_{∞} + {A}_{1} \frac{Q}{2 π {A}_{2} {k}_{skin}} erfc ( \frac{{A}_{2} r ( t )}{2 \sqrt{α t}} ) ,$ | | | where α is the thermal diffusivity of |
| | where T_{measured} is the | | | the skin and |
| | temperature measured | | | tₘₐₓ is the |
| | by a sensor/actuator | | | measurement |
| | T∞ is the initial | | | depth. |
| | temperature before | | | |
| | heating, erfc is the | | | |
| | complementary error | | | |
| | function kₛₖᵢₙ is the | | | |
| | thermal conductivity of | | | |
| | the skin, αₛₖᵢₙ is the | | | |
| | thermal diffusivity of the | | | |
| | skin, Q is the heating | | | |
| | power, A₂ accounts for | | | |
| | spatial averaging effects | | | |
| | over the sensor/actuator | | | |
| | and A₁ is a calibration | | | |
| | constant accounting for | | | |
| | the device geometry. | | | |
| | b. Single/multiple | | | |
| | actuator, multiple | | | |
| | sensors: Get directional | | | |
| | anisotropies in thermal | | | |
| | conductivity, by using | | | |
| | multiple, strategically | | | |
| | placed sensors around | | | |
| | thew actuator. Can get | | | |
| | thermal conductivity and | | | |
| | diffusivity of the skin | | | |
| | according to the relation: | | | |
| | ${T}_{measured} = {T}_{∞} + {A}_{1} \frac{Q}{2 π r ( t ) {k}_{skin}} erfc ( \frac{r ( t )}{2 \sqrt{α t}} ) ,$ | | | |
| | where T_{measured} is the | | | |
| | temperature measured | | | |
| | by sensor at a | | | |
| | distance r(t) away from | | | |
| | the actuator, T∞ is the | | | |
| | initial temperature | | | |
| | before heating, erfc is | | | |
| | the complementary error | | | |
| | function kₛₖᵢₙ is the | | | |
| | thermal conductivity of | | | |
| | the skin, αₛₖᵢₙ is the | | | |
| | thermal diffusivity of the | | | |
| | skin, Q is the heating | | | |
| | power and A₁ is a | | | |
| | calibration constant | | | |
| | accounting for the | | | |
| | device geometry. | | | |
| Epidermal | DC | 1-10 | 2s- 8 hrs | |
| Thermal | | mW/mm² | | |
| Diffusivity | AC: Pulsed Frequencies from | | | |
| | 0.001 Hz to 10 Hz | | | |
| | Single Sensor/Actuator: Use | | | |
| | transient temperature vs. time | | | |
| | relationship for single | | | |
| | sensor/actuator. Same as | | | |
| | above. | | | |
| | Single actuator, multiple | | | |
| | sensors: Get directional | | | |
| | anisotropies in thermal | | | |
| | conductivity. Same as above. | | | |
| Tissue | Linear relationship, parameters | | | |
| Hydration | of which can be established by | | | |
| | calibrating thermal conductivity | | | |
| | or diffusivity with known | | | |
| | hydration level. Measurements | | | |
| | can be made at all locations on | | | |
| | epidermis. | | | |
| Tissue | Relationship with thickness of | | | |
| Thickness | stratum corneum can be | | | |
| | established by calibrating | | | |
| | against a depth profile tool such | | | |
| | as optical coherence | | | |
| | tomography. Examples of past | | | |
| | locations include the cheek, | | | |
| | heel, plam, dorsal forearm, | | | |
| | volar forearm and the volar wrist | | | |
| | [2]. | | | |

In some embodiments, the one or more thermal elements are individually or separately thermal actuators and sensors.

In some embodiments, the step of delivering a heating power comprises delivering heating power selected from the range of 1 mW mm⁻² to 10 mW mm⁻². In some embodiments, the step of delivering a heating power comprises delivering heating power for a duration of 2 seconds to 8 hours, or 2 seconds to 1 hour, or 2 seconds to 60 seconds. In some embodiments, heating power is delivered over an area of the tissue selected from the range of 0.0001 mm² to 1 cm², or selected from the range of 0.001 mm² to 1 cm², or selected from the range of 0.01 mm² to 1 cm².

In some embodiments, the step of thermally actuating comprises applying a continuous heating power to the epidermal tissue. In other embodiments, the step of thermally actuating comprises applying a pulsed heating power to the epidermal tissue. For example, the pulsed power may have a frequency between 0.001 Hz and 10 Hz with a duty cycle between 0.001% and 100% duty cycle, or the pulsed power may have a frequency between 0.01 Hz and 1 Hz with a duty cycle between 0.01% and 10% duty cycle.

In some embodiments, the step of thermally actuating and the step of detecting temperature are carried out sequentially, wherein each of the one or more thermal elements actuates then detects.

In some embodiments, the step of thermally actuating is carried out by a first portion of the one or more thermal elements and the step of detecting temperature is carried out by a second portion of the one or more thermal elements. In some embodiments, the steps of thermally actuating and detecting temperature occur sequentially. In some embodiments, the step of detecting one or more temperatures comprises simultaneously obtaining signals from at least a portion of the second portion of the one or more thermal elements.

In some embodiments, the step of detecting one or more temperatures occurs at a frequency selected from the range of 0.0001 s⁻¹ to 1000 s⁻¹, or 0.001 s⁻¹ to 100 s⁻¹, or 0.01 s⁻¹ to 10 s⁻¹.

In some embodiments, the step of detecting one or more temperatures provides a temperature measurement characterized by a temporal resolution selected from 1 ms to 1000 s, or 10 ms to 100 s, or 100 ms to 10 s. In some embodiments, the step of detecting one or more temperatures provides a temperature measurement characterized by a spatial resolution selected from 0.01 mm to 1 cm, or from 0.1 mm to 0.1 cm. In some embodiments, the step of detecting one or more temperatures provides a temperature measurement characterized by a thermal resolution selected from 0.001° C. to 10° C. or 0.01° C. to 1° C. In some embodiments, the step of thermally actuating may increase the temperature of epidermal tissue 6° C. to 8° C. In practice, the amount of thermal actuation is controlled to prevent burning or skin discomfort while applying a signal strong enough to overcome background noise.

In some embodiments, the step of thermally actuating increases the temperatures of the epidermal tissue by less than 20° C., or less than 10° C., or less than 5° C., or less than 1° C.

In some embodiments, the step of detecting one or more temperatures corresponds to tissue having temperatures selected from the range of 0° C. to 50° C., or 10° C. to 40° C., or 20° C. to 38° C.

In some embodiments, methods disclosed herein further comprise a step of determining one or more thermal transport properties of the epidermal tissue using one or more temperatures of the epidermal tissue. For example, the thermal transport property may be thermal conductivity, thermal diffusivity or heat capacity per unit volume. In an embodiment, the one or more thermal transport properties are determined using one or more of the relationships:

$$T = {T}_{∞} + {A}_{1} \frac{Q}{2 π {A}_{2} {k}_{skin}} erfc ( \frac{{A}_{2} \sqrt{{ρ}_{skin} {c}_{p , skin}}}{\sqrt{4 {k}_{skin} t}} ) \qquad ( 1 )$$

where T_{∞} is the temperature before heating, Q is the heating power, kₛₖᵢₙ is the thermal conductivity of the skin, ρₛₖᵢₙc_{p,skin} is the volumetric heat capacity of skin, t is time, erfc is the complementary error function, A₂ represents the effective distance from the thermal actuator, and A₁ is a parameter that accounts for details associated with the multilayered geometry of the device;

$$T = {T}_{∞} + {A}_{1} \frac{{\int }_{{r}_{1}}^{{r}_{2}} \{ \frac{Q}{2 π {rk}_{skin}} erfc ( \frac{r \sqrt{{ρ}_{skin} {c}_{p , skin}}}{\sqrt{4 {k}_{skin} t}} ) dr \}}{{r}_{2} - {r}_{1}} \qquad ( 2 )$$

where T is the temperature at a sensor some distance away from the actuator, T_{∞} is the temperature before heating, Q is the heating power, kₛₖᵢₙ is the thermal conductivity of the skin, ρₛₖᵢₙc_{p,skin} is the volumetric heat capacity of skin, t is time, erfc is the complementary error function, r₁ is distance between the actuator and near edge of the sensor to the actuator, r₂ is distance between the actuator and near edge of the sensor to the actuator, and A₁ is a parameter that accounts for details associated with the multilayered geometry of the device; and

$$T = {T}_{∞} + {A}_{1} \frac{Q}{2 π r ( t ) {k}_{skin}} erfc ( \frac{r ( t ) \sqrt{{ρ}_{skin} {c}_{p , skin}}}{\sqrt{4 {k}_{skin} t}} ) \qquad ( 3 )$$

where T_{∞} is the temperature before heating, Q is the heating power, kₛₖᵢₙ is the thermal conductivity of the skin, ρₛₖᵢₙc_{p,skin} is the volumetric heat capacity of skin, t is time, erfc is the complementary error function, A₁ is a parameter that accounts for details associated with the multilayered geometry of the device, and r(t) represents the effective distance of the thermal sensor from the thermal actuator.

In some embodiments, methods disclosed herein further comprise determining one or more tissue parameters using the thermal transport property. For example, the one or more tissue parameters may be a physiological tissue parameter or a physical property of the tissue, such as a tissue parameter selected from the group consisting of hydration state, stratum corneum thickness, epidermis thickness and vasculature structure. In some embodiments, the hydration state has independent linear relationships with thermal conductivity and thermal diffusivity.

In some embodiments, methods disclosed herein further comprise determining the health of the epidermal tissue or determining the presence, absence or stage of a disease condition for the epidermal tissue of the subject. For example, the disease condition may be melanoma, rosacea or hyperpigmentation.

In some embodiments, methods disclosed herein further comprise steps of applying a dermatological compound to the surface of the epidermal tissue of the subject and analyzing the tissue temperatures to determine a clinical effectiveness or safety of a dermatological compounds on the tissue. For example, the epidermal tissue may be a follicular tissue or a palmar tissue that corresponds to the face, torso, arms, legs, back, hands or foot of the subject.

In some embodiments, methods disclosed herein further comprise contacting a device comprising the one or more thermal elements with a receiving surface of the epidermal tissue, wherein contact results in conformal contact with the receiving surface, thereby providing the one or more thermal elements in thermal contact with the epidermal tissue. In some embodiments, the step of contacting provides a contact area of the device with the epidermal tissue surface having an area selected from the range of 0.0001 mm² to 1 cm², or 0.001 mm² to 0.1 cm², or 0.01 mm² to 0.01 cm².

In an aspect, a wearable device comprises a flexible substrate including a multiplexed sensor array, the multiplexed sensor array having first circuitry configured to detect changes in temperature in response to thermal actuation and second circuitry configured to determine one or more tissue thermal properties responsive to detected temperatures, the tissue thermal properties including at least three-dimensional tissue thermal information.

In an embodiment, the first circuitry is configured to detect shifts in turn-ON voltage or electrical resistivity responsive to the changes in temperature.

In an embodiment, the second circuitry is configured to determine the one or more tissue thermal properties responsive to the detected shifts in turn-ON voltage or electrical resistivity. In some embodiments, the second circuitry configured to determine one or more tissue thermal properties responsive to the detected shifts in turn-ON voltage or electrical resistivity comprises one or more transducers. In an embodiment, the second circuitry configured to determine one or more tissue thermal properties responsive to the detected shifts in turn-ON voltage comprises one or more acoustic transducers, electroacoustic transducers, electrochemical transducers, electromagnetic transducers, electromechanical transducers, electrostatic transducers, photoelectric transducers, radioacoustic transducers, thermoelectric transducers, or ultrasonic transducers.

In some embodiments, the multiplexed sensor array includes a plurality of transducers interconnected so as to enable multiplexed addressing. In some embodiments, the multiplexed sensor array includes a plurality of sensors interconnected so as to enable multiplexed addressing.

In an embodiment, the one or more tissue thermal properties include one or more of a thermal conductivity, a thermal diffusivity, a tissue temperature, a regional temperature, temperature spatial distribution information, or temperature temporal information. In some embodiments, the one or more tissue thermal properties include tissue thermograph information.

In an embodiment, a wearable device further comprises circuitry configured to generate a thermal interrogating stimulus. In an embodiment, the circuitry configured to generate the thermal interrogating stimulus includes one or more thermal actuators.

In an embodiment, a wearable device further comprises circuitry configured to determine one or more tissue thermal properties responsive to the thermal interrogating stimulus.

In an embodiment, a wearable device further comprises an encapsulant that mimics one or more physical properties of skin. For example, the encapsulant may include at least one of a color, density, or texture that reduces the ability of a person to discriminate between the wearable device and skin.

In an embodiment, a wearable device further comprises circuitry configured to determine tissue dielectric information responsive to an applied voltage. For example, the circuitry configured to determine tissue dielectric information responsive to an applied voltage may include circuitry configured to determine tissue conductivity information or tissue permittivity information responsive to an applied voltage. In an embodiment, the circuitry configured to determine tissue dielectric information responsive to an applied voltage includes circuitry configured to determine tissue hydration information responsive to an applied voltage.

In an embodiment, a wearable device further comprises circuitry configured to activate a discovery protocol that allows a client device and the wearable device to identify each other and to negotiate information.

In an embodiment, a wearable device further comprises circuitry configured to activate a discovery protocol that allows an enterprise server and the wearable device to identify each other and to exchange information.

In an embodiment, circuitry includes, among other things, one or more computing devices such as a processor (e.g., a microprocessor, a quantum processor, qubit processor, etc.), a central processing unit (CPU), a digital signal processor (DSP), an application-specific integrated circuit (ASIC), a field programmable gate array (FPGA), or the like, or any combinations thereof, and can include discrete digital or analog circuit elements or electronics, or combinations thereof. In an embodiment, a module includes one or more ASICs having a plurality of predefined logic components. In an embodiment, a module includes one or more FPGAs, each having a plurality of programmable logic components.

In an embodiment, circuitry includes one or more components operably coupled (e.g., communicatively, electromagnetically, magnetically, ultrasonically, optically, inductively, electrically, capacitively coupled, wirelessly coupled, or the like) to each other. In an embodiment, circuitry includes one or more remotely located components. In an embodiment, remotely located components are operably coupled, for example, via wireless communication. In an embodiment, remotely located components are operably coupled, for example, via one or more communication modules, receivers, transmitters, transceivers, or the like.

In an embodiment, circuitry includes memory that, for example, stores instructions or information. Non-limiting examples of memory include volatile memory (e.g., Random Access Memory (RAM), Dynamic Random Access Memory (DRAM), or the like), non-volatile memory (e.g., Read-Only Memory (ROM), Electrically Erasable Programmable Read-Only Memory (EEPROM), Compact Disc Read-Only Memory (CD-ROM), or the like), persistent memory, or the like. Further non-limiting examples of memory include Erasable Programmable Read-Only Memory (EPROM), flash memory, or the like. In an embodiment, memory is coupled to, for example, one or more computing devices by one or more instructions, information, or power buses.

In an embodiment, circuitry includes one or more computer-readable media drives, interface sockets, Universal Serial Bus (USB) ports, memory card slots, or the like, and one or more input/output components such as, for example, a graphical user interface, a display, a keyboard, a keypad, a trackball, a joystick, a touch-screen, a mouse, a switch, a dial, or the like, and any other peripheral device. In an embodiment, a module includes one or more user input/output components that are operably coupled to at least one computing device configured to control (electrical, electromechanical, software-implemented, firmware-implemented, or other control, or combinations thereof) at least one parameter associated with, for example, determining one or more tissue thermal properties responsive to detected shifts in turn-ON voltage.

In an embodiment, circuitry includes a computer-readable media drive or memory slot that is configured to accept signal-bearing medium (e.g., computer-readable memory media, computer-readable recording media, or the like). In an embodiment, a program for causing a system to execute any of the disclosed methods can be stored on, for example, a computer-readable recording medium, a signal-bearing medium, or the like. Non-limiting examples of signal-bearing media include a recordable type medium such as a magnetic tape, floppy disk, a hard disk drive, a Compact Disc (CD), a Digital Video Disk (DVD), Blu-Ray Disc, a digital tape, a computer memory, or the like, as well as transmission type medium such as a digital or an analog communication medium (e.g., a fiber optic cable, a waveguide, a wired communications link, a wireless communication link (e.g., receiver, transmitter, transceiver, transmission logic, reception logic, etc.). Further non-limiting examples of signal-bearing media include, but are not limited to, DVD-ROM, DVD-RAM, DVD+RW, DVD-RW, DVD-R, DVD+R, CD-ROM, Super Audio CD, CD-R, CD+R, CD+RW, CD-RW, Video Compact Discs, Super Video Discs, flash memory, magnetic tape, magneto-optic disk, MINIDISC, non-volatile memory card, EEPROM, optical disk, optical storage, RAM, ROM, system memory, web server, or the like.

In an embodiment, circuitry includes acoustic transducers, electroacoustic transducers, electrochemical transducers, electromagnetic transducers, electromechanical transducers, electrostatic transducers, photoelectric transducers, radioacoustic transducers, thermoelectric transducers, or ultrasonic transducers.

In an embodiment, circuitry includes electrical circuitry operably coupled with a transducer (e.g., an actuator, a motor, a piezoelectric crystal, a Micro Electro Mechanical System (MEMS), etc.) In an embodiment, circuitry includes electrical circuitry having at least one discrete electrical circuit, electrical circuitry having at least one integrated circuit, or electrical circuitry having at least one application specific integrated circuit. In an embodiment, circuitry includes electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of memory (e.g., random access, flash, read only, etc.)), electrical circuitry forming a communications device (e.g., a modem, communications switch, optical-electrical equipment, etc.), and/or any non-electrical analog thereto, such as optical or other analogs.

In an aspect, a device for sensing epidermal tissue of a subject comprises: a stretchable or flexible substrate; one or more thermal elements supported by the flexible or stretchable substrate, the one or more thermal elements for: thermally actuating the tissue with the one or more thermal elements by delivering a heating power selected from the range of 0.0001 mJ s⁻¹ and 1000 mJ s⁻¹ for a period selected from the range of 10 ms to 1000 s; detecting one or more temperatures of the epidermal tissue proximate to the tissue region with the one or more thermal elements; and generating a depth profile thermal measurement; wherein the flexible or stretchable substrate and the one or more thermal elements provide a net bending stiffness low enough such that the device is capable of establishing conformal contact with a receiving surface of the epidermal tissue.

In some embodiments, a device for sensing epidermal tissue further comprises a processor in communication with one or more of the thermal elements for receiving and analyzing the temperature measurements to determine one or more thermal transport properties or tissue properties.

In some embodiments, the thermal elements of the device are at least partially encapsulated in the substrate or one or more encapsulation layers. In some embodiments, the thermal elements comprise stretchable or flexible structures. In some embodiments, the thermal elements comprise thin film structures. In some embodiments, the thermal elements comprise filamentary metal structures.

In some embodiments, the device has a modulus within a factor of 1000 of a modulus of the epidermal tissue at the interface with the device, or within a factor of 100 of a modulus of the epidermal tissue, or within a factor of 10 of a modulus of the epidermal tissue. In some embodiments, the device has an average modulus less than or equal to 100 MPa, or less than or equal to 10 MPa. In some embodiments, the device has an average thickness less than or equal to 3000 microns, or less than or equal to 300 microns, or less than or equal to 100 microns. In some embodiments, the device has a net bending stiffness less than or equal to 1 mN m, or less than or equal to 0.5 mN m. In some embodiments, the device exhibits a stretchability without failure of greater than 5%, or greater than 10%, or greater than 15%.

In some embodiments, device disclosed herein further comprise a first electrode for electrically actuating a first epidermal tissue region and a second electrode for obtaining an electrical signal from a second epidermal tissue region. In some embodiments, the first and second electrodes are in direct contact with the epidermal tissue. In some embodiments, the first electrode and the second electrode are separated by a distance selected from the range of 50 μm to 10 mm.

In some embodiments, the device further comprises one or more amplifiers, strain gauges, temperature sensors, wireless power coils, solar cells, inductive coils, high frequency inductors, high frequency capacitors, high frequency oscillators, high frequency antennae, multiplex circuits, electrocardiography sensors, electromyography sensors, electroencephalography sensors, electrophysiological sensors, thermistors, transistors, diodes, resistors, capacitive sensors, light emitting diodes, superstrate, embedding layers, encapsulating layers, planarizing layers or any combinations of these.

In some embodiments, thermal sensing configurations comprise planar thermal sensing/actuating elements electronically and/or thermally connected by individual wire segments having widths ranging from 20 μm to 50 μm and lengths ranging from 1 mm to 10 mm. In typical embodiments, sensor element spacings range from 50 μm to 1 cm. Actuators may have the same geometry as the sensors so long as the relationship

$$Q = {I}^{2} ρ \frac{L}{A}$$

is obeyed, where Q is the actuating power, ρ is the resitivity (material property of the actuating element), L is the length of the actuating wire and A is the cross sectional area of the actuating wire. The length of the actuator wire can assume a wide range of values to provide suitable actuating powers for a given input current. Alternatively, any given sensor can be used as an actuator by increasing the actuating current provided to that sensor, which follows a strong quadratic relationship with actuating power.

In some embodiments, impedance/electrical sensing and actuating configurations comprise a radial inner electrode and an annular outer electrode. For example, the radius of the inner electrode may range from 25 μm to 200 μm and the radius of outer electrode may range from 100 μm to 1 mm. Typical electrode spacings are from 1 mm to 5 mm. In some embodiments, an electrical sensing/actuating device can be configured to have a reference electrode in contact with a material with known dielectric properties, and the remaining electrodes may be in contact with the skin to provide a differential impedance measurement.

In an aspect, the present invention is a method for determining a thermal property of a epidermal tissue, the method comprising: thermally actuating the epidermal tissue with one or more thermal actuators of a device in conformal contact with the epidermal tissue; measuring temperature of the epidermal tissue with one or more thermal sensors of the device; determining an effective distance of the one or more thermal sensors from the one or more thermal actuators; and utilizing the effective distance to determine the thermal transport property of the epidermal tissue.

In an aspect, the present invention is a method for analyzing clinical effectiveness or safety of dermatological compounds on epidermal tissue, the method comprising: (i) thermally actuating the epidermal tissue with one or more thermal actuators of a device in conformal contact with the epidermal tissue; (ii) measuring temperature of the epidermal tissue with one or more thermal sensors of the device; (iii) determining an effective distance of the one or more thermal sensors from the one or more thermal actuators; (iv) utilizing the effective distance to determine a thermal transport property of the epidermal tissue; (v) applying a dermatological compound to the epidermal tissue; and (vi) repeating steps (i)-(v).

In some embodiments, the effective distance of the one or more thermal sensors from the one or more thermal actuators is a time-dependent value.

In some embodiments, the step of determining an effective distance of the one or more thermal sensors from the one or more thermal actuators comprises subtracting a response of the thermal sensor furthest from the thermal actuator from that of each of the thermal sensors in the device to minimize effects of fluctuations in ambient temperature.

In some embodiments, methods disclosed herein further comprise using Eq. (1) to determine a thermal transport property of the tissue

$$T = {T}_{∞} + {A}_{1} \frac{Q}{2 π {A}_{2} {k}_{skin}} erfc ( \frac{{A}_{2} \sqrt{{ρ}_{skin} {c}_{p , skin}}}{\sqrt{4 {k}_{skin} t}} ) \qquad ( 1 )$$

where T_{∞} is the temperature before heating, Q is the heating power, kₛₖᵢₙ is the thermal conductivity of the skin, ρₛₖᵢₙc_{p,skin} is the volumetric heat capacity of skin, t is time, erfc is the complementary error function, A₂ represents the effective distance from the thermal actuator, and A₁ is a parameter that accounts for details associated with the multilayered geometry of the device.

In some embodiments, methods disclosed herein further comprise using Eq. (3) to determine a thermal transport property of the tissue

$$T = {T}_{∞} + {A}_{1} \frac{Q}{2 π r ( t ) {k}_{skin}} erfc ( \frac{r ( t ) \sqrt{{ρ}_{skin} {c}_{p , skin}}}{\sqrt{4 {k}_{skin} t}} ) \qquad ( 3 )$$

where T_{∞} is the temperature before heating, Q is the heating power, kₛₖᵢₙ is the thermal conductivity of the skin, ρₛₖᵢₙc_{p,skin} is the volumetric heat capacity of skin, t is time, erfc is the complementary error function, A₁ is a parameter that accounts for details associated with the multilayered geometry of the device, and r(t) represents the effective distance of the thermal sensor from the thermal actuator.

In an aspect, the present invention is a method of sensing an epidermal tissue of a subject, the method comprising: thermally actuating the epidermal tissue with one or more elements of a device in conformal contact with the epidermal tissue; measuring temperature of the epidermal tissue with the one or more elements; electrically actuating the epidermal tissue with a first electrode of the device; and measuring voltage at a second electrode of the device.

In an embodiment, devices and methods disclosed herein may include in vivo administration of a device to epidermal tissue of a subject, such as a human or non-human subject. Administration may include direct administration where a device is provided in direct physical contact with epidermal tissue or administration may include using one or more intermediate materials or structures provided between the device and the epidermal tissue, such as using adhesives and other bonding or interfacing media. In an embodiment, a method of may include a step of administration a device to the external surface of epidermal tissue of a subject, for example, the torso, face, neck, feet, legs and other body locations.

In some embodiments, the devices may be administered to a subject in need of diagnostic or therapeutic treatment or monitoring. Examples of diagnostic procedures include, for example, identification of the onset or stage of a disease condition or the characterization of susceptibility to disease conditions.

### BRIEF DESCRIPTION OF THE DRAWINGS

FIG. 1: Ultrathin, conformal device for evaluating thermal transport characteristics and validation on human skin. (a) Photograph of a device laminated onto a subject's cheek. (b) Magnified view showing the location of the heater, a sensing element 3.5 mm away, 4.7 mm away, and 5.8 mm away from the heater. (c) Magnified view during deformation. (d) Optical coherence tomography image of a region of a human palm before and (e) after mounting the array.

FIG. 2: Thermal flow associated with low level transient heating on the surface of the skin is an example of three-dimensional tissue thermal information. (a) Infrared image during heating at a single thermal actuator in an array device on the skin. (b) Finite element modelling results for the distribution of temperature during rapid, low level heating at an isolated actuator on the skin, after 1.2 s of heating at a power of 3.7 mW mm⁻². (c) Spatial map of a depth profile thermal measurement showing the rise in temperature due to transient heating sequentially in each element in the array. The solid black lines are experimental data; the red dashed lines are best fit calculations. The strong rise shown in upper leftmost element results from local delamination of the device from the skin. (d) Experimental data (solid lines) and best fit calculations (dashed lines) for the cheek and heel, along with extracted thermal transport properties.

FIG. 3: Clinical data distributions. Boxplot representation of the data (open circles). The mean is represented by a black diamond shape. The top and the bottom line of the box are the first and third quartiles, and the middle line of the box is the second quartile—the median. The lower (upper) whisker represents the minimum (maximum) observation above (below) the 1.5 Inter Quartile Range (IQR) below (above) the lower (upper) quartile. Data distributions are shown for the (a) stratum corneum thickness (SC-thick), (b) stratum corneum hydration (SC-h), (c) epidermis thickness (EP-thick), (d) thermal conductivity (k), (e) volumetric heat capacity (ρcₚ), and (f) thermal diffusivity (α).

FIG. 4: Clinical data correlation analysis. (a) Scatterplot matrix representation for the entire data set (all 6 body locations: cheek, volar and dorsal forearm, wrist, palm, and heel on 25 total subjects). Pairwise correlation analyses include the thermal characteristics (k, W m⁻¹° C.⁻¹; ρcₚ, J cm⁻³° C.⁻¹; α, mm² s⁻¹) and stratum corneum thickness (SC-thick, μm), epidermal thickness (EP-thick, μm), and stratum corneum hydration (SCh, arbitrary units). Data for different body areas are represented by different colors. The red line represents the pairwise linear regression slope. The pink shaded clouds represent the 95% bivariate normal density ellipse. Assuming the variables are bivariate normally distributed, this ellipse encloses approximately 95% of the points. (b) The bivariate correlations for the entire data set are represented using a color coding (HeatMap) scheme associated with a clustering of the descriptors. Dark red is associated with Pearson Correlation Coefficient, R, equal to 1 and dark blue is associated to R=−1. The Pearson correlation coefficients are given in Table 2.

FIG. 5: Clinical data correlation analysis for regions without significant stratum corneum thickness. The same correlation analysis as in FIG. 4 for the (a) cheek, (b) dorsal forearm, (c) volar forearm and (d) wrist.

FIG. 6: Clinical data correlation analysis for regions with significant stratum corneum thickness. The same correlation analysis as in FIG. 4 for the (a) palm and (b) heel.

FIG. 7: Principal Component Analysis. Global, multivariate correlation analysis. On the biplot each body location is represented by polygons and the descriptors by triangles.

FIG. 8: Spatial mapping of thermal transport associated with low level heating on the surface of the skin. (a) Spatial map of the changes in temperature at each sensor (i.e. element) in the array. The data processing uses an adjacent-average filter (window size=8 s) and normalization to Element 16. The red highlight and colored boxes represent the elements boxed in the same colors in FIG. 1b. (b) Change in temperature at elements 3.5 mm away (blue), 4.7 mm away (black) and 5.8 mm away (red) from the element responsible for thermal actuation. The solid and dashed lines represent experimental data and best fit calculations, with k˜0.35-0.43 W m⁻¹ K⁻¹ and α˜0.12-0.15 mm² s⁻¹. (c) Results of finite element modelling of an array on a cheek, in the same arrangement as b.

FIG. 9: Anisotropic convective effects associated with near surface blood flow. (a) Spatial map of changes in temperature at each element for a device located at the volar aspect of the wrist. The position of the thermal actuator coincides with a large vein. (b) Difference in temperature between element 11 (E11) and element 3 (E3). The results show effects of anisotropic heat flow in the wrist, compared to isotropic distributions typically observed on a region of the body such as the cheek. The vertical dashed lines correspond to initiation and termination of heating, respectively.

FIG. 10: Device construction and temperature comparison to IR measurements. (a) Optical image of 4×4 thermal sensing array, showing the bonding location of the thin, flexible cable (ACF connection). (b) Magnified image of a single sensor/actuator element, showing the 10 μm wide, serpentine configuration. (c) Cross sectional schematic showing the device layout on skin. (d) Comparison of temperature device readings on six body locations on each of twenty-five subjects, as compared to IR measurements. Pearson correlation coefficient=0.98.

FIG. 11: Representative photographs of each body location before, during, and after measurements. Images show each body location before application of the thermal sensing array, with the device applied to skin during heating applications for thermal measurements, and then after device removal. No irritation is observed as a result of heating, or wearing the device. Body locations are (a) cheek, (b) volar forearm, (c) dorsal forearm, (d) wrist, (e) palm, and (f) heel.

FIG. 12: Temperature variations across body locations. (a) Variation in temperature data between different subjects on different body locations for thermal sensing array (left) and IR thermometer (right). (b) Inter- and intra-subject variance for the thermal sensing array and IR thermometer.

FIGS. 13A-13F: Temperature variations across body locations for each subject. Variation in temperature data between different subjects on different body locations for thermal sensing array (blue) and IR thermometer (red).

FIG. 14: Analysis of fitting process sensitivity with experimental error. (a) Experimental precision fitting error analysis of representative in vivo data on a human heel. Experimental error range is given by 3× the standard deviation of temperature readings from the mean. (b) Experimental accuracy fitting error analysis of representative in vivo data on a human heel and (c) a human cheek. Experimental error range is given by the 95% confidence interval of temperature readings due to calibration errors.

FIG. 15: Experimental determination of measurement probing depth. Measured thermal conductivities by the thermal sensing array for different thickness of a silicone with thermal properties similar to skin (Sylgard 170, Dow Corning, USA; k=0.39 W m⁻¹ K⁻¹, ρ=1370 kg m⁻³) on copper. The measured thermal conductivity rises rapidly when the silicone layer becomes thinner than the probing depth, which is given by Eq. 2 to be approximately 0.5 mm.

FIG. 16: Solutions for r(t). Numerically determined solutions for r(t) over the appropriate measurement time, determined using k=0.35 W m⁻¹ K⁻¹ and α=0.15 mm² s⁻¹, for (a) r=˜3.5 mm, (b) r=˜4.7 mm, and (c) r=˜5.8 mm. (d) Example temperature rise solutions for a sensor ˜3.5 mm away using the integrated solution of Eq. S5, r(t) given in Eq. S6, and various time independent values of r with Eq. S6. r(t) gives the smallest discrepancy with Eq. S5 at <1%, and time independent average values of r give discrepancies <5%.

FIGS. 17A-17C: Principle component analysis. Boxplot representation of principal components by body location, and their corresponding relation to measured parameters. FIG. 17A, Box plots and correlation weights of the first principal component; FIG. 17B, the second principal component; and FIG. 17C, the third principal component.

FIG. 18: Corneometer (CM 825®, Courage+Khazaka electronic GmbH) measurement (capacitance-based measurement) at locations where stimulus is applied at defined time points. Shows strong peak at TI time point for both age groups, probably corresponding to initial water evaporation from glycerine solution. Measurements reach baseline at Tend time point. Occlusive patch has much smaller effect, as expected. Measurement serves as main validation of experimental epidermal sensor being tested.

FIG. 19: Transepidermal Water Loss (TEWL) (Vapometer®, Delfin Technologies) measurements, for both age groups using defined time points and stimuli, as measured from stratum corneum. Data show a strong peak at TI, immediately after stimulus is applied, corresponding to loss in water in solution, consistent for both age groups. Occlusive patch has much smaller effect for both age groups, as expected.

FIG. 20: Skin thermal conductivity (kₛₖᵢₙ) measurements using an epidermal electronic system for both age groups using defined time points and stimuli. Shows a clear increase in thermal conductivity with hydration, as expected.

FIG. 21: Thermal diffusivity

$$( {α}_{skin} = \frac{{k}_{skin}}{{ρ}_{skin} {c}_{p , skin}} )$$

measurements using an epidermal electronic system for both age groups using defined time points and stimuli. Shows a decrease with increased hydration, due to increased specific heat capacity of skin with hydration.

FIG. 22: Impedance magnitude measurements

$$( {z}_{skin} = \frac{V}{I} )$$

using an epidermal electronic system for both age groups using defined time points and stimuli. Shows a strong decrease with increased hydration, as expected, suggesting peak hydration levels at either the T30 or T60 time points for both age groups.

FIG. 23: Impedance phase angle

$$( θ = {tan}^{- 1} ( \frac{V}{I} ) ) )$$

using an epidermal electronic system for both age groups using defined time points and stimuli. Can also be used as an indicator of hydration level.

FIG. 24: FIG. 18 replotted with TI (initial time point after stimulus is applied) as the baseline. Shows change in measured value after initial application of stimulus.

FIG. 25: FIG. 19 replotted with TI (initial time point after stimulus is applied) as the baseline. Shows change in measured value after initial application of stimulus.

FIG. 26: FIG. 20 replotted with TI (initial time point after stimulus is applied) as the baseline. Shows change in measured value after initial application of stimulus.

FIG. 27: FIG. 21 replotted with TI (initial time point after stimulus is applied) as the baseline. Shows change in measured value after initial application of stimulus.

FIG. 28: FIG. 22 replotted with TI (initial time point after stimulus is applied) as the baseline. Shows change in measured value after initial application of stimulus.

FIG. 29: FIG. 23 replotted with TI (initial time point after stimulus is applied) as the baseline. Shows change in measured value after initial application of stimulus.

FIGS. 30-34: Raw data for every patient for stimuli and measurement modes shown in FIGS. 18-29.

FIG. 35: Resistivity and dielectric constant as a function of measurement frequency for different layers of the skin. The subscript k refers to the stratum corneum and c refers to the underlying layers of viable skin. (Yamamoto, T. and Y. Yamamoto (1976). “Electrical properties of the epidermal stratum corneum.” Medical and Biological Engineering 14(2): 151-158.)

FIG. 36: Comparison of thermal conductivity and impedance data with commercial tool (Corneometer, CM-825, Courage+Khazaka gmbh), on 21 female subjects, across two age groups, 18-30 and 50-65.

### DETAILED DESCRIPTION OF THE INVENTION

In general, the terms and phrases used herein have their art-recognized meaning, which can be found by reference to standard texts, journal references and contexts known to those skilled in the art. The following definitions are provided to clarify their specific use in the context of the invention.

“Functional substrate” refers to a substrate component for a device having at least one function or purpose other than providing mechanical support for a component(s) disposed on or within the substrate. In an embodiment, a functional substrate has at least one skin-related function or purpose. In an embodiment, a functional substrate has a mechanical functionality, for example, providing physical and mechanical properties for establishing conformal contact at the interface with a tissue, such as skin. In an embodiment, a functional substrate has a thermal functionality, for example, providing a thermal loading or mass small enough so as to avoid interference with measurement and/or characterization of a physiological parameter. In an embodiment, a functional substrate of the present devices and method is biocompatible and/or bioinert. In an embodiment, a functional substrate may facilitate mechanical, thermal, chemical and/or electrical matching of the functional substrate and the skin of a subject such that the mechanical, thermal, chemical and/or electrical properties of the functional substrate and the skin are within 20%, or 15%, or 10%, or 5% of one another.

In some embodiments, a functional substrate that is mechanically matched to a tissue, such as skin, provides a conformable interface, for example, useful for establishing conformal contact with the surface of the tissue. Devices and methods of certain embodiments incorporate mechanically functional substrates comprising soft materials, for example exhibiting flexibility and/or stretchability, such as polymeric and/or elastomeric materials. In an embodiment, a mechanically matched substrate has a modulus less than or equal to 100 MPa, and optionally for some embodiments less than or equal to 10 MPa, and optionally for some embodiments, less than or equal to 1 MPa. In an embodiment, a mechanically matched substrate has a thickness less than or equal to 0.5 mm, and optionally for some embodiments, less than or equal to 1 cm, and optionally for some embodiments, less than or equal to 3 mm. In an embodiment, a mechanically matched substrate has a bending stiffness less than or equal to 1 nN m, optionally less than or equal to 0.5 nN m.

In some embodiments, a mechanically matched functional substrate is characterized by one or more mechanical properties and/or physical properties that are within a specified factor of the same parameter for an epidermal layer of the skin, such as a factor of 10 or a factor of 2. In an embodiment, for example, a functional substrate has a Young's Modulus or thickness that is within a factor of 20, or optionally for some applications within a factor of 10, or optionally for some applications within a factor of 2, of a tissue, such as an epidermal layer of the skin, at the interface with a device of the present invention. In an embodiment, a mechanically matched functional substrate may have a mass or modulus that is equal to or lower than that of skin.

In some embodiments, a functional substrate that is thermally matched to skin has a thermal mass small enough that deployment of the device does not result in a thermal load on the tissue, such as skin, or small enough so as not to impact measurement and/or characterization of a physiological parameter. In some embodiments, for example, a functional substrate that is thermally matched to skin has a thermal mass low enough such that deployment on skin results in an increase in temperature of less than or equal to 2 degrees Celsius, and optionally for some applications less than or equal to 1 degree Celsius, and optionally for some applications less than or equal to 0.5 degree Celsius, and optionally for some applications less than or equal to 0.1 degree Celsius. In some embodiments, for example, a functional substrate that is thermally matched to skin has a thermal mass low enough that is does not significantly disrupt water loss from the skin, such as avoiding a change in water loss by a factor of 1.2 or greater. Therefore, the device does not substantially induce sweating or significantly disrupt transdermal water loss from the skin.

In an embodiment, the functional substrate may be at least partially hydrophilic and/or at least partially hydrophobic.

In an embodiment, the functional substrate may have a modulus less than or equal to 100 MPa, or less than or equal to 50 MPa, or less than or equal to 10 MPa, or less than or equal to 100 kPa, or less than or equal to 80 kPa, or less than or equal to 50 kPa. Further, in some embodiments, the device may have a thickness less than or equal to 5 mm, or less than or equal to 2 mm, or less than or equal to 100 μm, or less than or equal to 50 μm, and a net bending stiffness less than or equal to 1 nN m, or less than or equal to 0.5 nN m, or less than or equal to 0.2 nN m. For example, the device may have a net bending stiffness selected from a range of 0.1 to 1 nN m, or 0.2 to 0.8 nN m, or 0.3 to 0.7 nN m, or 0.4 to 0.6 nN m.

A “component” is used broadly to refer to an individual part of a device.

In an embodiment, “coincident” refers to the relative position of two or more objects, planes, surfaces, regions or signals occurring together in space and time, including physically and/or temporally overlapping objects, planes, surfaces, regions or signals.

In an embodiment, “proximate” refers to the relative position of two objects, planes, surfaces, regions or signals that are closer in relationship than any one of those objects is to a third object of the same type as the second object. Proximate relationships include, but are not limited to, physical, electrical, thermal and/or optical contact. In an embodiment, epidermal tissue proximate to a thermal element is directly adjacent to the thermal element and closer to that thermal element than any other thermal element in an array of thermal elements. In an embodiment, two objects proximate to one another may be separated by a distance less than or equal to 50 mm, or less than or equal to 25 mm, or less than or equal to 10 mm, or two objects proximate to one another may be separated by a distance selected from the range of 0 mm to 50 mm, or 0.1 mm to 25 mm, or 0.5 mm to 10 mm, or 1 mm to 5 mm.

“Sensing” refers to detecting the presence, absence, amount, magnitude or intensity of a physical and/or chemical property. Useful device components for sensing include, but are not limited to electrode elements, chemical or biological sensor elements, pH sensors, temperature sensors, strain sensors, mechanical sensors, position sensors, optical sensors and capacitive sensors.

“Actuating” refers to stimulating, controlling, or otherwise affecting a structure, material or device component. Useful device components for actuating include, but are not limited to, electrode elements, electromagnetic radiation emitting elements, light emitting diodes, lasers, magnetic elements, acoustic elements, piezoelectric elements, chemical elements, biological elements, and heating elements.

The terms “directly and indirectly” describe the actions or physical positions of one component relative to another component. For example, a component that “directly” acts upon or touches another component does so without intervention from an intermediary. Contrarily, a component that “indirectly” acts upon or touches another component does so through an intermediary (e.g., a third component).

In an embodiment, “epidermal tissue” refers to the outermost layers of the skin or the epidermis. The epidermis is stratified into the following non-limiting layers (beginning with the outermost layer): stratum corneum, stratum lucidum (on the palms and soles, i.e., the palmar regions), stratum granulosum, stratum spinosum, stratum germinativum (also called the statum basale). In an embodiment, epidermal tissue is human epidermal tissue.

“Encapsulate” refers to the orientation of one structure such that it is at least partially, and in some cases completely, surrounded by one or more other structures, such as a substrate, adhesive layer or encapsulating layer. “Partially encapsulated” refers to the orientation of one structure such that it is partially surrounded by one or more other structures, for example, wherein 30%, or optionally 50%, or optionally 90% of the external surface of the structure is surrounded by one or more structures. “Completely encapsulated” refers to the orientation of one structure such that it is completely surrounded by one or more other structures.

“Dielectric” refers to a non-conducting or insulating material.

“Polymer” refers to a macromolecule composed of repeating structural units connected by covalent chemical bonds or the polymerization product of one or more monomers, often characterized by a high molecular weight. The term polymer includes homopolymers, or polymers consisting essentially of a single repeating monomer subunit. The term polymer also includes copolymers, or polymers consisting essentially of two or more monomer subunits, such as random, block, alternating, segmented, grafted, tapered and other copolymers. Useful polymers include organic polymers or inorganic polymers that may be in amorphous, semi-amorphous, crystalline or partially crystalline states. Crosslinked polymers having linked monomer chains are particularly useful for some applications. Polymers useable in the methods, devices and components disclosed include, but are not limited to, plastics, elastomers, thermoplastic elastomers, elastoplastics, thermoplastics and acrylates. Exemplary polymers include, but are not limited to, acetal polymers, biodegradable polymers, cellulosic polymers, fluoropolymers, nylons, polyacrylonitrile polymers, polyamide-imide polymers, polyimides, polyarylates, polybenzimidazole, polybutylene, polycarbonate, polyesters, polyetherimide, polyethylene, polyethylene copolymers and modified polyethylenes, polyketones, poly(methyl methacrylate), polymethylpentene, polyphenylene oxides and polyphenylene sulfides, polyphthalamide, polypropylene, polyurethanes, styrenic resins, sulfone-based resins, vinyl-based resins, rubber (including natural rubber, styrene-butadiene, polybutadiene, neoprene, ethylene-propylene, butyl, nitrile, silicones), acrylic, nylon, polycarbonate, polyester, polyethylene, polypropylene, polystyrene, polyvinyl chloride, polyolefin or any combinations of these.

“Elastomer” refers to a polymeric material which can be stretched or deformed and returned to its original shape without substantial permanent deformation. Elastomers commonly undergo substantially elastic deformations. Useful elastomers include those comprising polymers, copolymers, composite materials or mixtures of polymers and copolymers. Elastomeric layer refers to a layer comprising at least one elastomer. Elastomeric layers may also include dopants and other non-elastomeric materials. Useful elastomers include, but are not limited to, thermoplastic elastomers, styrenic materials, olefinic materials, polyolefin, polyurethane thermoplastic elastomers, polyamides, synthetic rubbers, PDMS, polybutadiene, polyisobutylene, poly(styrene-butadiene-styrene), polyurethanes, polychloroprene and silicones. Exemplary elastomers include, but are not limited to silicon containing polymers such as polysiloxanes including poly(dimethyl siloxane) (i.e. PDMS and h-PDMS), poly(methyl siloxane), partially alkylated poly(methyl siloxane), poly(alkyl methyl siloxane) and poly(phenyl methyl siloxane), silicon modified elastomers, thermoplastic elastomers, styrenic materials, olefinic materials, polyolefin, polyurethane thermoplastic elastomers, polyamides, synthetic rubbers, polyisobutylene, poly(styrene-butadiene-styrene), polyurethanes, polychloroprene and silicones. In an embodiment, a polymer is an elastomer.

“Conformable” refers to a device, material or substrate which has a bending stiffness that is sufficiently low to allow the device, material or substrate to adopt any desired contour profile, for example a contour profile allowing for conformal contact with a surface having a pattern of relief features. In certain embodiments, a desired contour profile is that of skin.

“Conformal contact” refers to contact established between a device and a receiving surface. In one aspect, conformal contact involves a macroscopic adaptation of one or more surfaces (e.g., contact surfaces) of a device to the overall shape of a surface. In another aspect, conformal contact involves a microscopic adaptation of one or more surfaces (e.g., contact surfaces) of a device to a surface resulting in an intimate contact substantially free of voids. In an embodiment, conformal contact involves adaptation of a contact surface(s) of the device to a receiving surface(s) such that intimate contact is achieved, for example, wherein less than 20% of the surface area of a contact surface of the device does not physically contact the receiving surface, or optionally less than 10% of a contact surface of the device does not physically contact the receiving surface, or optionally less than 5% of a contact surface of the device does not physically contact the receiving surface. Devices of certain aspects are capable of establishing conformal contact with tissue surfaces characterized by a range of surface morphologies including planar, curved, contoured, macro-featured and micro-featured surfaces and any combination of these. Devices of certain aspects are capable of establishing conformal contact with tissue surfaces corresponding to tissue undergoing movement.

“Young's modulus” is a mechanical property of a material, device or layer which refers to the ratio of stress to strain for a given substance. Young's modulus may be provided by the expression:

$$E = \frac{( stress )}{( strain )} = ( \frac{{L}_{0}}{Δ L} ) ( \frac{F}{A} ) , \qquad ( I )$$

where E is Young's modulus, L₀ is the equilibrium length, ΔL is the length change under the applied stress, F is the force applied, and A is the area over which the force is applied. Young's modulus may also be expressed in terms of Lame constants via the equation:

$$E = \frac{μ ( 3 λ + 2 μ )}{λ + μ} , \qquad ( II )$$

where λ and μ are Lame constants. High Young's modulus (or “high modulus”) and low Young's modulus (or “low modulus”) are relative descriptors of the magnitude of Young's modulus in a given material, layer or device. In some embodiments, a high Young's modulus is larger than a low Young's modulus, preferably about 10 times larger for some applications, more preferably about 100 times larger for other applications, and even more preferably about 1000 times larger for yet other applications. In an embodiment, a low modulus layer has a Young's modulus less than 100 MPa, optionally less than 10 MPa, and optionally a Young's modulus selected from the range of 0.1 MPa to 50 MPa. In an embodiment, a high modulus layer has a Young's modulus greater than 100 MPa, optionally greater than 10 GPa, and optionally a Young's modulus selected from the range of 1 GPa to 100 GPa. In an embodiment, a device of the invention has one or more components having a low Young's modulus. In an embodiment, a device of the invention has an overall low Young's modulus.

“Low modulus” refers to materials having a Young's modulus less than or equal to 10 MPa, less than or equal to 5 MPa or less than or equal to 1 MPa.

“Bending stiffness” is a mechanical property of a material, device or layer describing the resistance of the material, device or layer to an applied bending moment. Generally, bending stiffness is defined as the product of the modulus and area moment of inertia of the material, device or layer. A material having an inhomogeneous bending stiffness may optionally be described in terms of a “bulk” or “average” bending stiffness for the entire layer of material.

In an embodiment, “tissue parameter” refers to a property of a tissue including a physical property, physiological property, electronic property, optical property and/or chemical composition. Non-limiting examples of tissue parameters include a surface property, a sub-surface property or a property of a material derived from the tissue, such as a biological fluid. For example, the term “tissue parameter” may refer to a parameter corresponding to an in vivo tissue such as temperature; hydration state; chemical composition of the tissue; intensity of electromagnetic radiation exposed to the tissue; and wavelength of electromagnetic radiation exposed to the tissue. Devices of some embodiments are capable of generating a response that corresponds to one or more tissue parameters.

In an embodiment, “environmental parameter” refers to a property of an environment of a device, such as a device in conformal contact with a tissue. Environment parameter may refer to a physical property, electronic property, optical property and/or chemical composition, such as an intensity of electromagnetic radiation exposed to the device; wavelengths of electromagnetic radiation exposed to the device; amount of humidity exposed to the device, ambient temperature exposed to the device. Devices of some embodiments are capable of generating a response that corresponds to one or more environmental parameters.

In an embodiment, “thermal transport property” refers to a rate of change of a temperature-related tissue property, such as a heat-related tissue property, over time and/or distance (velocity). In some embodiments, the heat-related tissue property may be temperature, conductivity or humidity. The heat-related tissue property may be used to determine a thermal transport property of the tissue, where the “thermal transport property” relates to heat flow or distribution at or near the tissue surface. In some embodiments, thermal transport properties include temperature distribution across a tissue surface, thermal conductivity, thermal diffusivity and heat capacity. Thermal transport properties, as evaluated in the present methods and systems, may be correlated with a physical or physiological property of the tissue. In some embodiments, a thermal transport property may correlate with a temperature of tissue. In some embodiments, a thermal transport property may correlate with a vasculature property, such as blood flow and/or direction.

In an embodiment, “effective distance” refers to an approximated physical distance between two points (e.g., objects or device components), such as a median or average distance between two points. In another embodiment, an effective distance between two points is a function of a second parameter, e.g., distance as a function of time, temperature, hydration, thermal properties and skin depth.

In an embodiment, “depth profile thermal measurement” refers to sensing, measurement or other characterization of one or more thermal transport properties of tissue, such as thermal conductivity, thermal diffusivity or heat capacity, as a function of depth within a tissue. In some embodiments, a depth profile thermal measurement includes measurement of one or more thermal transport properties for a layer of tissue having a certain thickness and located a certain distance from the tissue surface. In some embodiments, for example, a depth profile thermal measurement includes measurement of one or more thermal transport properties for at least two layers within a tissue corresponding to different depths relative to an external surface of the tissue. In some embodiments, for example, a depth profile thermal measurement includes measurement of one or more thermal transport properties corresponding to different penetration depths within a tissue relative to an external surface of the tissue. In some embodiments, for example, a depth profile thermal measurement includes measurements of one or more thermal transport properties corresponding to a three dimensional tissue location, for example, relative to the position of a tissue mounted device or device component thereof. Non-limiting depth profile thermal measurements of the invention may further include a spatial component corresponding to a lateral position on a tissue surface, for example, relative to the position of a tissue mounted device or device component thereof. In some embodiment, depth profile thermal measurements of the invention may further include a temporal component corresponding to one or more measurement times.

In an embodiment, a “depth profile” as used herein refers to characterization of epidermal tissue along an axis perpendicular to the epidermal tissue surface, i.e., throughout a thickness of the epidermal tissue.

In an embodiment, three-dimensional tissue thermal information refers to one or more thermal transport properties of tissue, such as thermal conductivity, thermal diffusivity or heat capacity, as a function of three dimensional tissue location, for example relative to the position of a tissue mounted device or device component thereof.

In an embodiment, three-dimensional hydration profile refers to measurements of tissue hydration state as a function of three dimensional tissue location, for example relative to the position of a tissue mounted device or device component thereof.

In an embodiment, three-dimensional circulation profile refers to measurements of tissue circulation property, such as blood flow rate or direction, as a function of three dimensional tissue location, for example relative to the position of a tissue mounted device or device component thereof.

The invention can be further understood by the following non-limiting examples.

### Example 1: Thermal Transport Characteristics of Human Skin Measured In Vivo Using Ultrathin Conformal Arrays of Thermal Sensors and Actuators

Measurements of the thermal transport properties of the skin can reveal changes in physical and chemical states of relevance to dermatological health, skin structure and activity, thermoregulation and other aspects of human physiology. Existing methods for in vivo evaluations demand complex systems for laser heating and infrared thermography, or they require rigid, invasive probes; neither can apply to arbitrary regions of the body, offers modes for rapid spatial mapping, or enables continuous monitoring outside of laboratory settings. Here we describe human clinical studies using mechanically soft arrays of thermal actuators and sensors that laminate onto the skin to provide rapid, quantitative in vivo determination of both the thermal conductivity and thermal diffusivity, in a completely non-invasive manner. Comprehensive analysis of measurements on six different body locations of each of twenty-five human subjects reveal systematic variations and directional anisotropies in the characteristics, with correlations to the thicknesses of the epidermis (EP) and stratum corneum (SC) determined by optical coherence tomography, and to the water content assessed by electrical impedance based measurements. Multivariate statistical analysis establishes four distinct locations across the body that exhibit different physical properties: heel, cheek, palm, and wrist/volar forearm/dorsal forearm. The data also demonstrate that thermal transport correlates negatively with SC and EP thickness and positively with water content, with a strength of correlation that varies from region to region, e.g. stronger in the palmar than in the follicular regions.

Skin is the largest organ of the human body and it provides one of the most diverse sets of functions. The outermost layer, the stratum corneum (SC), serves as a protective barrier and the first defense against physical, chemical and biological damage. The skin also receives and processes multiple sensory stimuli, such as touch, pain and temperature and aids in the control of body temperature and the flow of fluids in/out of the body′. These processes are highly regulated by nervous and circulatory systems, but also depend directly and indirectly on thermal characteristics. The thermal transport properties of this tissue system can reflect physical/chemical states of the skin, with potentially predictive value in contexts ranging from dermatology to cosmetology. Measurement systems for ex vivo analysis^{2,3} have some utility in establishing a general understanding of the properties, but they are irrelevant to investigations of the skin as an integral part of a complex, living organism. Existing in vivo approaches couple the use of laser heating or induced changes in the ambient temperature with infrared thermography⁴⁻⁶, or they exploit rigid probes that press against the skin^{7,8}. These and other previously reported methods only apply to certain regions of the skin; they do not readily allow thermal mapping measurement or determination of anisotropic properties and they operate effectively only in controlled, laboratory settings. Here, we introduce strategies that exploit ultrathin, soft systems⁹⁻¹⁸ of thermal actuators and sensors for robust, precise transport measurements, in a non-invasive manner that can rapidly capture both orientation and position dependent characteristics. Assessments of the skin at six different body locations in twenty-five human subjects illuminate systematic variations in both the thermal conductivity and thermal diffusivity, for which measurements by optical coherence tomography (OCT), and electrical impedance yield additional insights into the underlying physiology.

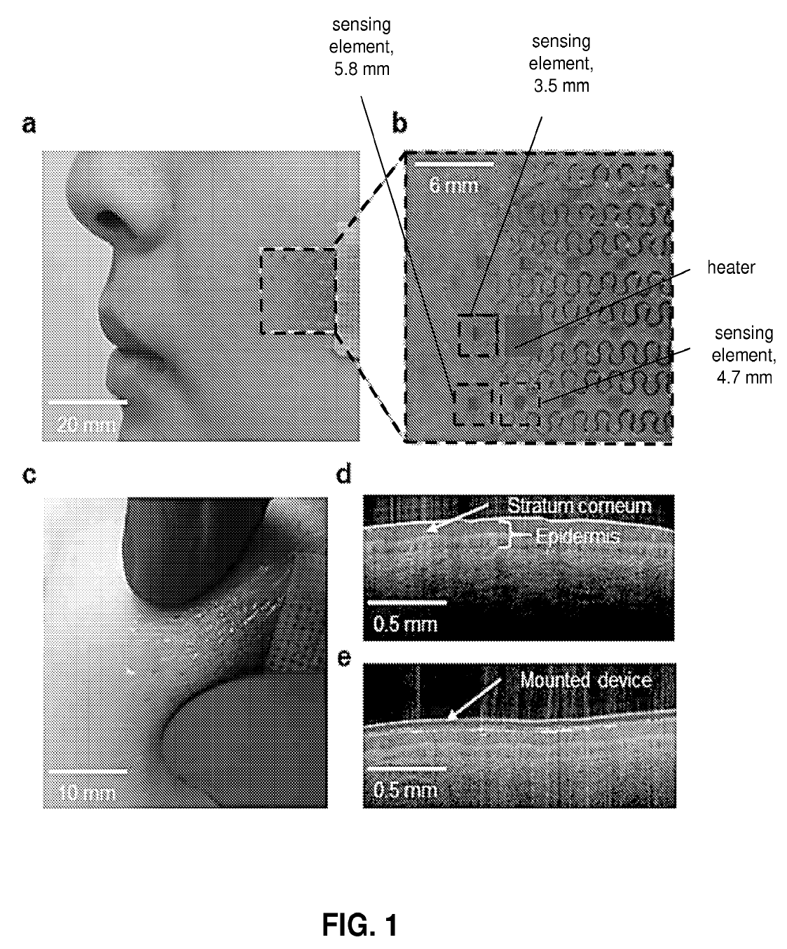

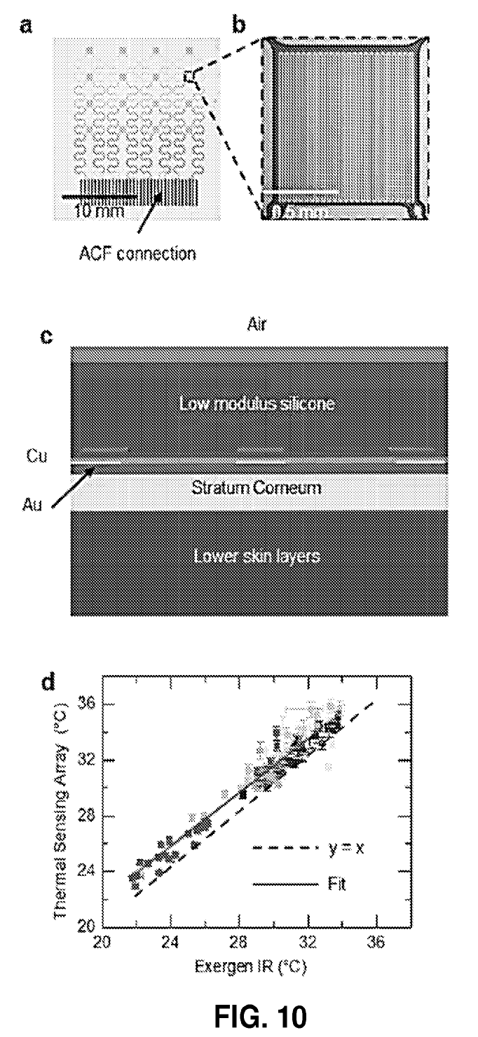

Our recent report¹⁰ introduced a type of thermal sensor with thickness, modulus and thermal mass matched to the epidermis, for spatiotemporal mapping of temperature on the surface of the skin with precision equal to or better than that of state-of-the-art infrared thermography systems. In the present work, advanced versions of this technology enable mapping of not only temperature but also thermal transport properties, including thermal conductivity and thermal diffusivity (and, therefore, the heat capacity per unit volume via the ratio of these two quantities) and their in-plane directional anisotropies. A representative device, shown in FIG. 1, a and b, mounted on the cheek, consists of a 4×4 array of interconnected filamentary metal structures (Cr/Au; 6/75 nm thick, 10 μm wide) that simultaneously function as thermal sensors and actuators, where the temperature coefficient of resistance of the metal couples changes in temperature to changes in resistance. A thin (<3 μm) film of polyimide encapsulates these structures and their electrical interconnects (Ti/Cu/Ti/Au; 10/500/10/25 nm thick, 50 μm wide) both above and below. A low modulus (35 kPa), thin coating (as small as 5 μm) of a silicone elastomer (Ecoflex 00-30, Smooth-on, USA) provides a conformal, intimate thermal interface directly to the SC. This soft mode of contact, together with the stretchable construction of the overall system, allows for repeated cycles of application, operation and removal without adverse effect on the device or the skin. The maximum heating powers used in experiments reported here introduce readily measurable changes in the temperature at the surface of the skin, but at levels that lie below the human sensory threshold. Optical coherence tomographic (OCT; VivoSight, Michelson Diagnostics, UK) images (FIG. 1, c and d) of a region of the skin before and after mounting the device highlight the high level of conformal contact afforded by soft, compliant construction. A wired electrical interface to a USB-powered portable data acquisition system enables operation in non-laboratory settings. See Supplementary Notes 1-2 and FIGS. 10-13F for device fabrication and data acquisition details, and statistical analysis of in vivo device temperature readings compared to infrared techniques.

Results

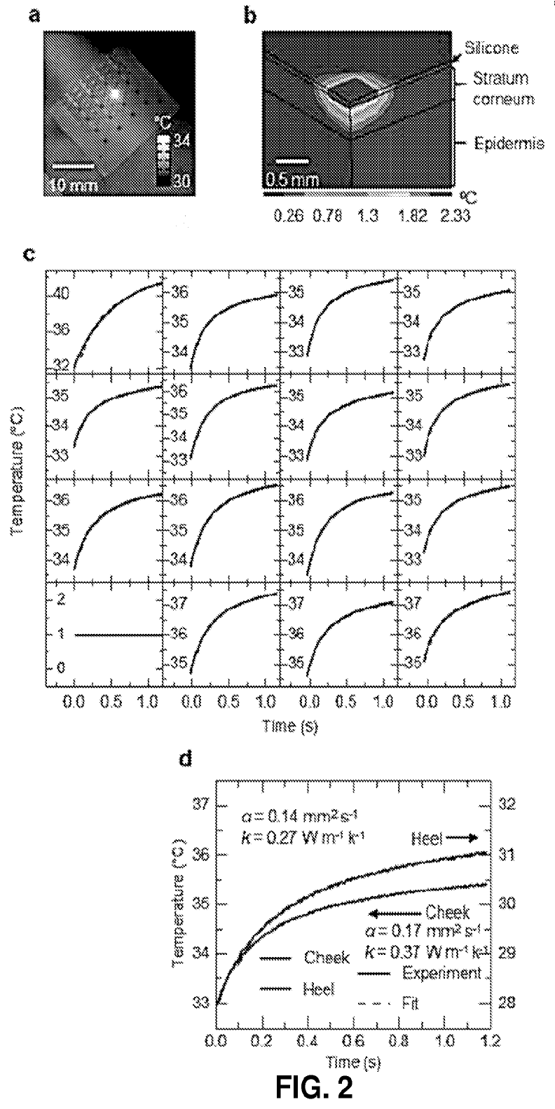

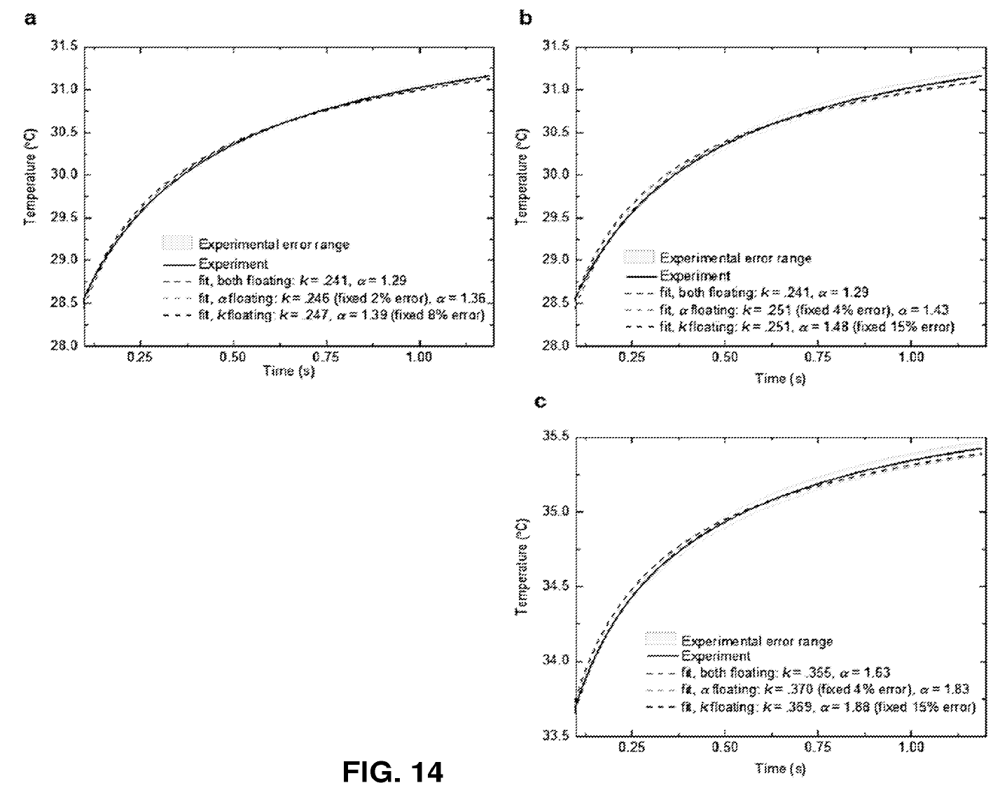

The sensors and actuators can be used interchangeably in two different modes to assess thermal transport. The first mode uses each element in the array sequentially and independently as both an actuator and a sensor. The measurement occurs quickly (<2 s), with capabilities for spatial mapping. An infrared image collected during the heating sequence (FIG. 2a) shows results of local, rapid heating generated by a single element. FIG. 2b illustrates findings from FEM modeling of the 3-dimensional temperature distribution after 1.2 s of heating, to provide a sense of the depth and lateral spatial scales associated with the measurement. For routine analysis, a simple analytical treatment in which the heating element is considered as a point heat source can be valuable. Here,

$$T = {T}_{∞} + {A}_{1} \frac{Q}{2 π {A}_{2} {k}_{skin}} erfc ( \frac{{A}_{2} \sqrt{{ρ}_{skin} {c}_{p , skin}}}{\sqrt{4 {k}_{skin} t}} ) \qquad ( 1 )$$

where T_{∞} is the temperature before heating, Q is the heating power, kₛₖᵢₙ is the thermal conductivity of the skin, ρₛₖᵢₙc_{p,skin} is the volumetric heat capacity of skin, t is time, erfc is the complementary error function, and A₂ represents an effective distance from the heater. A₁ is a parameter that accounts for details associated with the multilayered geometry of the device; its value is calibrated through measurements of materials with known thermal properties similar to those of the skin (water, ethylene glycol and polydimethylsiloxane). A₂ accounts for the fact that the thermal actuator (serpentine wire distributed over an area of 1×1 mm²) when used as a sensor records a temperature that corresponds to a weighted average over the area of the element. This average temperature, in the model of equation (1), is equivalent to the value at a distance A₂ away from an effective point source of heat. As a result, A₂ lies between 0 and 0.5 mm, depending on the geometric details and materials properties. In practice, A₂ is selected to yield quantitatively accurate results with materials of known thermal properties similar to those of skin. Analysis of in vivo data involves an iterative fitting procedure (Matlab, Mathworks, USA) to determine kₛₖᵢₙ and the thermal diffusivity (αₛₖᵢₙ=ρc_{p,skin}/kₛₖᵢₙ) using equation (1). Analysis of the sensitivity of the fitting process in the presence of experimental noise indicate maximum uncertainties of 2% and 8% for kₛₖᵢₙ and αₛₖᵢₙ, respectively (Supplementary Note 3 and FIG. 14. A similar analysis for errors in sensor calibration indicate maximum uncertainties of 5% and 15%. Measurements described subsequently demonstrate in vivo repeatability of better than 6% and 9% for kₛₖᵢₙ and αₛₖᵢₙ respectively. Comparison of thermal properties determined using equation (1) to those determined using solutions that explicitly integrate numerically over the sensor area indicate discrepancies that lie below the level of these experimental errors (Supplementary Note 4).

Examples of representative data (black lines) and calculations based on equation (1) (red dashed lines) for each element across the array appear in FIG. 2c. FIG. 2d presents similar results along with extracted values of kₛₖᵢₙ and αₛₖᵢₙ for the cheek and the heel pad. The differences between these two cases are significant, and likely result, at least in part, from the variations in the thicknesses of the SC, as described subsequently. The effective depth associated with the measurement can be approximated as¹⁹

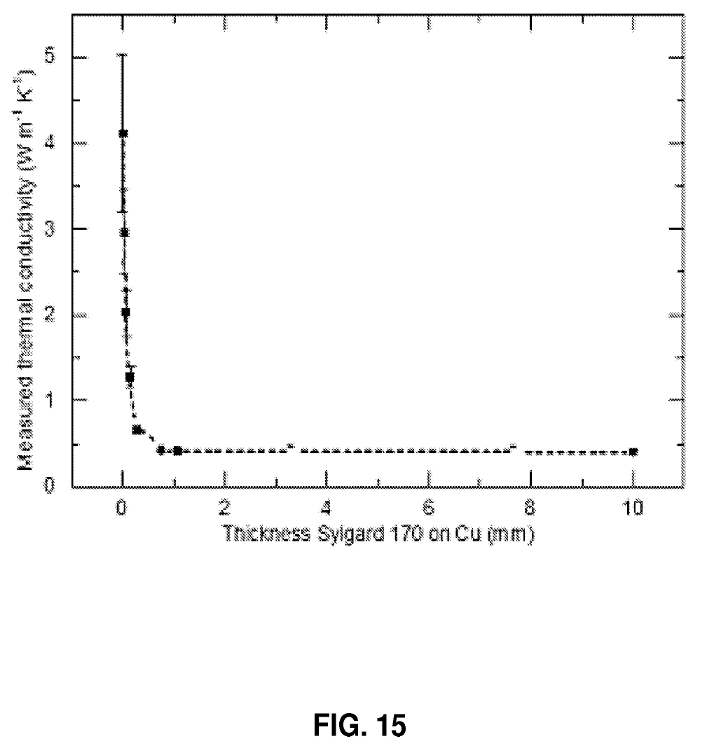

Δₚ=\sqrt{αtₘₐₓ} (2)

where tₘₐₓ is the characteristic measurement time. This equation gives a probing depth of ˜0.5 mm which agrees well with experimental analysis of measurement depth (Supplementary Note 5, FIG. 15) as well as the depth of heating shown by the FEM results in FIG. 2b. The depth dependent properties of the skin over this length scale influence the measurements.

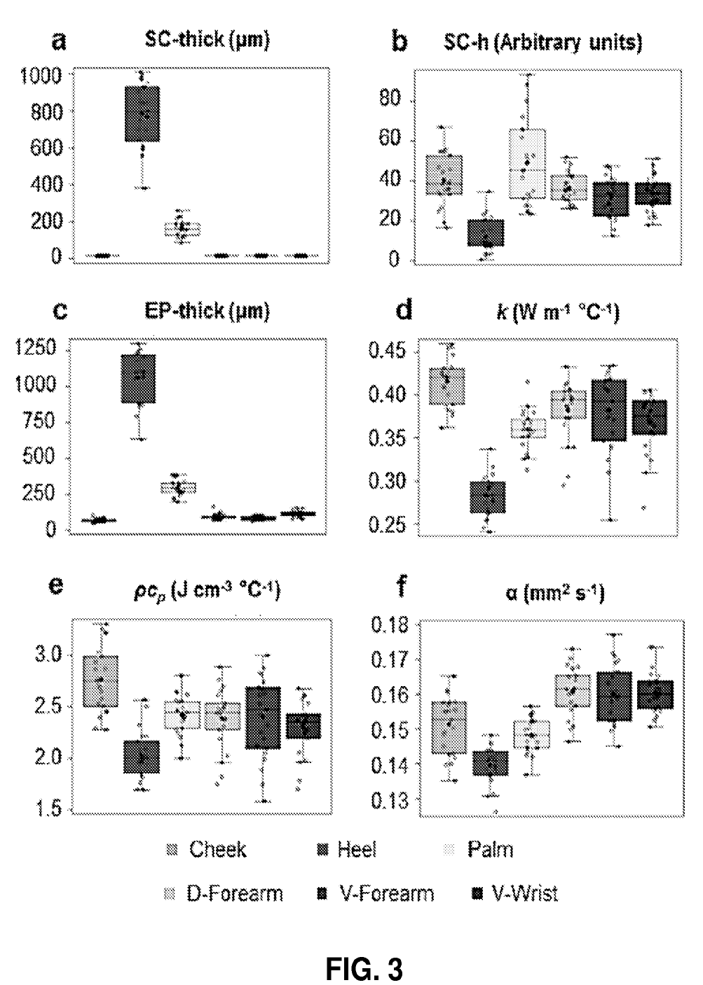

This measurement mode enabled comprehensive, systematic studies of thermal transport characteristics, in vivo, on twenty-five human subjects at six different body locations: cheek, dorsal forearm (d-forearm), volar forearm (v-forearm), volar wrist, palm and heel pad. Results for kₛₖᵢₙ and ρₛₖᵢₙc_{p,skin} follow from analysis using equation (1); αₛₖᵢₙ, which corresponds to their ratio, is useful to consider also, because it determines whether kₛₖᵢₙ and ρsₖᵢₙc_{p,skin} vary independently across body locations. Correlations between skin thermal properties to SC hydration measured using a corneometer (Cutometer® MPA 580, Courage+Khazaka Electronics GmbH), EP thickness and SC thickness measured using OCT provide further insights into the results. FIG. 3, which shows the distribution of these variables using a boxplot representation, reveals three distinct clusters for the thermal parameters: 1 cheek; 2 heel; and 3 palm, wrist, v-forearm and possibly d-forearm (the spread in the data here is relatively large due to the interference of hair on the measurement). Some separation occurs between the palm and the wrist/v-forearm/d-forearm, but to a degree that is not apparent from the univariate descriptive analysis. OCT yielded accurate values of SC thickness for the palm and heel pad but not for the follicular regions, where previous studies indicate a typical value of ˜15 μm²⁰⁻²².

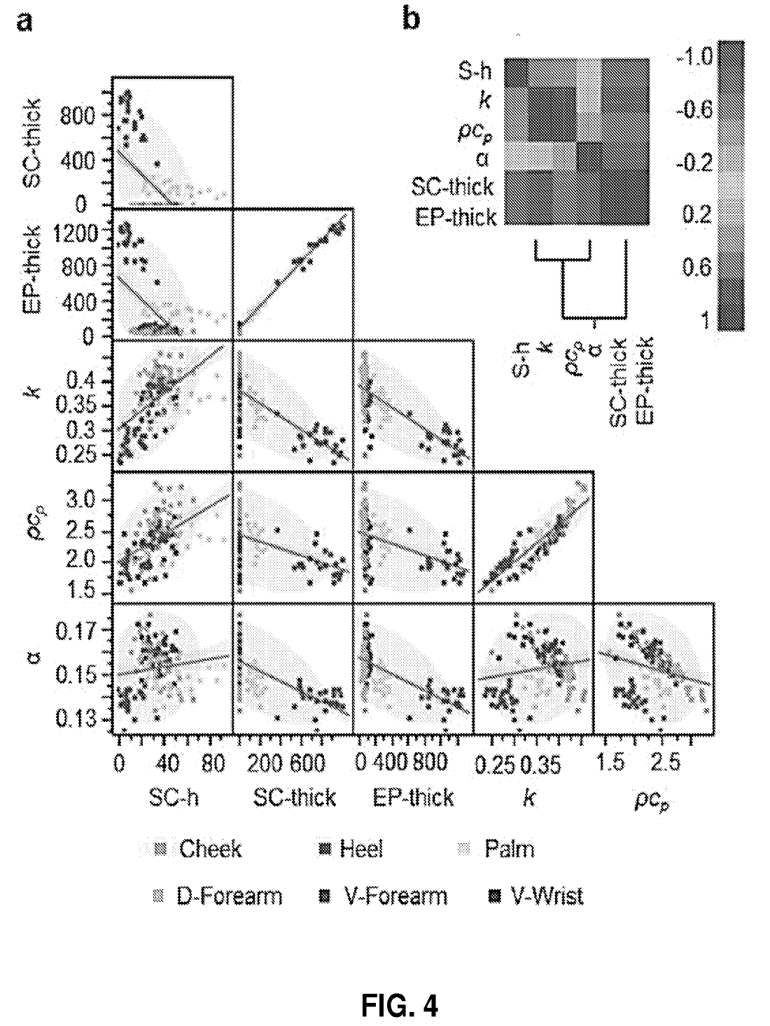

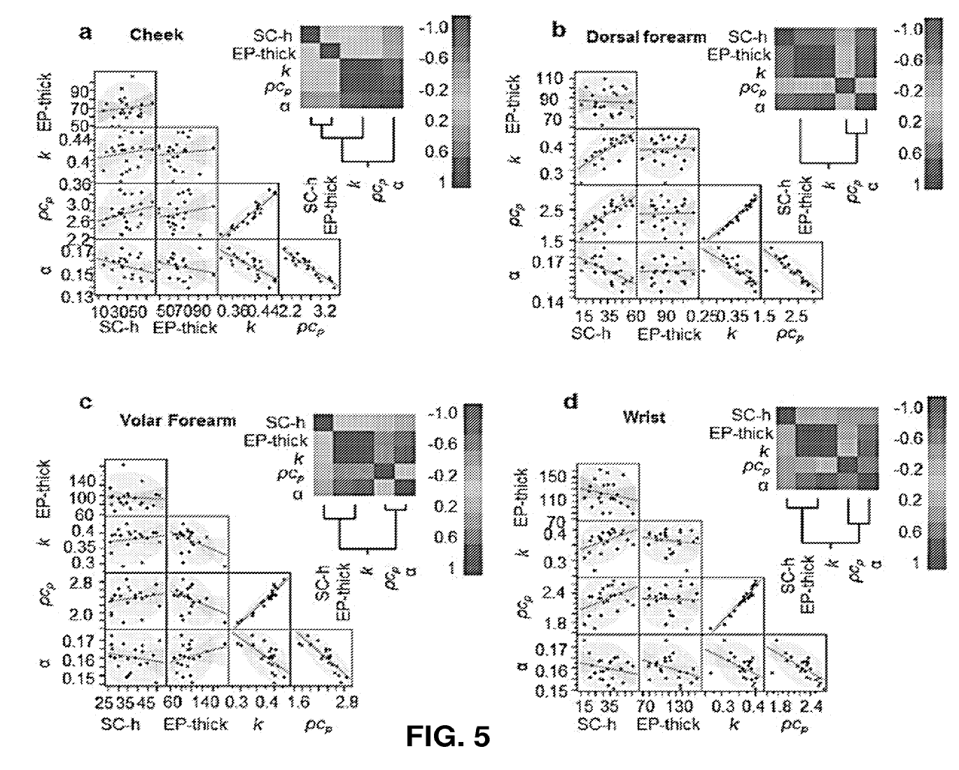

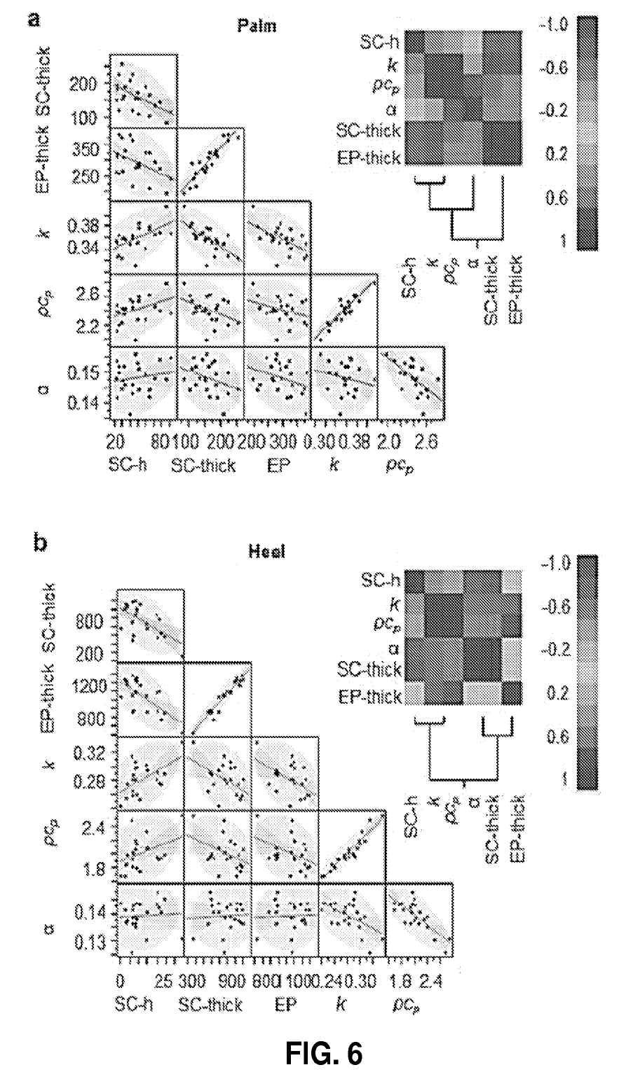

Pairwise correlation analyses for the skin thermal parameters, SC and EP thickness, and SC hydration appear in FIG. 4 for the entire data set, in FIG. 5 for each follicular region and in FIG. 6 for the palm and heel pad. The data show strong positive correlation between SC hydration and kₛₖᵢₙ and ρₛₖᵢₙc_{p,skin}. The ratio αₛₖᵢₙ exhibits a positive, but weaker, correlation with SC hydration. The data also indicate a strong negative correlation between SC/EP thickness and all three thermal properties (kₛₖᵢₙ, ρₛₖᵢₙc_{p,skin} and αₛₖᵢₙ). The EP thickness correlates with the SC thickness. SC is a significant fraction of the EP, especially in palmar regions, i.e. palm and heel pad. The SC thickness and SC hydration of the palmar regions show negative correlation. The strength of correlation depends strongly on body location (FIGS. 5 and 6, and Table 2).

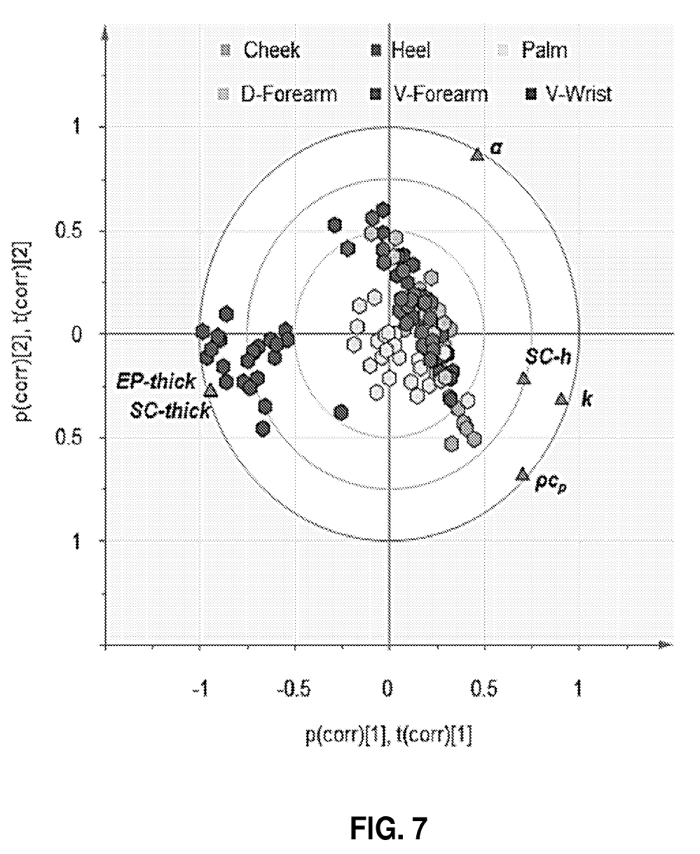

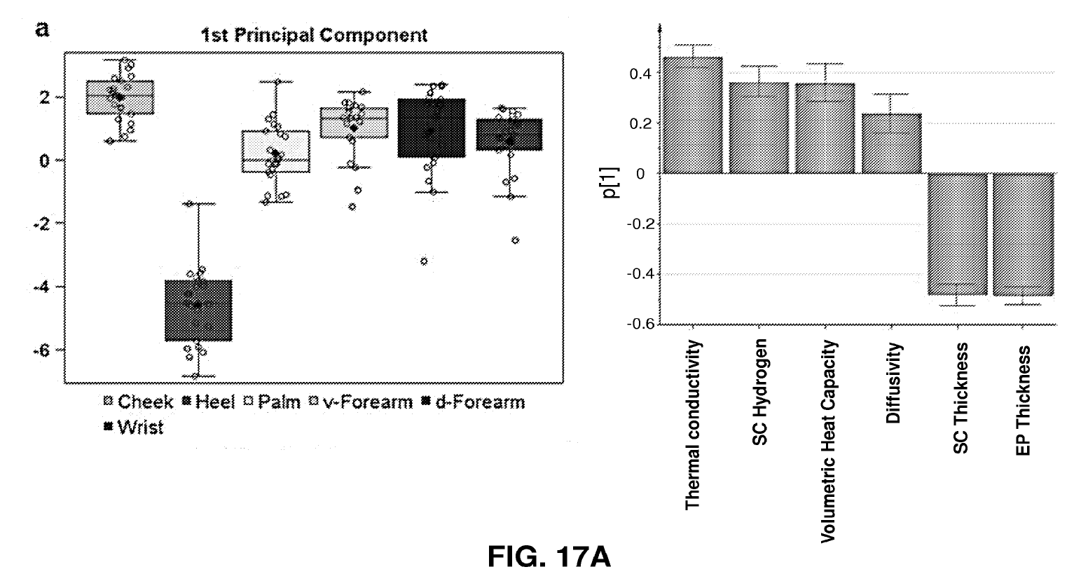

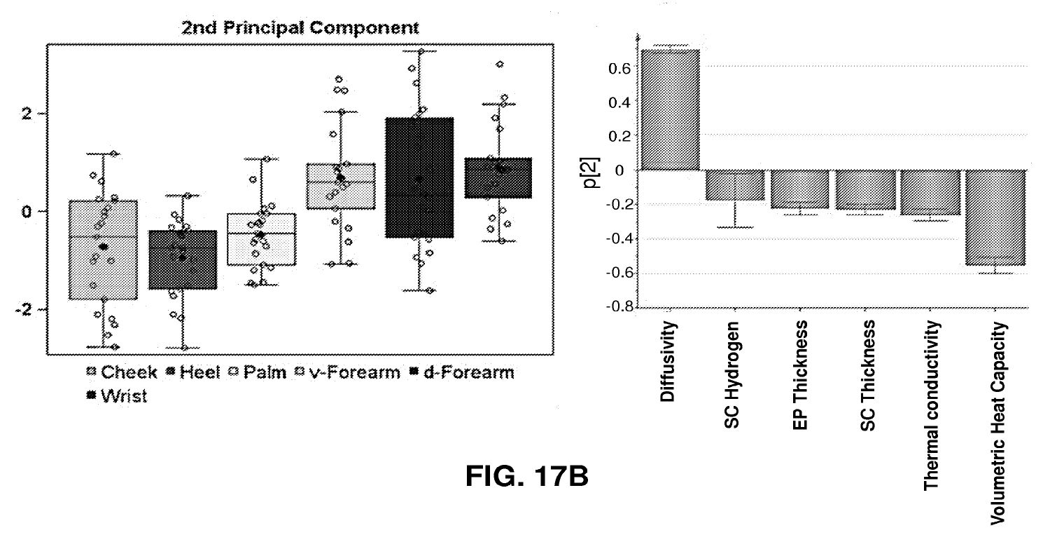

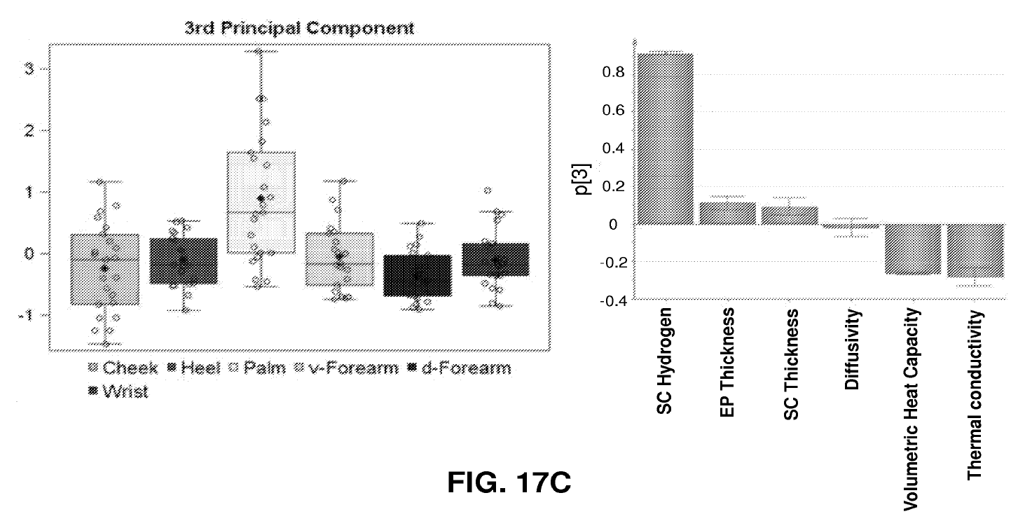

Principal component analysis (PCA), as a global multivariate approach of correlation analysis, appears in FIG. 7 and FIG. 17A-17C. PCA offers a graphical representation of both individuals and descriptors, with an ability to reveal hidden patterns in the data. The eigenvalues show that the first PCA axis explains 71% of the variance. The second and third components correspond to 20% and 7%, respectively. Hence, three components explain 97% of the inertia. In the biplot representation, the data, by location, are represented using scores coordinates, where the scores are the Principal Components (PCs). The first PC mainly separates observations of the heel from the other body areas (FIG. 7 and FIG. 17A). Discrimination also occurs, to a lesser extent, between the cheek and a group composed of palm, v-forearm, d-forearm and wrist (FIG. 17A). The second PC discriminates the arm and wrist location from the others (FIG. 17B). The third PC differentiates the palm (FIG. 17C). Based on the PCs, four distinct clusters occur within the data set: heel, cheek, palm, and wrist/v-forearm/d-forearm indicating four distinct locations with different physical properties. Descriptors close together on the biplot are highly correlated and conversely descriptors opposed are highly anti-correlated. On the biplot, SC hydration, thermal conductivity and volumetric heat capacity form one group and EP thickness and SC thickness form another with the two groups opposed on the first axis. This conveys the strong positive correlation of descriptors from the same group and conversely the negative correlation of descriptors from different groups. Interestingly, the thermal diffusivity is more linked to the second axis, and therefore quite independent to the other descriptors. This is consistent with previous remarks based on Pearson correlation coefficients.

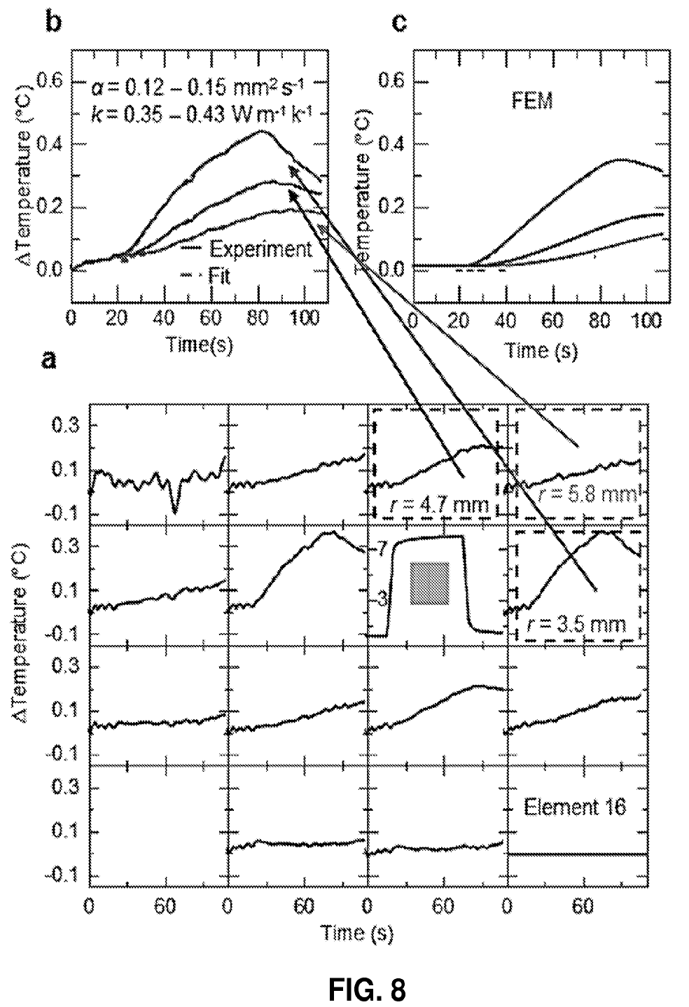

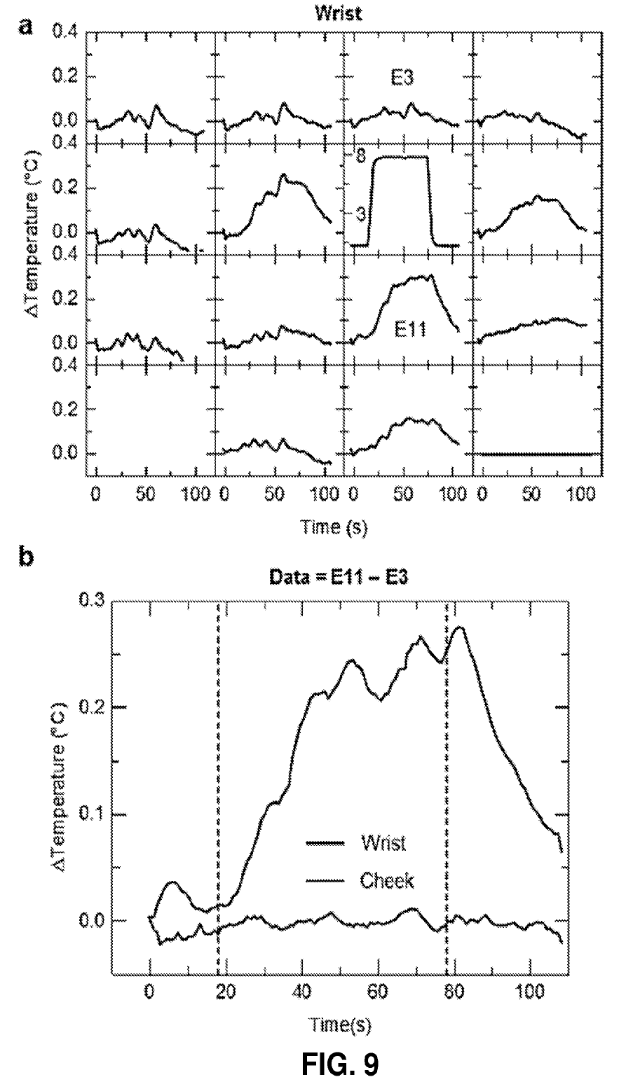

In addition to intrinsic properties of the skin itself, a second mode for characterizing thermal transport allows investigation of directional anisotropies and other effects related, for example, to blood flow in near surface arteries and veins. Here, application of electrical power (8 mW/mm² for 60 s) to a selected element (highlighted by the red box in FIG. 2b (optical image) and FIG. 8a (data)) introduces a controlled level of heating while the temperature of this element and all others in the array are simultaneously recorded as a function of time. Processing the data with an adjacent-averaging filter (window size=8 s), and subtracting the response of the sensor furthest from the actuator (Element 16) from that of each of the other sensors in the array minimizes effects of fluctuations in the ambient temperature. Here, the actuator can be approximated as a point source of heat, such that the transient temperature profile at a distance r can be written

$$T = {T}_{∞} + {A}_{1} \frac{Q}{2 π r ( t ) {k}_{skin}} erfc ( \frac{r ( t ) \sqrt{{ρ}_{skin} {c}_{p , skin}}}{\sqrt{4 {k}_{skin} t}} ) \qquad ( 3 )$$

where T_{∞} is the temperature before heating, Q is the heating power, kₛₖᵢₙ is the thermal conductivity of the skin, ρₛₖᵢₙc_{p,skin} is the volumetric heat capacity of skin, t is time, and erfc is the complementary error function. A₁ is a parameter that accounts for details associated with the multilayered geometry of the device; its value is calibrated through measurements of materials with known thermal properties similar to those of the skin (water, ethylene glycol and polydimethylsiloxane). r(t) represents the effective distance of the sensor from the heating element and takes the form of a time dependent function that accounts for the finite spatial area of the sensing element (Supplemental Note 6). kₛₖᵢₙ and αₛₖᵢₙ can be determined in a iterative process similar to that used in equation (1). The treatment of r causes a maximum relative error of <2% in the determination of kₛₖᵢₙ and αₛₖᵢₙ compared to those values determined by integrating equation (3) over its area at each time point (Supplemental Note 6). Representative results for different sensors appear in FIG. 8b. Finite element modeling (FEM) of the full device construct on a bilayer model of the skin yields temperature profiles (FIG. 8c) that closely match those observed in experiment. This measurement configuration provides additional information beyond that determined in equation (1) in the form of anisotropy in heat transport, at the expense of precision in the determination of thermal properties. FIG. 8 is an example of a skin area where the heat transport is strongly isotropic, while FIG. 9 illustrates the spatial changes in thermal transport on an area of skin with a significant anisotropic component to heat transport. Convective effects associated with blood that flows through vessels near the skin surface can induce in-plane, directional anisotropies in heat transport characteristics. FIG. 9 illustrates the effect when a device mounted on the volar aspect of the wrist includes a thermal actuator located over a near surface vein. The spatiotemporal temperature map in FIG. 9a shows a significantly larger increase in temperature at the sensor located downstream (more proximal to the body, labeled E11) from the actuator, compared the one upstream (more distal to the body, labeled E3), relative to the direction of blood flow. FIG. 9b highlights this difference through plots of the response of E3 subtracted from that of E11 for the case on the wrist, and of isotropic data from a representative case on the cheek. The degree of anisotropic transport varies in strength over the twenty-five subjects due to differences in the locations and sizes of blood vessels and their associated flow properties. Such measurement capabilities have relevance in the determination of cardiovascular health, through inferred measurements of blood flow, both naturally and in response to stimuli such as temporary occlusion.

### DISCUSSION

In summary, the work reported here reveals intrinsic thermal transport properties of the skin, including relationships to vascularization, blood flow, stratum corneum thickness and hydration level, made possible by expanded capabilities in soft ultrathin, non-invasive measurement systems that offer clear advantages compared to traditional approaches. Immediate further opportunities include use in studies of dermatological diseases, such as melanoma, rosacea and hyperpigmentation and their progression over time. The same techniques also offer the ability to examine the effectiveness of dermatologically active compounds. Wireless technology will provide a path to continuous monitoring of skin properties and function using these concepts.

Methods

Fabrication of Epidermal Thermal Sensing Array:

Fabrication begins with a 3″ Si wafer coated with a 200 nm layer of poly(methyl methacrylate), followed by 1 μm of polyimide. Photolithographic patterning of a bilayer of Cr (6 nm)/Au (75 nm) deposited by electron beam evaporation defines the sensing/heating elements. A second multilayer of Ti (10 nm)/Cu (500 nm)/Ti (10 nm)/Au (25 nm), lithographically patterned, forms the connections to sensing/heating elements and non-oxidizing bonding locations for external electrical connection. A second layer of polyimide (1 μm) places the sensing/heating elements in the neutral mechanical plane and provides electrical insulation and mechanical strain isolation. Reactive ion etching of the polyimide defines the mesh layout of the array and exposes the bonding locations. A watesoluble tape (3M, USA) enables removal of the mesh layout from the Si wafer, to expose its back surface for deposition of Ti (3 nm)/SiO₂ (30 nm) by electron beam evaporation. Transfer to a thin silicone layer (5 μm; Ecoflex, Smooth-On, USA) spin-cast onto a glass slide, surface treated to reduce adhesion of the silicone, results in the formation of strong bonds due to condensation reactions between exposed hydroxyl groups on the SiO₂ and silicone. Immersion in warm water allows removal of the tape. A thin (100 μm), flexible, conductive cable bonded with heat and pressure to contacting pads at the periphery serves as a connection to external electronics. A final layer of silicone (70 μm) in combination with a frame of medical tape (3M, USA) provides sufficient mechanical support to allow repeated (hundreds of times) use of a single device.

Experiments on Human Subjects:

The volunteers consisted of healthy females, age between 18 and 45 years old, with healthy, intact skin of type II-IV according to the Fitzpatrick classification, recruited by Stephens & Associates, TX, USA. The six investigational areas included the cheek, volar forearm, dorsal forearm, volar wrist, palm, and heel. Each subject acclimated to room temperature for 15 min immediately prior to measurement. The investigational areas were then gently cleaned with isopropyl alcohol, water, and dried with a swab to avoid skin irritation. Pictures were taken before and after the experimental procedures. SC hydration measurements used a 3 Cutometer® MPA 580 (Courage+Khazaka Electronics GmbH). Skin temperature was evaluated using a handheld IR thermometer (DermaTemp, Exergen Co., USA). Calibration of the experimental measurement system introduced here occurred at a single temperature point (room temperature). Evaluations involved lamination of the device onto the investigational area, collection of relevant data, followed by removal. Three additional corneometer readings were then collected, followed by measurements by optical coherence thomograpy (VivoSight, Michelson Diagnostics, UK).

Statistical Analyses:

Box plot representations (SAS statistical software release 9.3. SAS Institute Inc., Cary, N.C., USA) illustrate variables and trends by body location. The pairwise Pearson correlation coefficients were displayed as tables, scatterplot matrices, or heat map representations using JMP statistical software release 10.0 (JMP is a trademark of SAS Institute). Principal Component Analysis served as a global multivariate approach with a biplot representation of individuals and descriptors (SIMCA statistical software release 13.0, UMETRICS, Umeå, Sweden).

### REFERENCES

1 Graffe, K. V. D. Human Anatomy. 6th edn, 105 (McGraw Hill, 2001).
2 El-Brawany, M. A. et al. Measurement of thermal and ultrasonic properties of some biological tissues. Journal of Medical Engineering and Technology 33, 249-256 (2009).
3 Werner, U., Giese, K., Sennhenn, B., Plamann, K. & Kolmel, K. Measurement of the thermal diffusivity of human epidermis by studying thermal wave propagation. Physics in medicine and biology 37, 21-35 (1992).
4 Togawa, T. & Saito, H. Non-contact imaging of thermal properties of the skin. Physiological Measurement 15, 291-298 (1994).
5 Ducharme, M. B. & Tikuisis, P. In vivo thermal conductivity of the human forearm tissues. Journal of Applied Physiology 70, 2682-2690 (1991).
6 Jin, C. et al. A feasible method for measuring the blood flow velocity in superficial artery based on the laser induced dynamic thermography. Infrared Physics and Technology 55, 462-468 (2012).
7 Arnaud, F. et al. A micro thermal diffusion sensor for non-invasive skin characterization. Sensors and Actuators: A. Physical 41, 240-243 (1994).
8 Raamat, R., Jagomägi, K. & Kingisepp, P. H. Simultaneous recording of fingertip skin blood flow changes by multiprobe laser Doppler flowmetry and frequency-corrected thermal clearance. Microvascular research 64, 214-219 (2002).
9 Kim, D. H. et al. Epidermal Electronics. Science 333, 838-843, doi:DOI 10.1126/science.1206157 (2011).
10 Webb, R. C. et al. Ultrathin conformal devices for precise and continuous thermal characterization of human skin. Nat Mater 12, 938-944, doi:Doi 10.1038/Nmat3755 (2013).
11 Wang, S. et al. Mechanics of Epidermal Electronics. Journal of Applied Mechanics 79, 031022-031022, doi:10.1115/1.4005963 (2012).
12 Huang, X. et al. Epidermal impedance sensing sheets for precision hydration assessment and spatial mapping. IEEE transactions on bio-medical engineering 60, 2848-2857, doi:10.1109/TBME.2013.2264879 (2013).
13 Kaltenbrunner, M. et al. An ultra-lightweight design for imperceptible plastic electronics. Nature 499, 458-463, doi:10.1038/nature12314 (2013).
14 Someya, T. et al. Conformable, flexible, large-area networks of pressure and thermal sensors with organic transistor active matrixes. Proceedings of the National Academy of Sciences of the United States of America 102, 12321-12325, doi:10.1073/pnas.0502392102 (2005).
15 Mannsfeld, S. C. et al. Highly sensitive flexible pressure sensors with microstructured rubber dielectric layers. Nat Mater 9, 859-864, doi:10.1038/nmat2834 (2010).
16 Lacour, S. P., Jones, J., Suo, Z. & Wagner, S. Design and performance of thin metal film interconnects for skin-like electronic circuits. Ieee Electr Device L 25, 179-181, doi:Doi 10.1109/Led.2004.825190 (2004).
17 Sun, J. Y. et al. Inorganic islands on a highly stretchable polyimide substrate. J Mater Res 24, 3338-3342, doi:Doi 10.1557/Jmr.2009.0417 (2009).
18 Li, T., Huang, Z. Y., Suo, Z., Lacour, S. P. & Wagner, S. Stretchability of thin metal films on elastomer substrates. Appl Phys Lett 85, 3435-3437, doi:Doi 10.1063/1.1806275 (2004).
19 Gustafsson, S. E. Transient plane source techniques for thermal conductivity and thermal diffusivity measurements of solid materials. Review of Scientific Instruments 62, 797-804 (1991).
20 Robertson, K. & Rees, J. L. Variation in epidermal morphology in human skin at different body sites as measured by reflectance confocal microscopy. Acta dermato-venereologica 90, 368-373, doi:10.2340/00015555-0875 (2010).
21 Egawa, M., Hirao, T. & Takahashi, M. In vivo estimation of stratum corneum thickness from water concentration profiles obtained with Raman spectroscopy. Acta dermato-venereologica 87, 4-8, doi:10.2340/00015555-0183 (2007).
22 Bohling, A., Bielfeldt, S., Himmelmann, A., Keskin, M. & Wilhelm, K. P. Comparison of the stratum corneum thickness measured in vivo with confocal Raman spectroscopy and confocal reflectance microscopy. Skin research and technology: official journal of International Society for Bioengineering and the Skin 20, 50-57, doi:10.1111/srt.12082 (2014).

Supplementary Information: Thermal Transport Characteristics of Human Skin Measured In Vivo Using Ultrathin Conformal Arrays of Thermal Sensors and Actuators

Supplementary Note 1: Fabrication Procedure for Ultrathin Thermal Sensing Arrays

Prepare Polymer Base Layers
1. Clean a 3″ Si wafer (Acetone, IPA→Dry 5 min at 110° C.).
2. Spin coat with PMMA (poly(methyl methacrylate) 495 A2 (Microchem), spun at 3,000 rpm for 30 s.
3. Anneal at 180° C. for 1 min.
4. Spin coat with polyimide (PI, poly(pyromellitic dianhydride-co-4,4′-oxydianiline), amic acid solution, Sigma-Aldrich, spun at 4,000 rpm for 30 s).
5. Anneal at 110° C. for 30 s.
6. Anneal at 150° C. for 5 min.
7. Anneal at 250° C. under vacuum for 1 hr.

Deposit First Metallization
8. E-beam 6/75 nm Cr/Au.
9. Pattern photoresist (PR; Clariant AZ5214, 3000 rpm, 30 s) with 365 nm optical lithography through iron oxide mask (Karl Suss MJB3).
   Develop in aqueous base developer (MIF 327).
10. Etch Au with TFA Au etchant (Transene).
11. Etch Cr with CR-7 Cr Mask Etchant (Cyantek).
12. Remove PR w/ Acetone, IPA rinse.
13. Dry 5 min at 150° C.

Deposit Second Metallization
14. E-beam 10/500/10/25 nm Ti/Cu/Ti/Au.
15. Pattern PR AZ5214.
16. Etch Au with TFA Au etchant.
17. Etch Ti with 6:1 Buffered Oxide Etchant.
18. Etch Cu with CE-100 etchant (Transene).
19. Etch Ti with 6:1 Buffered Oxide Etchant.
20. Remove PR w/ Acetone, IPA rinse.
21. Dry 5 min at 150° C.

Isolate Entire Device
22. Spin coat with PI.
23. Anneal at 110° C. for 30 s.
24. Anneal at 150° C. for 5 min.
25. Anneal at 250° C. under vacuum for 1 hr.
26. Pattern photoresist (PR; Clariant AZ4620, 3000 rpm, 30 s) with 365 nm optical lithography through iron oxide mask (Karl Suss MJB3).
   Develop in aqueous base developer (AZ 400K diluted 1:3, AZ 400K:Water).
27. RIE (150 mTorr, 20 sccm O₂, 200 W, 20 min).

Release and Transfer
28. Release w/ boiling Acetone.
29. Transfer to water-soluble tape (Wave Solder Tape, 5414, 3M).
30. E-beam 3/30 nm Ti/SiO₂.
31. Transfer to ˜10 μm silicone sheet (Ecoflex, Smooth-on Co.) coated on silanized glass slide.
32. Immerse in warm water to dissolve tape.
33. Immerse quickly in Chrome Mask Etchant to remove any remaining residue.
34. Bond thin, flexible cable (Elform, HST-9805-210) using hot iron with firm pressure.
35. Apply additional silicone (10-100 μm) by doctor blade
36. Apply silicone medical tape frame (Ease Release Tape, 3M).
37. Remove device.

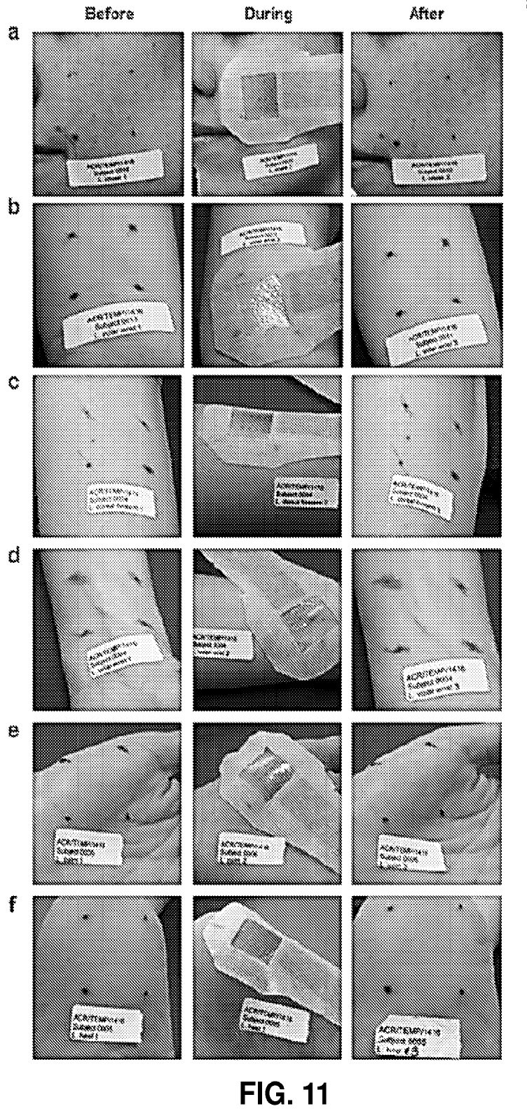

In order to provide a more appropriate system for repeated clinical use, the initially demonstrated system was improved upon in several ways. First, an electron beam evaporated metallic stack of Ti/Cu/Ti/Au (10/500/10/25 nm) replaced the expensive Au interconnect wiring system. This system provided the desired low resistivity interconnects while using minimal Au as a contact material. Narrow line widths (10 μm) in the sensing/heating elements provided high resistance in a small spatial area, shown in FIG. 10b, minimizing undesired heating in interconnect wires. A thin layer of Ecoflex (smooth-on, ETC) polymer between the sensor/heater elements (FIG. 10c) and the skin improved the adhesion directly between the heating element and the skin, minimizing errors in thermal transients that may be caused by air gaps. Finally, a silicone adhesive based tape (Ease Release, 3M, USA) functioned as a frame for the device, providing a flexible but robust mechanical support for repeated use over >100 applications (see FIG. 11 for images before, during, and after measurement on each body location in the clinical study). Finally, the data acquisition and control system was in the form of a low cost, USB-powered portable system for practical clinical use. High temperature resolution was achieved by the 22-bit digital multimeter (USB-4065, National Instruments, USA) and time-multiplexing was achieved by the use of a USB-powered, voltage isolated switch circuit (U802, Ledgestone Technologies LLC, USA).

Supplementary Note 2: Temperature Measurements Across all Body Locations

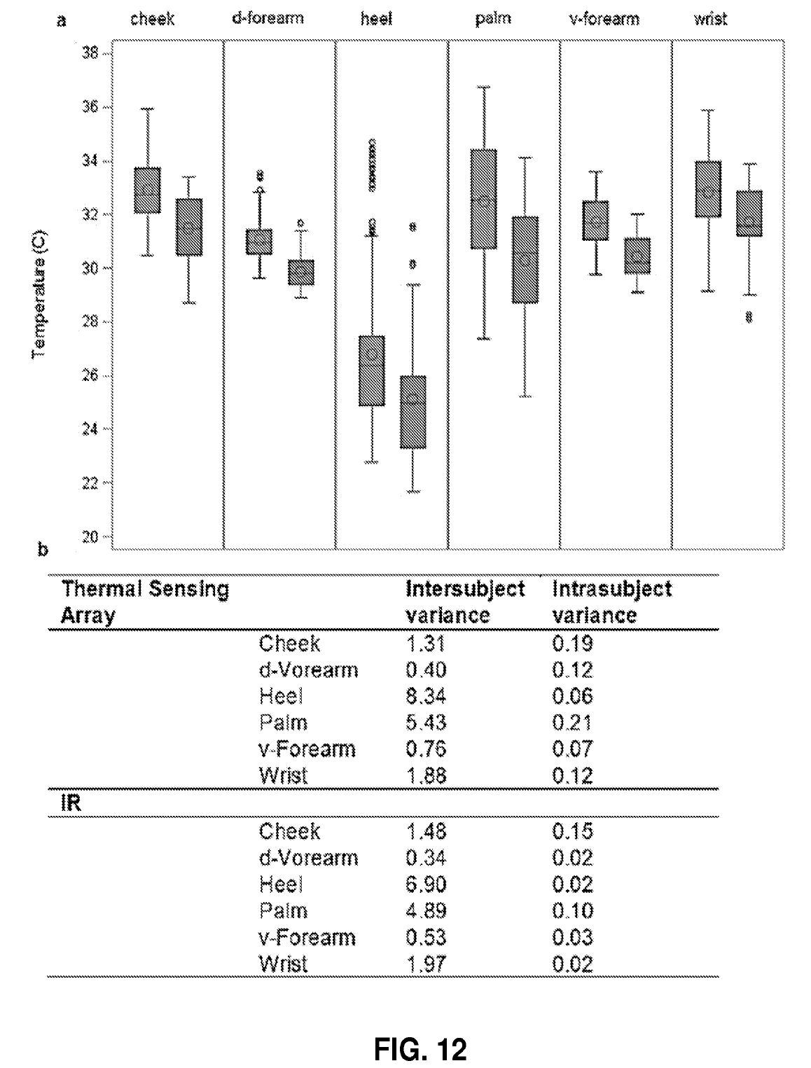

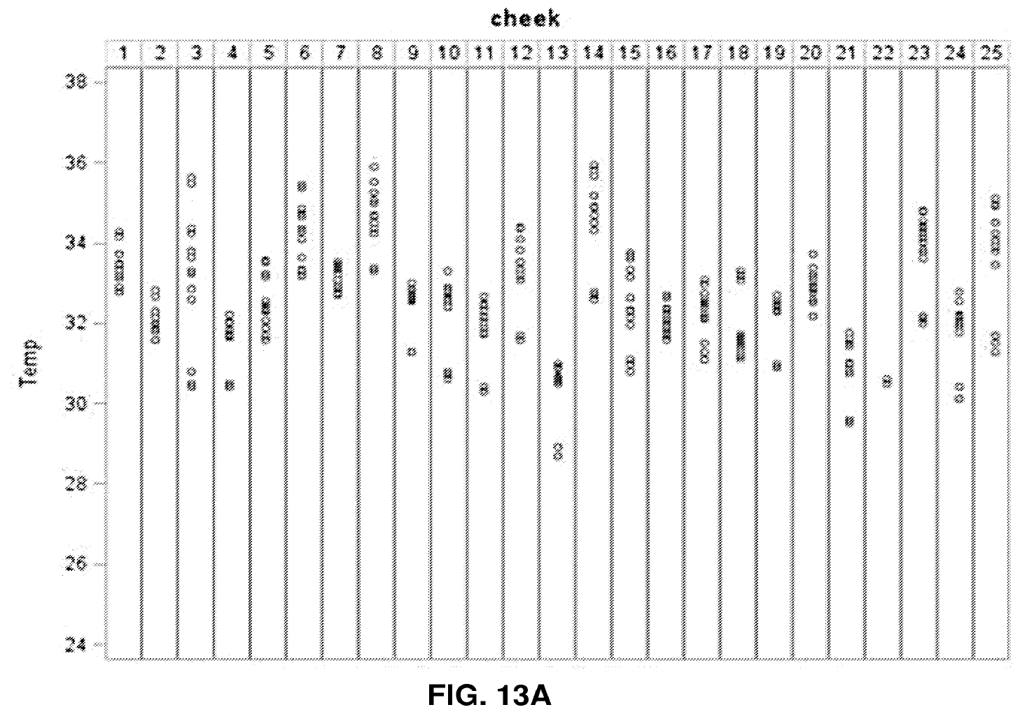

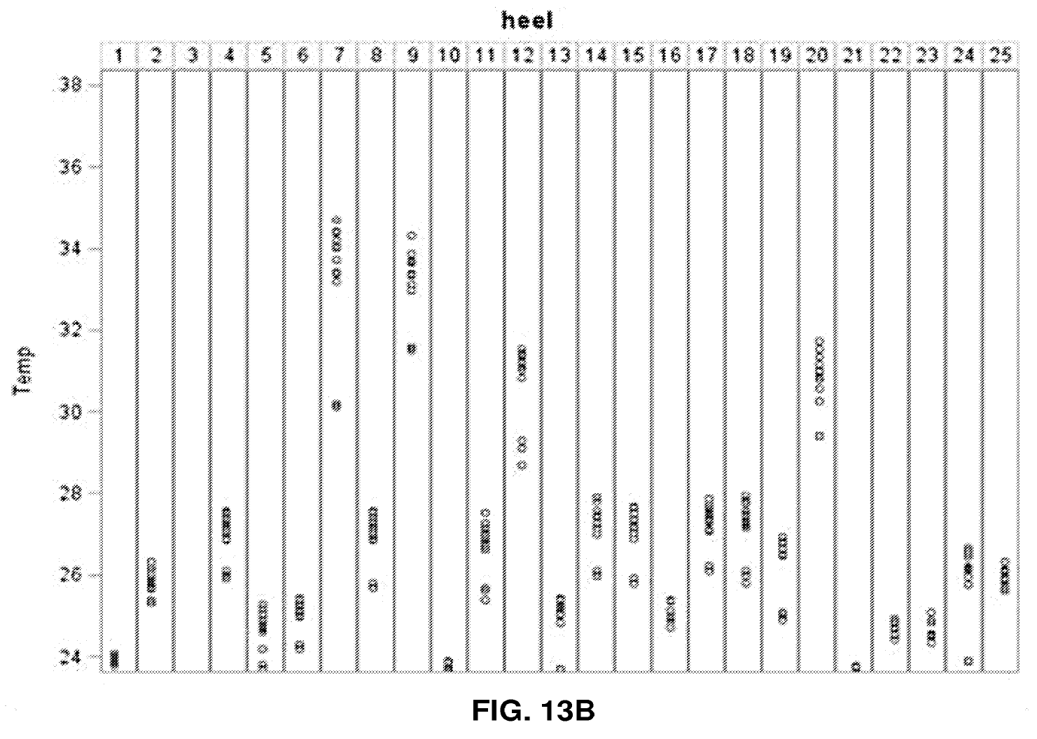

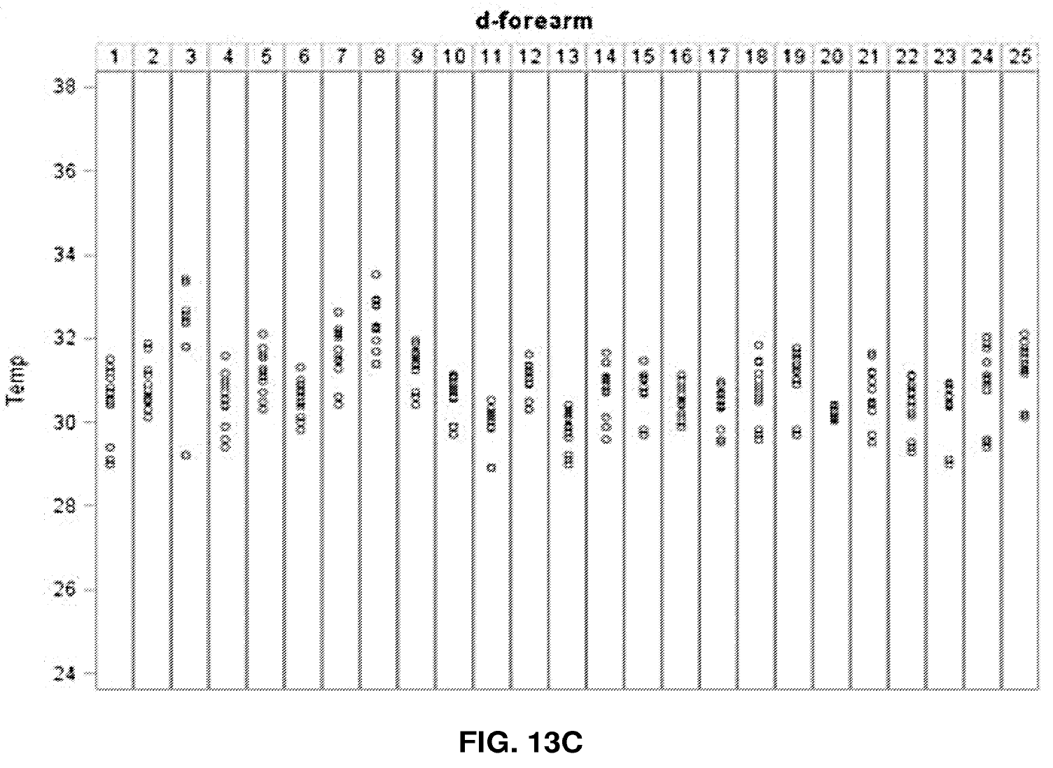

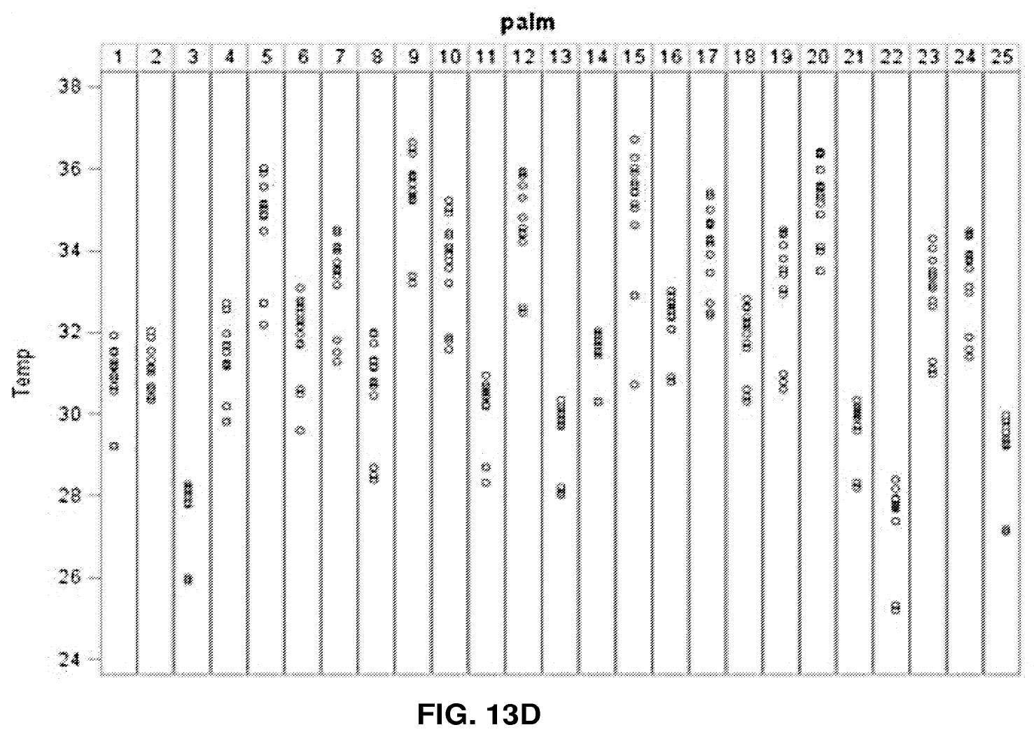

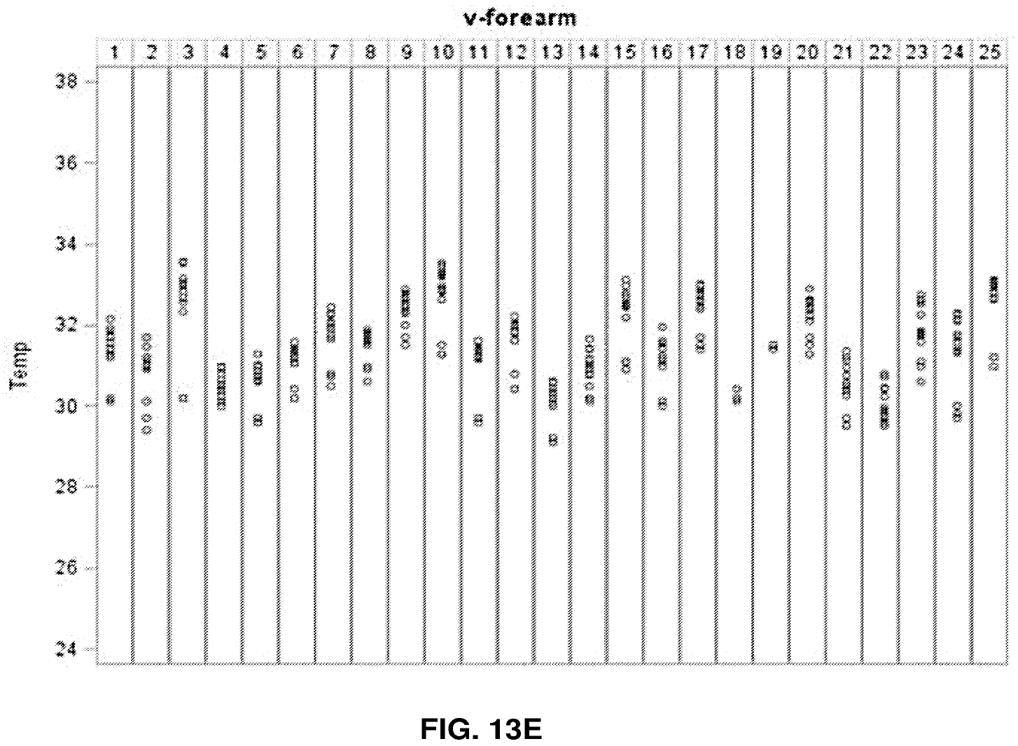

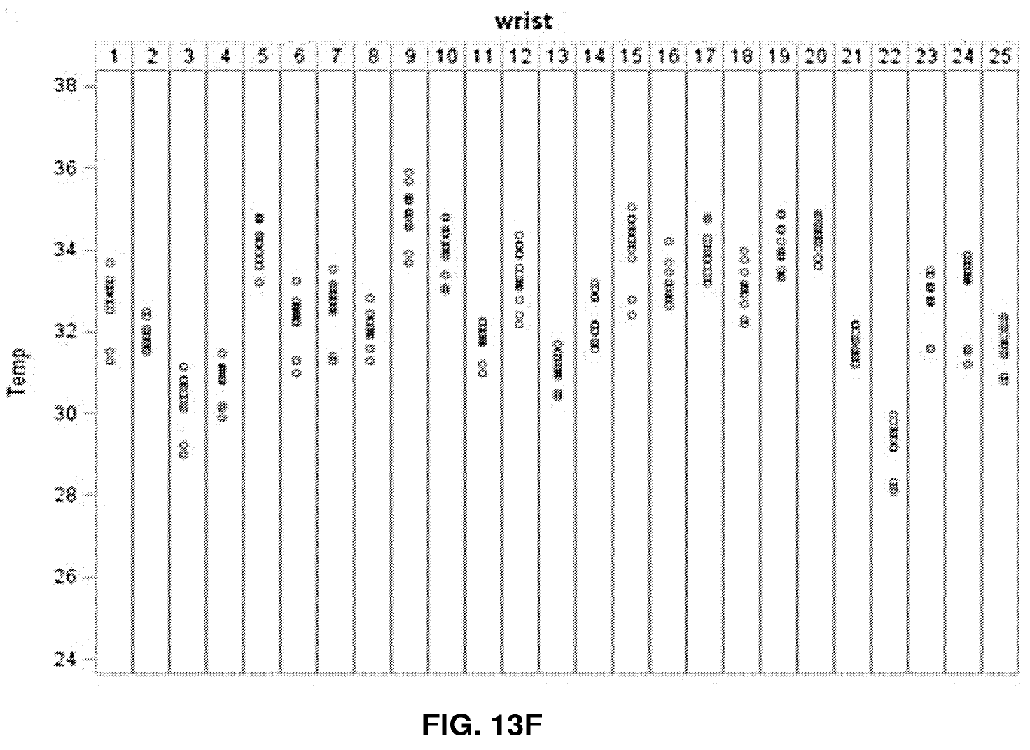

In order to verify temperature accuracy, temperature recordings by the device array are compared to recordings by a commercial infrared thermometer (DermaTemp, Exergen Co., USA) on each body location (FIG. 10d). The temperature values correlate well (Pearson's correlation coefficient, R, =0.98, slope=0.95±0.02, intercept=2.5±0.5, standard errors), verifying the value of the device in the context of epidermal temperature sensing across varied body locations, as demonstrated previously¹. Average temperature variations between body locations are shown in FIG. 12, and temperature variations measured on each body location on each subject are shown in FIGS. 13A-13F.

Supplemental Note 3: Estimated Error in Fitting Models for Clinical Study

The fitting model described by equation (1) and FIG. 2 is used to determine thermal property data for the 150 body locations measured during the clinical study. In this fitting procedure, two parameters, thermal conductivity and thermal diffusivity, are fit simultaneously. We assess the potential error in this fitting procedure by fixing one of the parameters, and allowing the other to float to determine the best fit with experimental data. In order to determine the fixed parameter value, we initially conduct the fit with both parameters floating to determine the best fit with experimental data (FIG. 14, red dashed line). We then fix one parameter, with a relative error from the best fit value, and allow the second parameter to float to determine a new best fit. We increase the error introduced to the fixed parameter until the new best fit curve falls just outside the error range of the experimental data (FIG. 14; best fit curves after applying error shown as blue and green dashed line; error range of experimental data shaded in red). The error range associated with the precision (i.e. the sensitivity of measurements using the same device one measurement to the next) of experimental data (FIG. 13A) is given as ±0.04° C., which is >3σ, where σ=0.013° C. is the in vivo experimental standard deviation of error from the mean. This error analysis conducted on several sets of in vivo data from our clinical study results in 2-3% potential error in the value of k and 8% potential error in the value of a, with representative analyses from the heel shown in FIG. 14a. Each in vivo measurement involves solutions to k and a from each of fifteen sensors in the array. The average standard deviation across all body locations, excluding the dorsal forearm which has large deviations due to hair on some subjects, of all subjects is 6% (0.02 W m⁻¹ K⁻¹) and 9% (0.013 mm² s⁻¹) for k and α respectively.

The error range associated with the sensor accuracy (i.e. the reliability of measurements when using different devices one measurement to the next) of experimental data is given by the 95% confidence interval of the sensor calibration of temperature sensitivity. This error analysis conducted on several sets of in vivo data from our clinical study results in 4-5% potential error in the value of k and 15% potential error in the value of α, with representative analyses from the heel and cheek shown in FIGS. 14b and 14c respectively.

Supplemental Note 4: Error Analysis of Equation (1) Approximations

The algorithm used to calculate skin thermal transport properties from transient heating in individual elements, shown in equation (1), is a convenient approximation to the solution of the average temperature of a small square with finite dimensions during transient heating. The approximation in equation (1) assumes that the average temperature in the square can be approximated by assuming a point heat source at the center of the square, and a temperature rise some distance A₂ away from the point source. The iteration of equation (1) is computationally inexpensive, which allows for rapid computation of the data from each element in the array. The potential error associated with equation (1) is investigated by comparison to the more exact, and computationally expensive, solution given by Gustafsson²

$$\overline{Δ T ( τ )} = \frac{{P}_{0} H ( τ )}{4 {π}^{\frac{1}{2}} bk} \qquad ( S1 )$$

where P₀ is the power output of the heater, b is the half width of the square heating element (0.5 mm for the device), k is the thermal conductivity,

$$τ = \frac{t α}{{b}^{2}} \qquad ( S2 )$$

where α is the thermal diffusivity, t is time and

H(τ)=∫₀^{τ}dν{erf(ν⁻¹)−π^{-1/2}ν[1−exp(−ν⁻²)]}² (S3)

where erf is the error function given by

erf(x)=2π^{-1/2}∫₀^{x}dνexp(−ν²). (S4)

equation (S1) accounts for the finite spatial extent of the heater to determine the average measured temperature of the heater. However, iterating the solutions of equations (S1)-(S4) over the large body of data with the high frequency measurement of data across many elements in an array quickly becomes computationally intensive. In order to compare the error using equation (1), we compare the thermal properties, k and a, determined on a representative dataset using equation (1) to those determined by the iteration procedure of equations (S1)-(S4), once calibrated with known calibration media (water and ethylene glycol). The average discrepancy between the two procedures in the solution for k and a is 3% and 8%, respectively, which is within the previously described error ranges due to noise. These potential errors will manifest in the form of constant accuracy offset that will be consistent across all devices. As a result, these potential errors will not influence the precision between measurements, different devices or the resultant correlation statistics that are of primary interest.

Supplemental Note 5: Estimation of Measurement Depth

The measurement technique outlined by equation (1) results in thermal property values that are a weighted average of the values encountered through the depth of skin that is probed by the measurement. The measurement depth can be approximated by equation (2), which results in a measurement depth of ˜500-1000 μm in skin. We verify this result experimentally by conducting measurements on varying thickness of a polymer, with thermal properties similar to skin (Sylgard 170, Dow Corning, USA), on a base substrate of copper. The copper acts a thermal ground plane that will result in rapidly increasing measured thermal properties as the measurement depth approaches the polymer thickness. The resultant measured thermal conductivities on various thicknesses of polymer on copper are shown in FIG. 15, and the measured thermal conductivities begin to rise rapidly at a polymer thickness of approximately 500 μm.

Supplemental Note 6: Error Analysis of Equation (3) Approximations

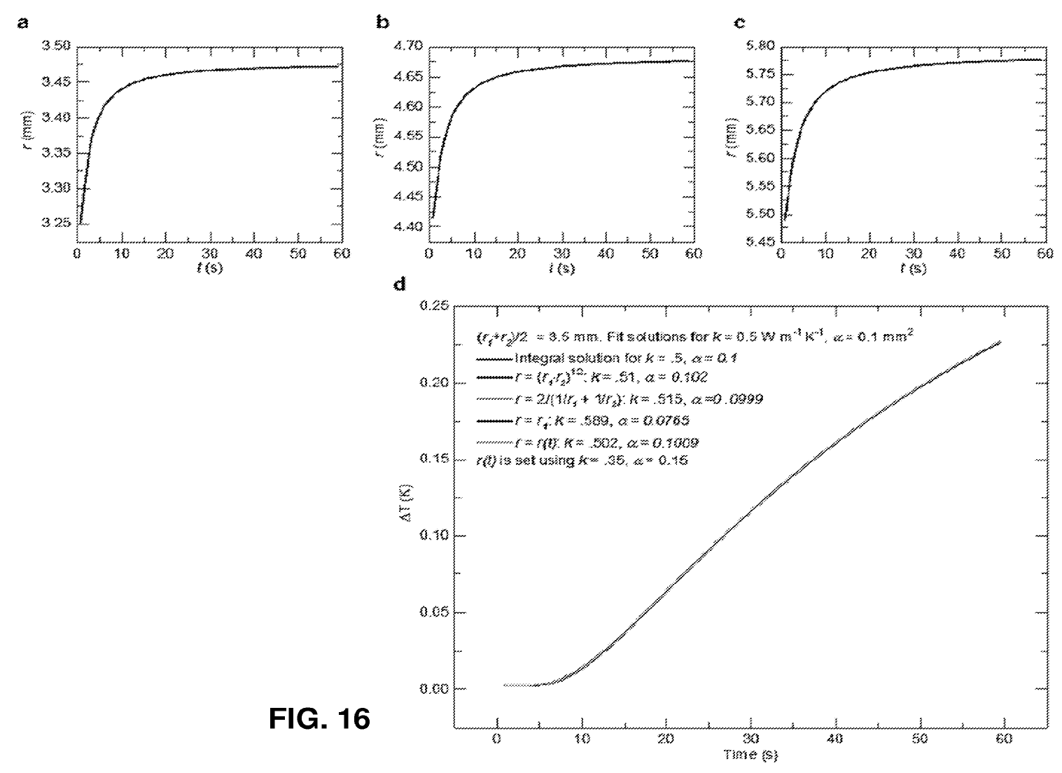

The measurement configuration outlined by equation (3) and FIG. 8 assumes a discrete distance, r, away from a point source heater. The sensors in the array in use here have a finite aerial spatial extent of 1 mm×1 mm, with <3 μm thickness. The temperature increase recorded by a sensor corresponds to the average temperature increase over the sensor area. Assuming isotropic radial conduction, valid for cases without anisotropic convective transport due to blood, the average temperature across the sensor, T̅, is approximately equal to the average temperature rise between points r₁ and r₂ away from a point source heater, given by

$$\overline{T} = \frac{{\int }_{{r}_{1}}^{{r}_{2}} \frac{Q}{2 π {rk}_{skin}} erfc ( \frac{r \sqrt{{ρ}_{skin} {c}_{p , skin}}}{\sqrt{4 {k}_{skin} t}} ) dr}{{r}_{2} - {r}_{1}} . \qquad ( S5 )$$

Where r₁ and r₂ are 1 mm apart and represent the distances of the sensor near and far edges, respectively, from the heater, equation (S5) can be approximated by

$$\overline{T} = \frac{Q}{2 π r ( t ) {k}_{skin}} erfc ( \frac{r ( t ) \sqrt{{ρ}_{skin} {c}_{p , skin}}}{\sqrt{4 {k}_{skin} t}} ) \qquad ( S6 )$$

where the integral average over the sensor in equation (S5) has been replaced by r(t), a time dependent characteristic distance. r(t) is determined numerically by setting equation (S5) equal to equation (S6). Specifically, equation (S5) is solved for a fixed kₛₖᵢₙ and ρₛₖᵢₙc_{p,skin}. equation (S6) is then solved in an iterative fashion to minimize the error between equation (S6) and equation (S5), where r(t) is allowed to vary, and kₛₖᵢₙ and ρₛₖᵢₙc_{p,skin} are fixed to the values used in the solution for equation (S5). kₛₖᵢₙ=0.35 W m⁻¹ K⁻¹ and ρₛₖᵢₙc_{p,skin}=2.33 J cm⁻³K⁻¹ are the approximate midpoint values of the in vivo data, and are used to establish r(t) for the three sensor distances of ˜3.5 mm, ˜4.7 mm, and ˜5.8 mm. r(t) begins at a value near that of the distance between the heat source and nearest edge of the sensor, and rapidly approaches the mean sensor distance from the heater. r(t) is, more generally, a function of ρₛₖᵢₙc_{p,skin}t/kₛₖᵢₙ, and the solutions of r(t) for kₛₖᵢₙ=0.35 W m⁻¹ K⁻¹ and ρₛₖᵢₙc_{p,skin}=2.33 J cm⁻³ K⁻¹ are shown in FIGS. 16a-c. While r(t) is a function of thermal properties as well as time, the r(t) values shown in FIG. 16a-c are assumed to be reasonable approximations for all thermal properties encountered on skin in vivo. The error associated with this approximation can be estimated by determining r(t) for one set of thermal property values (the mid-range values of the in vivo data), and equation (S5) is solved for a set of thermal property values different from those used to determine r(t) (high-range values of the in vivo data). Equation (S6) is then solved, where r(t) is fixed and kₛₖᵢₙ and ρₛₖᵢₙc_{p,skin} are varied iteratively to minimize the error between equation (S6) and equation (S5). A typical result from this type of analysis is shown in FIG. 16d, along with the results determined by replacing r(t) with different time independent values (geometric mean, harmonic mean, and r₁). The discrepancy between the results determined by equation (S5) and the approximation using r(t) with equation (S6) are found to be <1%. The still simpler solution using a single, time-independent value in place of r(t) are found to produce errors <5%, if chosen appropriately.

### REFERENCES

1 Webb, R. C. et al. Ultrathin conformal devices for precise and continuous thermal characterization of human skin. Nat Mater 12, 938-944, doi:Doi 10.1038/Nmat3755 (2013).
2 Gustafsson, S. E. Transient plane source techniques for thermal conductivity and thermal diffusivity measurements of solid materials. Review of Scientific Instruments 62, 797-804 (1991).

TABLE 2
Pearson Correlation coefficients for the correlation analyses (FIGS. 4-6).

| | | | | | |
|---|---|---|---|---|---|
| SC | SC | EP | Thermal | Volumetric | |
| Hydration | Thickness | Thickness | Conductivity | Heat Capacity | Diffusivity |

Multivariate
Correlations

| | | | | | | |
|---|---|---|---|---|---|---|
| SC Hydration | 1.0000 | −0.5523 | −0.5479 | 0.5779 | 0.5157 | 0.1376 |
| SC Thickness | −0.5523 | 1.0000 | 0.8957 | −0.7427 | −0.4653 | −0.6446 |
| EP Thickness | −0.5479 | 0.9957 | 1.0000 | −0.7567 | −0.4776 | −0.6465 |
| Thermal Conductivity | 0.5779 | −0.7427 | −0.7567 | 1.0000 | 0.9040 | 0.1774 |
| Volumetric Heat Capacity | 0.5157 | −0.4653 | −0.4775 | 0.9040 | 1.0000 | −0.2551 |
| Diffusivity | 0.1376 | −0.5446 | −0.6455 | 0.1774 | −0.2551 | 1.0000 |

There are 2 missing values. The correlations are estimated by REML method.

Multivariate Location = cheek
Correlations

| | | | | | | |
|---|---|---|---|---|---|---|
| SC Hydration | 1.0000 | 0.0000 | 0.1456 | 0.1504 | 0.2 [symbol] | −0.2964 |
| SC Thickness | 0.0000 | 0.0000 | 0.0000 | 0.0000 | 0.0000 | 0.0000 |
| EP Thickness | 0.1456 | 0.0000 | 1.0000 | −0.0 [symbol] 76 | 0.1772 | −0.2219 |
| Thermal Conductivity | 0.1504 | 0.0000 | 0.0376 | 1.0000 | 0.9418 | −0.7469 |
| Volumetric Heat Capacity | 0.2395 | 0.0000 | 0.1772 | 0.941 [symbol] | 1.0000 | −0.9247 |
| Diffusivity | −0.29 [symbol] 4 | 0.0000 | −0.2219 | −0.7469 | −0.9247 | 1.0000 |

Multivariate Location = d-forearm
Correlations

| | | | | | | |
|---|---|---|---|---|---|---|
| SC Hydration | 1.0000 | 0.0000 | −0.0561 | 0.73 [symbol] | 0.7431 | −0.5789 |
| SC Thickness | 0.0000 | 0.0000 | 0.0000 | 0.0000 | 0.0000 | 0.0000 |
| EP Thickness | −0.0561 | 0.0000 | 1.0000 | 0.0376 | 0.0217 | 0.0334 |
| Thermal Conductivity | 0.73 [symbol] | 0.0000 | 0.0376 | 1.0000 | 0.9746 | −0.7246 |
| Volumetric Heat Capacity | 0.7431 | 0.0000 | 0.0217 | 0.9746 | 1.0000 | −0.8573 |
| Diffusivity | −0.5789 | 0.0000 | 0.0334 | −0.7246 | −0.6573 | 1.0000 |

Multivariate Location = heel
Correlations

| | | | | | | |
|---|---|---|---|---|---|---|
| SC Hydration | 1.0000 | −0. [symbol] 045 | −0.6767 | 0.6433 | 0.3940 | 0.0653 |
| SC Thickness | −0.6045 | 1.0000 | 0.9579 | −0.4 [symbol] 23 | −0.3 [symbol] 62 | 0.0620 |
| EP Thickness | −0.6767 | 0.9579 | 1.0000 | −0.5074 | −0.4049 | 0.0434 |
| Thermal Conductivity | 0. [symbol] 433 | −0.4 [symbol] 23 | −0. [symbol] 074 | 1.0000 | 0. [symbol] 496 | −0.5243 |
| Volumetric Heat Capacity | 0.3 [symbol] 40 | −0. [symbol] 962 | −0.4049 | 0.9496 | 1.0000 | −0.7626 |
| Diffusivity | 0.0653 | 0.0620 | 0.0434 | −0.6243 | −0.762 [symbol] | 1.0000 |

Multivariate Location = palm
Correlations

| | | | | | | |
|---|---|---|---|---|---|---|
| SC Hydration | 1.0000 | −0.5413 | −0.4591 | 0.5784 | 0.4066 | 0.1606 |
| SC Thickness | −0.5413 | 1.0000 | 0.9145 | −0.6861 | −0.4179 | −0.3327 |
| EP Thickness | −0.4691 | 0.9145 | 1.0000 | −0.5601 | −0.3172 | −0.3248 |
| Thermal Conductivity | 0.5784 | −0.6861 | −0.5601 | 1.0000 | 0.9013 | −0.1981 |
| Volumetric Heat Capacity | 0.406 [symbol] | −0.4179 | −0.3172 | 0.9013 | 1.0000 | −0. [symbol] 021 |
| Diffusivity | 0.1606 | −0.3327 | −0.3248 | −0.1981 | −0.6021 | 1.0000 |

Multivariate Location = v-forearm
Correlations

| | | | | | | |
|---|---|---|---|---|---|---|
| SC Hydration | 1.0000 | 1.0000 | −0.060 [symbol] | 0.1420 | 0.1718 | −0.1683 |
| SC Thickness | 1.0000 | 1.0000 | −0.060 [symbol] | 0.1426 | −0.1718 | −0.1683 |
| EP Thickness | −0.0608 | −0.0608 | 1.0000 | −0.4181 | −0.3645 | 0.2396 |
| Thermal Conductivity | 0.1426 | 0.1426 | −0.4181 | 1.0000 | 0. [symbol] 587 | −0.6740 |
| Volumetric Heat Capacity | 0.1718 | 0.1718 | −0.3845 | 0.9587 | 1.0000 | −0.8546 |
| Diffusivity | −0.1683 | −0.1683 | 0.2396 | −0.6740 | −0.8546 | 1.0000 |

There are 2 missing values. The correlations are estimated by REML method.

Multivariate Location = wrist
Correlations

| | | | | | | |
|---|---|---|---|---|---|---|
| SC Hydration | 1.0000 | 0.0000 | −0.2143 | 0.4363 | 0.4167 | −0.2230 |
| SC Thickness | 0.0000 | 0.0000 | 0.0000 | 0.0000 | 0.0000 | 0.0000 |
| EP Thickness | −0.2143 | 0.0000 | 1.0000 | −0.1626 | −0.0179 | −0.3725 |
| Thermal Conductivity | 0.4363 | 0.0000 | −0.1626 | 1.0000 | 0.9659 | −0.4 [symbol] |
| Volumetric Heat Capacity | 0.4167 | 0.0600 | −0.0179 | 0.9659 | 1.0000 | −0.6334 |
| Diffusivity | −0.2230 | 0.0000 | −0.3725 | −0.4863 | −0.6934 | 1.0000 |

[symbol]

indicates data missing or illegible when filed

### Example 2: Clinical Studies of Thermal Transport Characteristics of Human Skin Measured In Vivo Using Ultrathin Conformal Arrays of Thermal Sensors and Actuators

Study Details:

Patients: 10 women, aged 18-30, and 10 women, aged 50-65.

Stimulus:

Glycerin (glycerine in water solution) of varying compositions from 0%-30% on randomized locations on patients' right volar forearm. Serves as humectant, which is a diffusion barrier to prevent transepidermal water loss (TEWL). [1]

Occlusive Patch:

Physical barrier preventing water from escaping from Stratum Corneum.

Measurements:

Transepidermal Water Loss (TEWL) (Commercial).

Corneometer (Commercial).

Epidermal thermal transport measurement.

Epidermal impedance measurement.

Time Points Legend:

T0 BPA=Before stimulus is applied (baseline)

TI mm=15 mins after stimulus is applied

T30=30 mins after stimulus is applied

T60=60 mins after stimulus is applied

T330=330 mins after stimulus is applied

Tend=After solution has been wiped off (baseline).

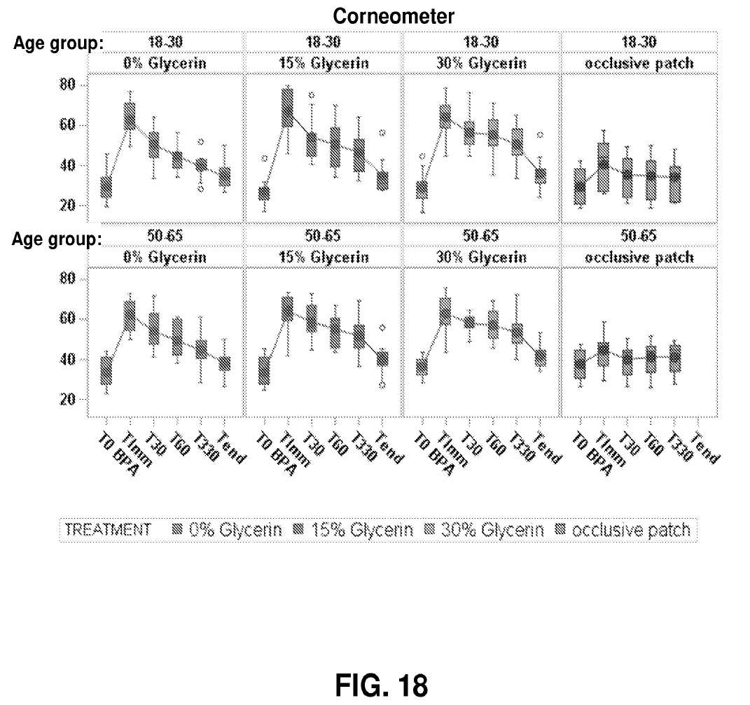

FIG. 18: Corneometer (CM 825®, Courage+Khazaka electronic GmbH) measurement (capacitance-based measurement) at locations where stimulus is applied at defined time points. Shows strong peak at TI time point for both age groups, probably corresponding to initial water evaporation from glycerine solution. Measurements reach baseline at Tend time point. Occlusive patch has much smaller effect, as expected. Measurement serves as main validation of experimental epidermal sensor being tested.

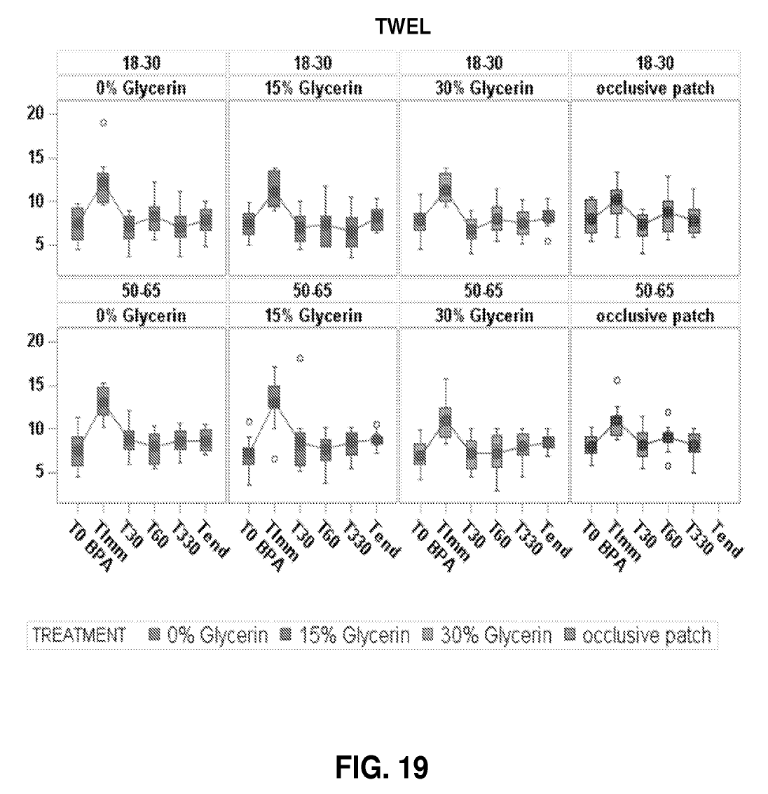

FIG. 19: Transepidermal Water Loss (TEWL) (Vapometer®, Delfin Technologies) measurements, for both age groups using defined time points and stimuli, as measured from stratum corneum. Data show a strong peak at TI, immediately after stimulus is applied, corresponding to loss in water in solution, consistent for both age groups. Occlusive patch has much smaller effect for both age groups, as expected.

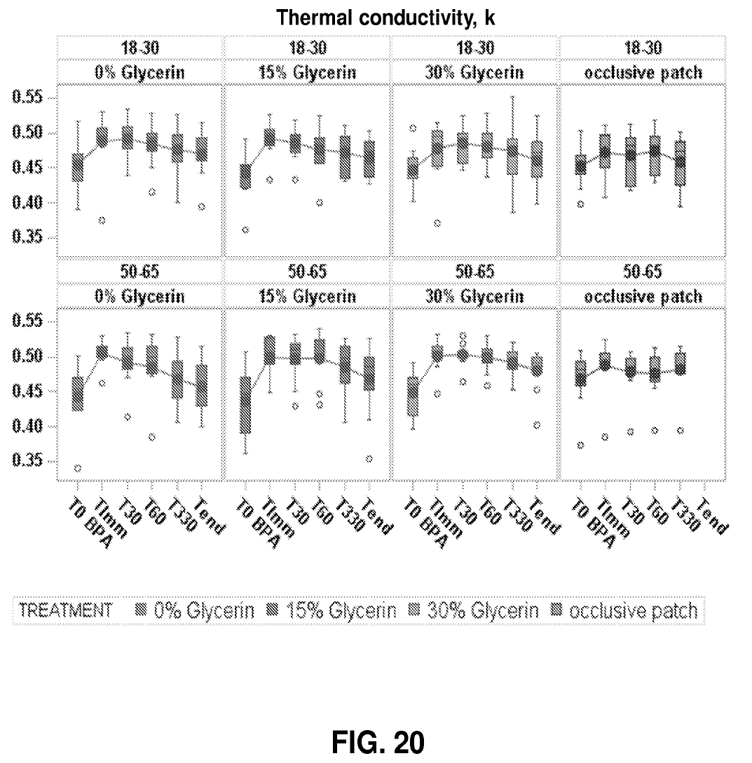

FIG. 20: Skin thermal conductivity (kₛₖᵢₙ) measurements using an epidermal electronic system for both age groups using defined time points and stimuli. Shows a clear increase in thermal conductivity with hydration, as expected.

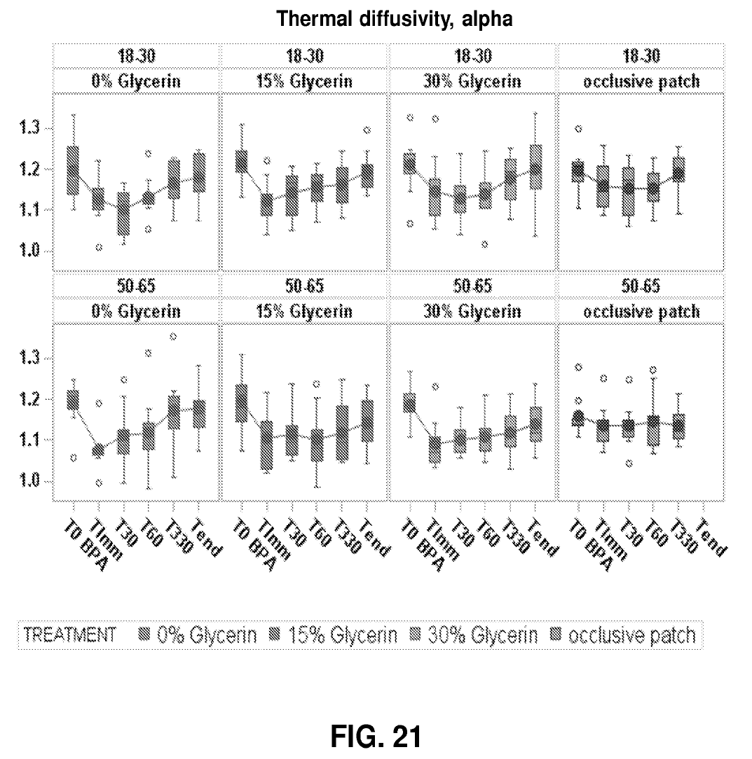

FIG. 21: Thermal diffusivity

$$( {α}_{skin} = \frac{{k}_{skin}}{{ρ}_{skin} {c}_{p , skin}} )$$

measurements using an epidermal electronic system for both age groups using defined time points and stimuli. Shows a decrease with increased hydration, due to increased specific heat capacity of skin with hydration.

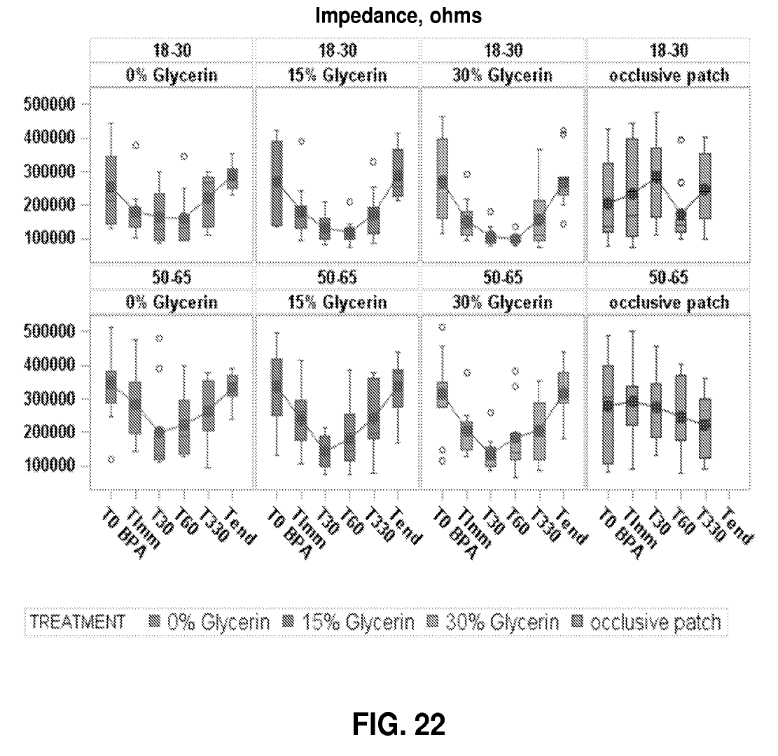

FIG. 22: Impedance magnitude measurements

$$( {z}_{skin} = \frac{V}{I} )$$

using an epidermal electronic system for both age groups using defined time points and stimuli. Shows a strong decrease with increased hydration, as expected, suggesting peak hydration levels at either the T30 or T60 time points for both age groups.

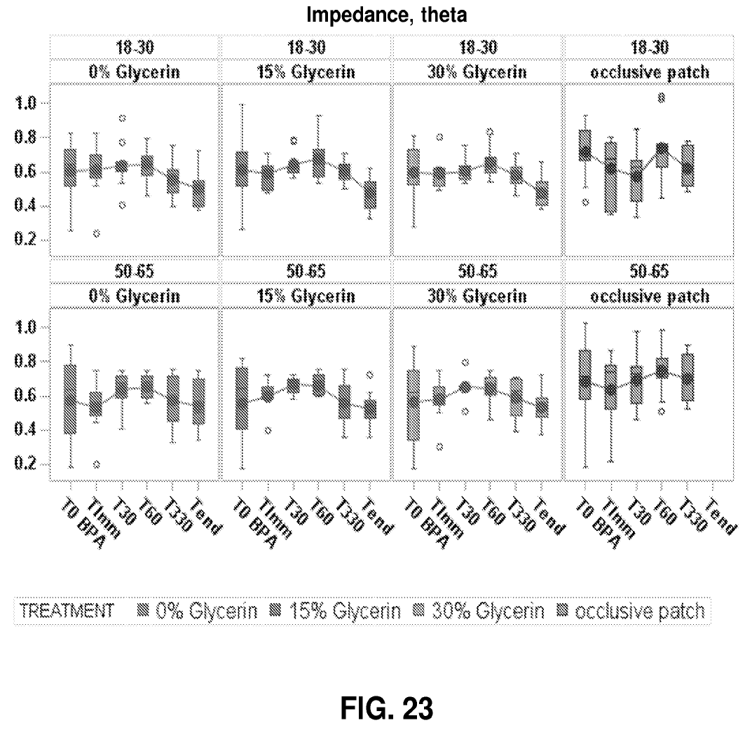

FIG. 23: Impedance phase angle

$$( θ = {tan}^{- 1} ( \frac{V}{I} ) ) )$$

using an epidermal electronic system for both age groups using defined time points and stimuli. Can also be used as an indicator of hydration level.

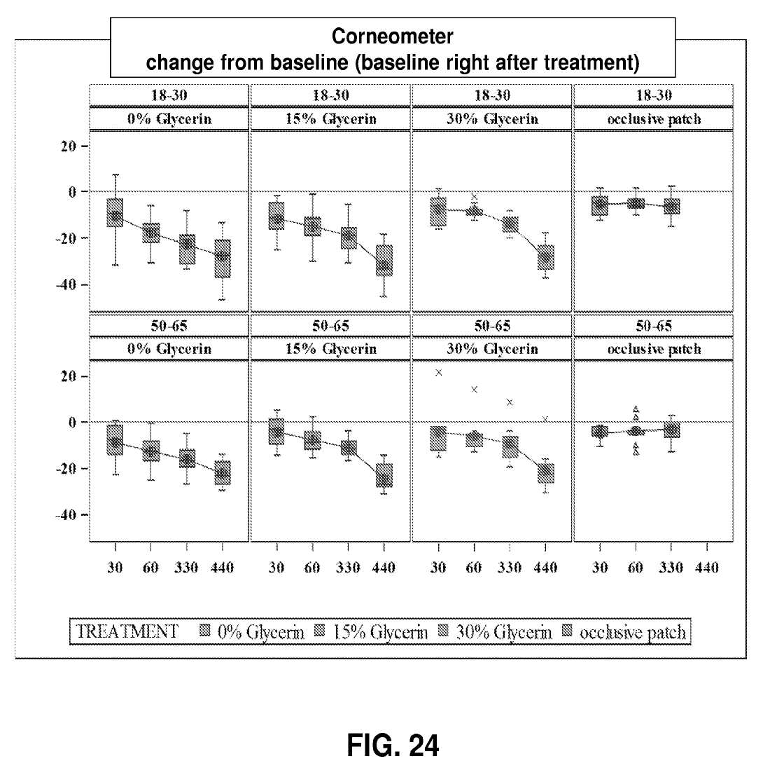

FIG. 24: FIG. 18 replotted with TI (initial time point after stimulus is applied) as the baseline. Shows change in measured value after initial application of stimulus.

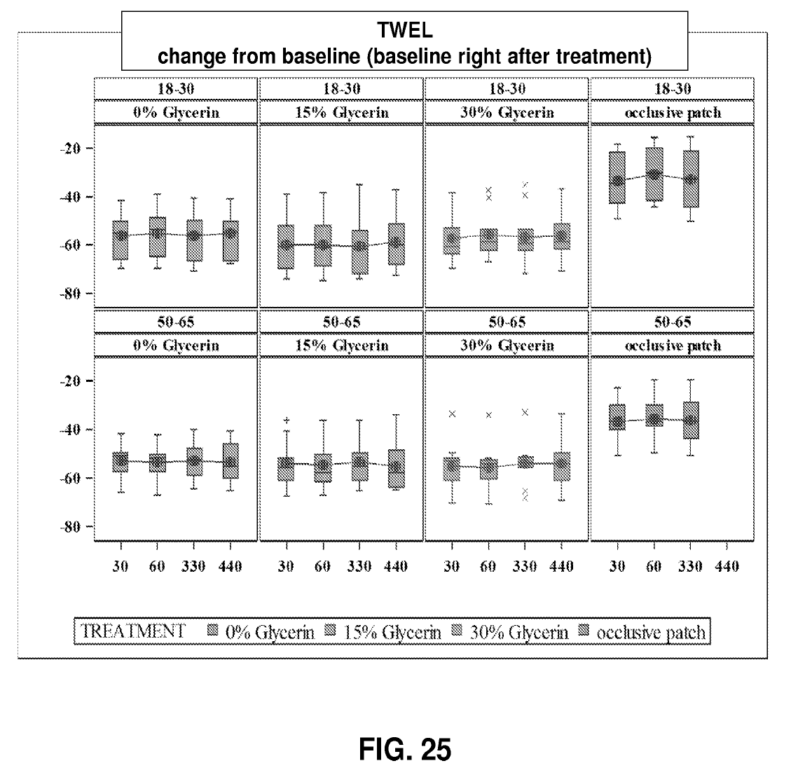

FIG. 25: FIG. 19 replotted with TI (initial time point after stimulus is applied) as the baseline. Shows change in measured value after initial application of stimulus.

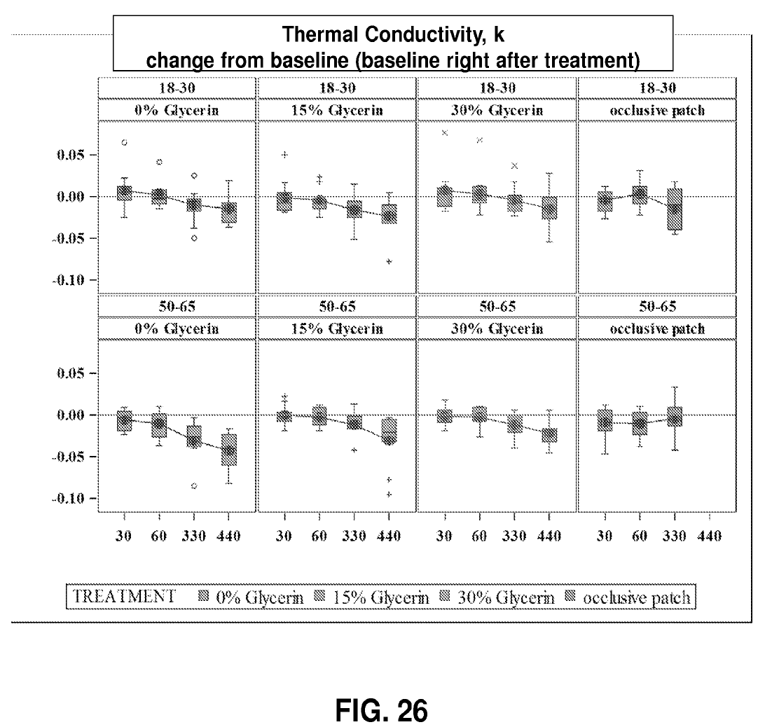

FIG. 26: FIG. 20 replotted with TI (initial time point after stimulus is applied) as the baseline. Shows change in measured value after initial application of stimulus.

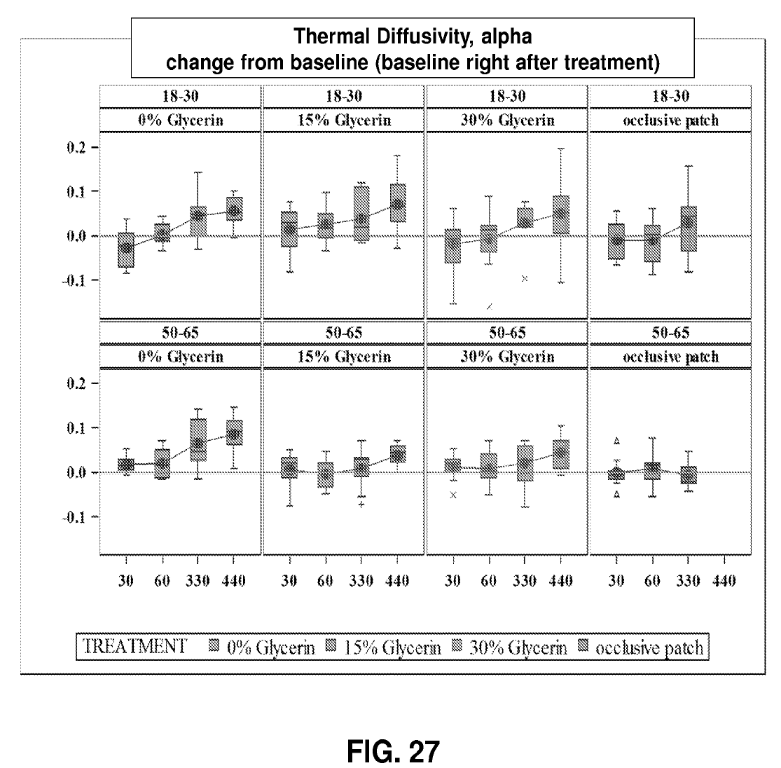

FIG. 27: FIG. 21 replotted with TI (initial time point after stimulus is applied) as the baseline. Shows change in measured value after initial application of stimulus.

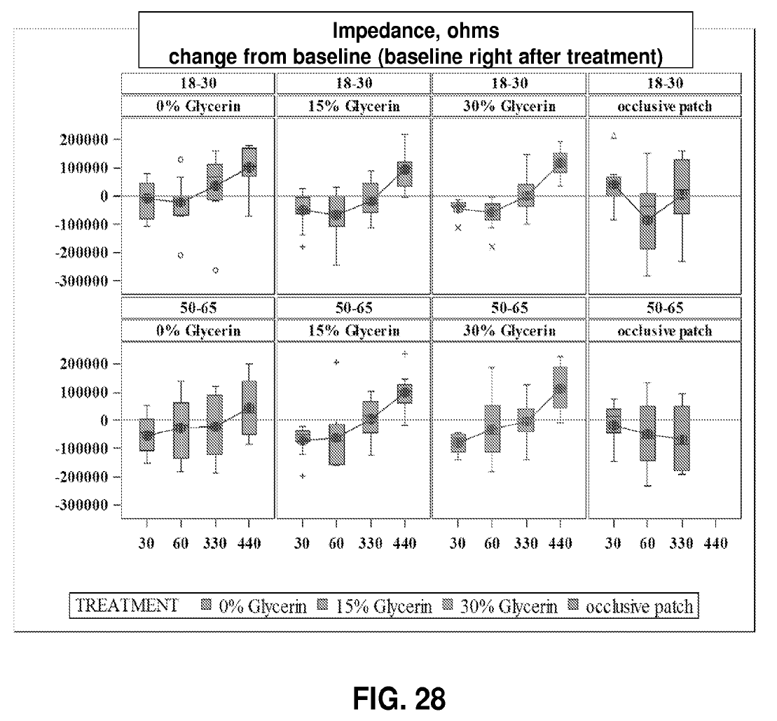

FIG. 28: FIG. 22 replotted with TI (initial time point after stimulus is applied) as the baseline. Shows change in measured value after initial application of stimulus.

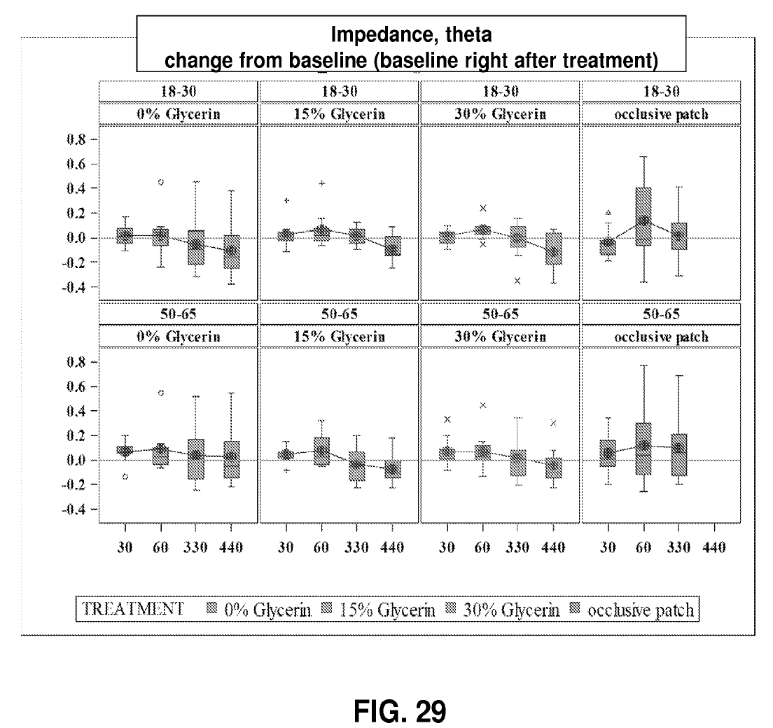

FIG. 29: FIG. 23 replotted with TI (initial time point after stimulus is applied) as the baseline. Shows change in measured value after initial application of stimulus.

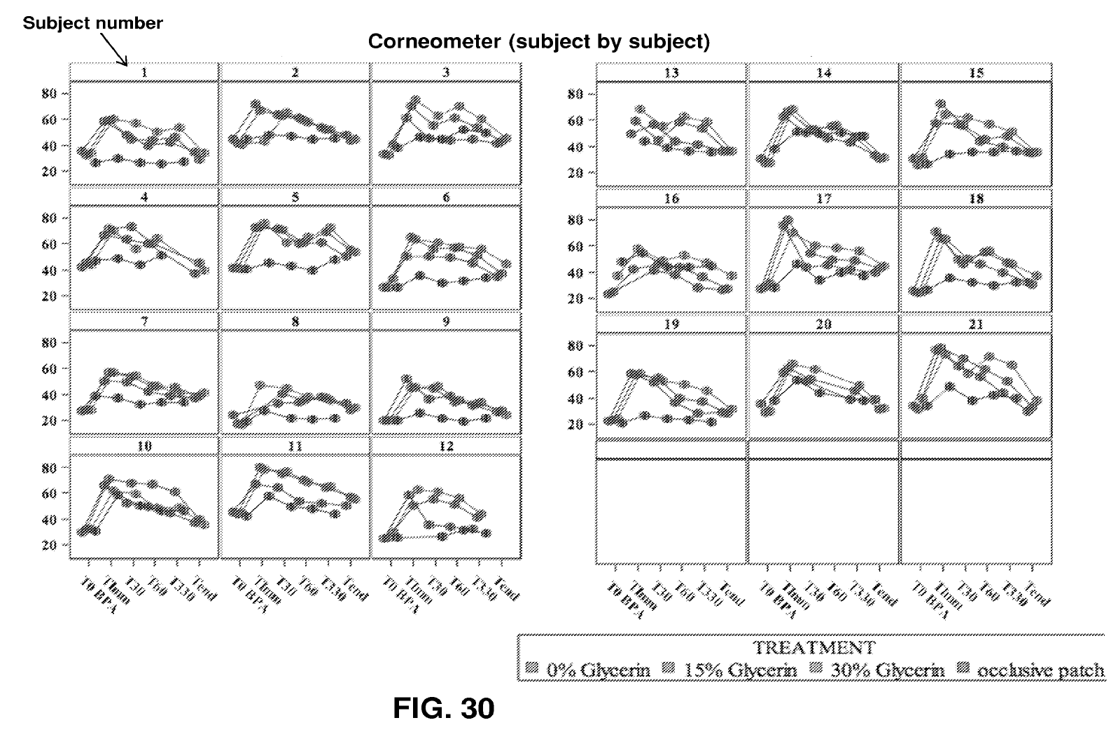

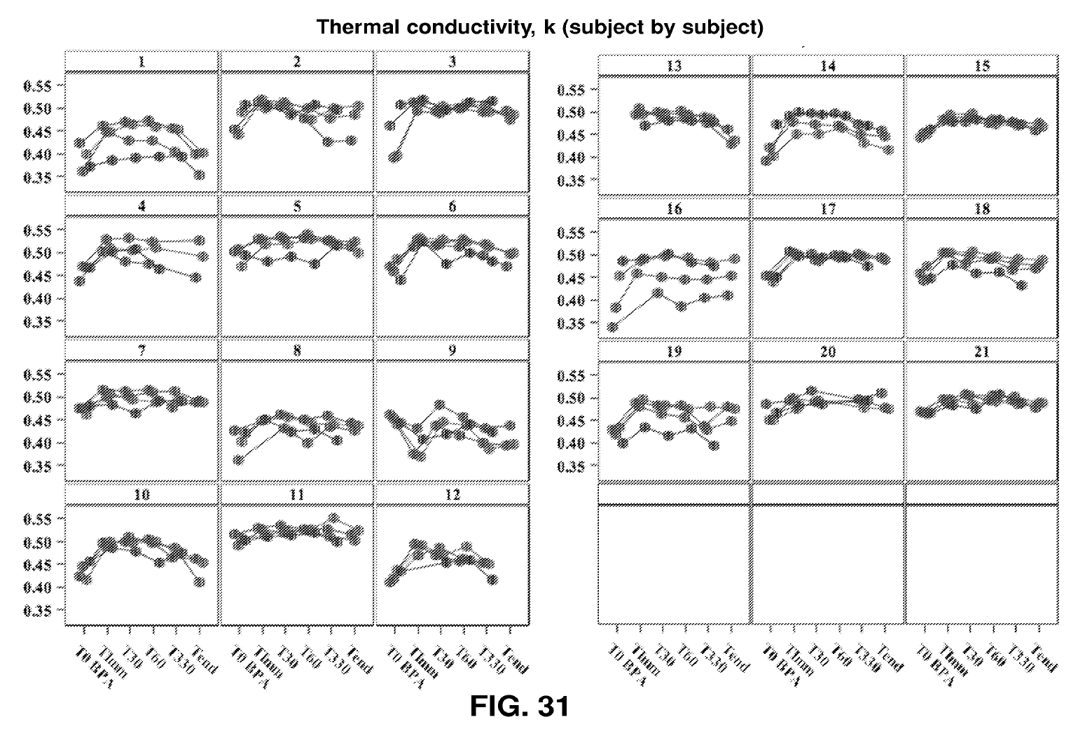

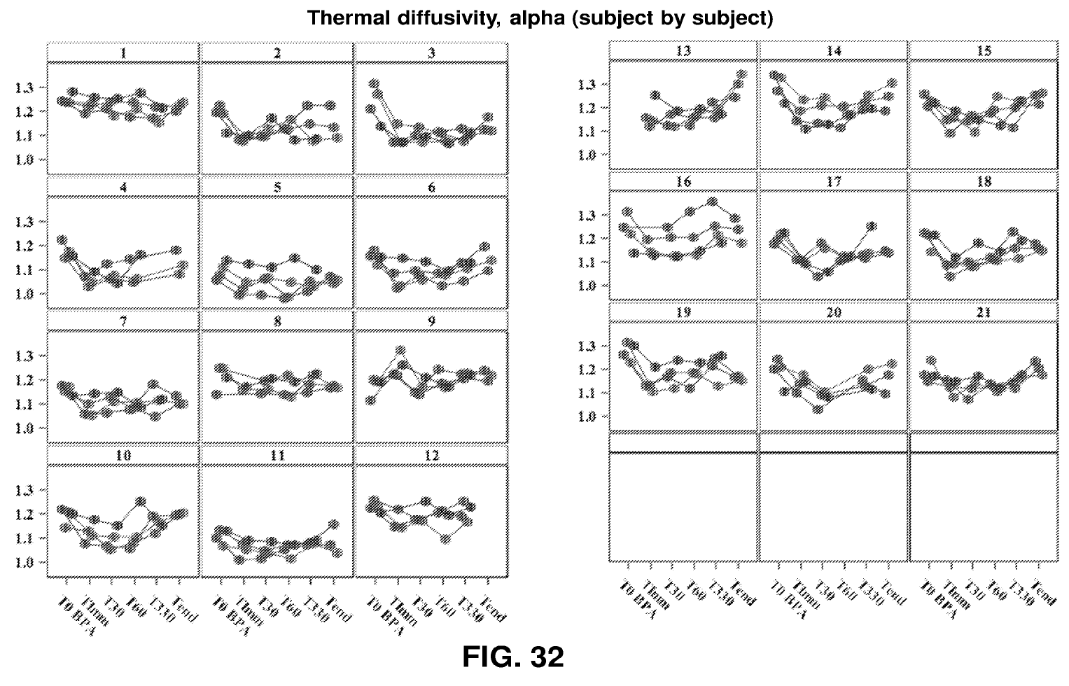

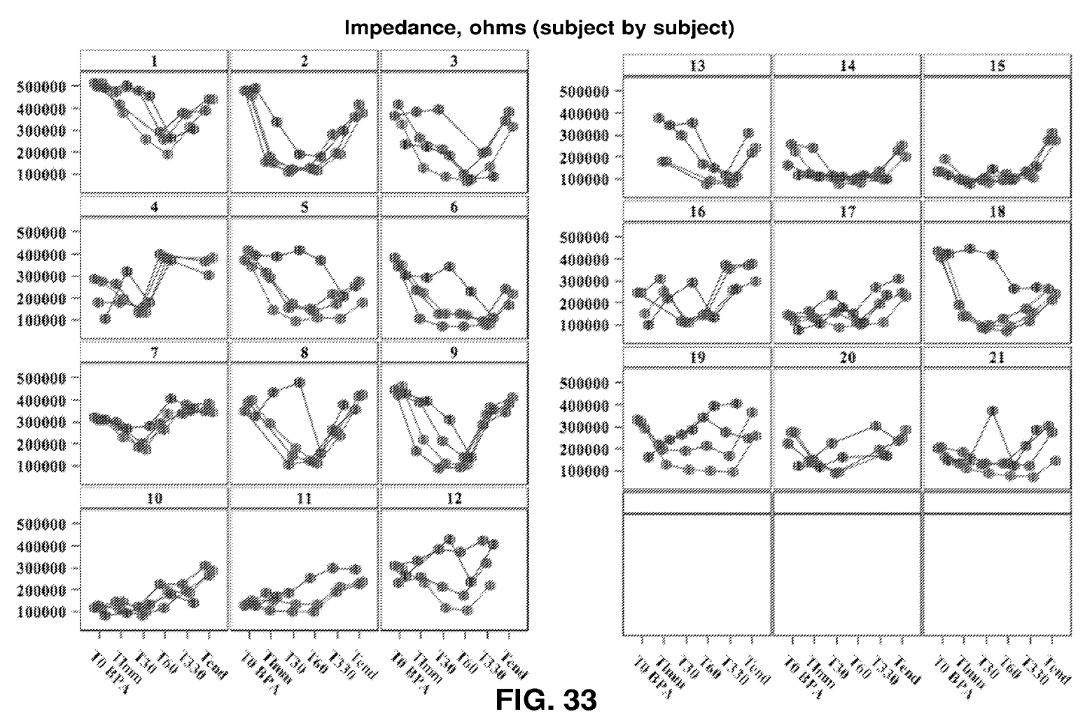

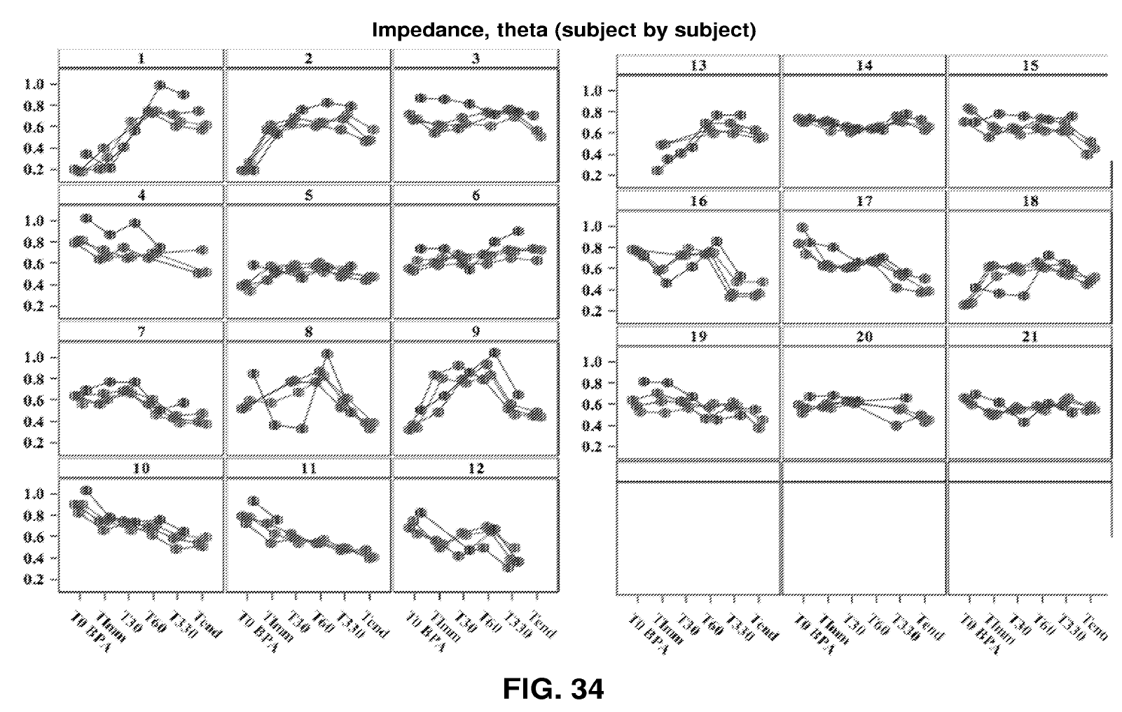

FIGS. 30-34: Raw data for every patient for stimuli and measurement modes shown in FIGS. 18-29.

### REFERENCES

1 Batt, M. D. and E. Fairhurst, HYDRATION OF THE STRATUM-CORNEUM. International Journal of Cosmetic Science, 1986. 8(6): p. 253-264.
2 Webb, R. C., et al., Thermal transport characteristics of human skin measured in vivo using ultrathin conformal arrays of thermal sensors and actuators. PLoS One, 2015. 10(2): p. e0118131.
3 Huang, X., et al., Epidermal impedance sensing sheets for precision hydration assessment and spatial mapping. Biomedical Engineering, IEEE Transactions on, 2013. 60(10): p. 2848-2857.

### Example 3: Impedance-Based Hydration Measurements

Measuring Principle:

The outermost skin layer, the stratum corneum, is typically between 15 μm-40 μm thick, and consists of mainly keratinized cells. Beneath the stratum corneum are the dermis and the epidermis, (roughly 100 μm and around 400 μm thick, respectively). The stratum corneum acts as a highly resistive layer, while the underlying layers, consisting of mainly granular cells, have a strong capacitive component to their impedance [1]. The application of an AC current to skin-mounted electrodes can be used to measure impedance, which corresponds strongly to hydration levels in the stratum corneum [3]. This forms the basis of traditional capacitive or impedance based techniques used to measure skin hydration levels [4]. Traditionally, concentric circular electrodes are employed, and the geometry and spacing of the electrodes strongly influences the measurement depth, with measurement depth approximated as roughly half the spacing between the two electrodes [5]. An analytical equation for the impedance of a concentric coplanar capacitor on multilayered skin has been developed by Cheng et al. [6], and is given by:

$$Z = \frac{2}{π ( {σ}_{SC} + j ω {\overline{ɛ}}_{SC} )} {\int }_{0}^{∞} {κ}^{2} ( ξ ) \frac{( ω {\overline{ɛ}}_{D} - j {σ}_{D} ) tanh ( ξ {h}_{SC} ) tanh ( ξ {h}_{D} ) + ( ω {\hat{ɛ}}_{SC} - j {σ}_{SC} )}{( ω {\overline{ɛ}}_{D} - j {σ}_{D} ) tanh ( ξ {h}_{D} ) + ( ω {\hat{ɛ}}_{SC} - j {σ}_{SC} ) tanh ( ξ {h}_{SC} )} d ξ .$$

Where σ_{sc} is the conductivity of the stratum corneum, ω_{sc} is the measurement frequency, ∈ is the dielectric constant of the stratum corneum, and ξ_{sc} and κ_{sc} are parameters that account for the device geometry and spacing.

Electrode Sizes:

The inner electrode can have a radius from 50 μm to 200 μm, while the outer electrode can have a typical inner radius between 100 and 300 μm. Spacings too small risk short circuiting the electrode, while spacings too large will create extremely large measurement depths, and the amount of useful information will be limited.

Frequency Dependence:

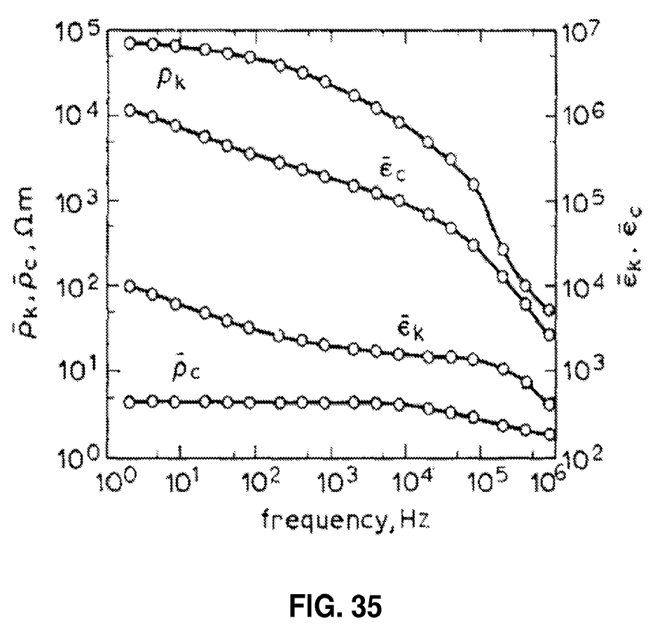

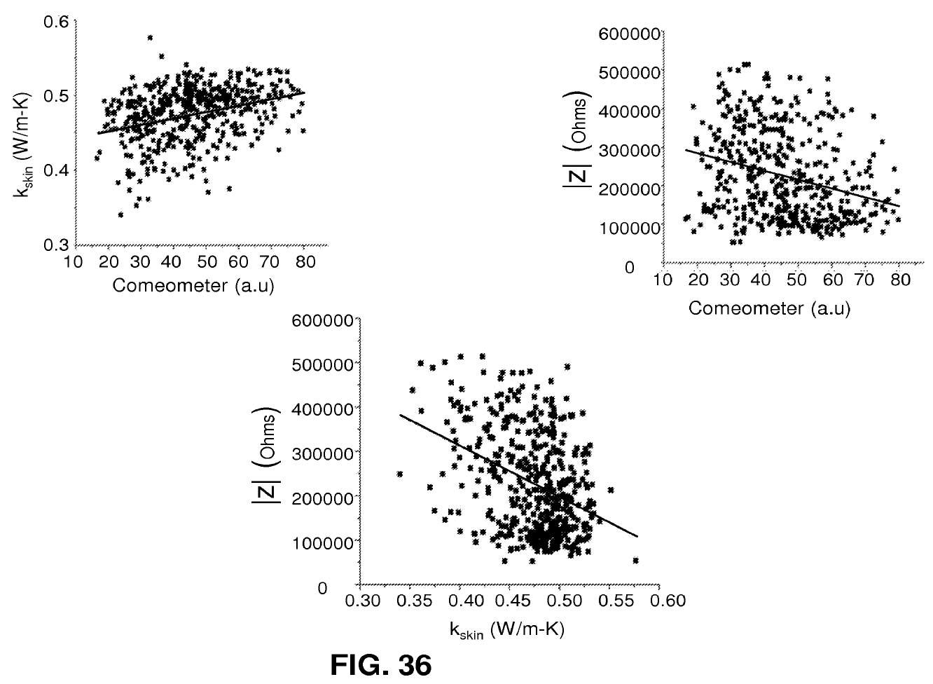

The frequency range for such measurements can vary by 5 orders of magnitude from 10 Hz to 1 MHz. Due to dispersion effects, the resistivity of the stratum corneum diminishes strongly over such a frequency range, and converges with the resistivity of the underlying viable skin layers. The dielectric constant of the stratum corneum also diminishes over this frequency range, and converges to the value of the dielectric constant of the underlying viable skin layers, as illustrated in FIG. 35 [1]. In general, the resistivity and the dielectric constant of both skin layers converge at high frequencies to values much closer to those of the viable skin layers, with the result that high frequency measurements read much stronger contributions from the underlying skin layers [7, 8].

Advantages of Multimodal Impedance/Thermal Measurement:

The fundamental advantage of multimodal impedance and thermal measurement is the unprecedented ability to make simultaneous, independent measurements on the same patient, on the same body location and essentially at the same time.

Error and uncertainty analysis is facilitated by comparing the two measurements with each other. This is especially relevant given the high level of uncertainty inherent in traditional commercial techniques.

Further, the mechanics of the device are the same for both measurement modes, and identical contact pressure, adhesion and skin conditions can be assumed for both techniques.

Both techniques provide for the control of measurement depth: measurement time in the case of the thermal analysis and measurement frequency and electrode spacing in the case of impedance measurements. The ability to control measurement depth allows for the determination and validation of hydration permeation, skin diffusivity and the effects of humectants, emollients and other topical compounds, with applications in cosmetology, dermatology and toxicology.

### REFERENCES

1 Yamamoto, T. and Y. Yamamoto, Electrical properties of the epidermal stratum corneum. Medical and Biological Engineering, 1976. 14(2): p. 151-158.
2 Webb, R. C., et al., Thermal transport characteristics of human skin measured in vivo using ultrathin conformal arrays of thermal sensors and actuators. PLoS One, 2015. 10(2): p. e0118131.
3 Batt, M. D. and E. Fairhurst, HYDRATION OF THE STRATUM-CORNEUM. International Journal of Cosmetic Science, 1986. 8(6): p. 253-264.
4 Alanen, E., et al., Measurement of hydration in the stratum corneum with the MoistureMeter and comparison with the Corneometer. Skin Research and Technology, 2004. 10(1): p. 32-37.
5 Åberg, P., et al., Skin cancer identification using multifrequency electrical impedance-a potential screening tool. Biomedical Engineering, IEEE Transactions on, 2004. 51(12): p. 2097-2102.
6 Cheng, H., et al., Analysis of a concentric coplanar capacitor for epidermal hydration sensing. Sensors and Actuators A: Physical, 2013. 203: p. 149-153.
7 Martinsen, O. G. and S. Grimnes, Bioimpedance and bioelectricity basics. 2011: Academic press.
8 Martinsen, Ø. G., S. Grimnes, and E. Haug, Measuring depth depends on frequency in electrical skin impedance measurements. Skin Research and Technology, 1999. 5(3): p. 179-181.

### Statements Regarding Incorporation by Reference and Variations

All references throughout this application, for example patent documents including issued or granted patents or equivalents; patent application publications; and non-patent literature documents or other source material are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods and steps set forth in the present description. As will be obvious to one of skill in the art, methods and devices useful for the present embodiments can include a large number of optional composition and processing elements and steps.

When a group of substituents is disclosed herein, it is understood that all individual members of that group and all subgroups, including any isomers, enantiomers, and diastereomers of the group members, are disclosed separately. When a Markush group or other grouping is used herein, all individual members of the group and all combinations and subcombinations possible of the group are intended to be individually included in the disclosure. When a compound is described herein such that a particular isomer, enantiomer or diastereomer of the compound is not specified, for example, in a formula or in a chemical name, that description is intended to include each isomer and enantiomer of the compound described individually or in any combination. Additionally, unless otherwise specified, all isotopic variants of compounds disclosed herein are intended to be encompassed by the disclosure. For example, it will be understood that any one or more hydrogens in a molecule disclosed can be replaced with deuterium or tritium. Isotopic variants of a molecule are generally useful as standards in assays for the molecule and in chemical and biological research related to the molecule or its use. Methods for making such isotopic variants are known in the art. Specific names of compounds are intended to be exemplary, as it is known that one of ordinary skill in the art can name the same compounds differently.

Every formulation or combination of components described or exemplified herein can be used to practice the invention, unless otherwise stated.

Whenever a range is given in the specification, for example, a number range, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art. For example, when compositions of matter are claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements and/or limitation or limitations, which are not specifically disclosed herein.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

It must be noted that as used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art, and so forth. As well, the terms “a” (or “an”), “one or more” and “at least one” can be used interchangeably herein. It is also to be noted that the terms “comprising”, “including”, and “having” can be used interchangeably. The expression “of any of claims XX-YY” (wherein XX and YY refer to claim numbers) is intended to provide a multiple dependent claim in the alternative form, and in some embodiments is interchangeable with the expression “as in any one of claims XX-YY.”

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are described.

In certain embodiments, the invention encompasses administering a medical device of the invention to a patient or subject. A “patient” or “subject”, used equivalently herein, refers to an animal. In particular, an animal refers to a mammal, preferably a human. The subject can either: (1) have a condition able to be monitored, diagnosed, prevented and/or treated by administration of a medical device of the invention; or (2) is susceptible to a condition that is able to be monitored, diagnosed, prevented and/or treated by administering a medical device of the invention.

When used herein, the terms “diagnosis”, “diagnostic” and other root word derivatives are as understood in the art and are further intended to include a general monitoring, characterizing and/or identifying a state of health or disease. The term is meant to encompass the concept of prognosis. For example, the diagnosis of cancer can include an initial determination and/or one or more subsequent assessments regardless of the outcome of a previous finding. The term does not necessarily imply a defined level of certainty regarding the prediction of a particular status or outcome.

As defined herein, “administering” means that a device of the invention is provided on epidermal tissue of a patient or subject. The invention includes methods for applying or adhering a device in vivo to the epidermis of a patient in need of treatment, for example to a patient undergoing treatment for a diagnosed diseased state. Administering can be carried out by a range of techniques known in the art.

1. A method of sensing an epidermal tissue of a subject, the method comprising:
thermally actuating an epidermal tissue region with one or more thermal elements by delivering a heating power selected from the range of 0.0001 mJ s⁻¹ to 1000 mJ s⁻¹ for a period selected from the range of 10 ms to 1000 s;
detecting one or more temperatures of said epidermal tissue proximate to said tissue region with said one or more thermal elements; and
generating a depth profile thermal measurement.

2. The method of claim 1, wherein said step of generating comprises analyzing said one or more temperatures of said epidermal tissue to provide said depth profile thermal measurement, wherein said depth profile thermal measurement is thermal conductivity, thermal diffusivity or heat capacity as a function of three-dimensional tissue location.

3. (canceled)

4. The method of claim 1, wherein said step of generating said depth profile thermal measurement comprises varying said thermal actuation by varying thermal heating power or duration to provide a multifocal response.

5-6. (canceled)

7. The method of claim 1, wherein said depth profile thermal measurement is used to determine a three-dimensional hydration profile of said tissue or a three dimensional circulation profile of tissue.

8-10. (canceled)

11. The method of claim 1 further comprising electrically actuating said epidermal tissue region with a first electrode and obtaining an electrical signal from a second epidermal tissue region with a second electrode, wherein said first electrode and said second electrode are separated by a distance selected from the range of 50 μm to 10 mm, and wherein said depth profile extends from a surface of said epidermal tissue to a depth equal to half the separation distance between the first electrode and the second electrode, and wherein said first electrode delivers alternating current having a frequency of 1 kHz to 100 KHz.

12-14. (canceled)

15. The method of claim 1, wherein said one or more thermal elements are provided in conformal contact with said tissue, thereby providing said one or more thermal elements in thermal contact with the epidermal tissue, and wherein said one or more thermal elements are thermal actuators and sensors.

16. The method of claim 1, wherein detecting one or more temperatures of said epidermal tissue proximate to said tissue region comprises measuring a distribution of said temperatures of said surface of said epidermal tissue in response to said thermally actuating step or comprises spatio temporally mapping the temperatures of said surface of said epidermal tissue in response to said thermally actuating step.

17-18. (canceled)

19. The method of claim 1, wherein said step of delivering a heating power comprises delivering said heating power selected from the range of 1 mW mm⁻² to 10 mW mm⁻²; or comprises delivering said heating power for a duration of 2 seconds to 8 hours; or comprises delivering said heating power over an area of said tissue selected from the range of 0.0001 mm² to 1 cm²;

20-21. (canceled)

22. The method of claim 1, where said thermally actuating comprises applying a continuous heating power to said epidermal tissue or comprises applying a pulsed heating power to said epidermal tissue, wherein the pulsed power has a frequency between 0.001 Hz and 10 Hz with a duty cycle between 0.001% and 100% duty cycle.

23-24. (canceled)

25. The method of claim 1, wherein said step of thermally actuating and said step of detecting temperature are carried out sequentially, wherein each of said one or more thermal elements actuates then detects; or wherein said step of thermally actuating is carried out by a first portion of said one or more thermal elements and wherein said step of detecting temperature is carried out by a second portion of said one or more thermal elements, wherein said steps occur sequentially or wherein said steps occur simultaneously; and
also wherein said step of detecting one or more temperatures occurs at a frequency selected from the range of 0.0001 s⁻¹ to 1000 s⁻¹; or wherein said step of detecting one or more temperatures provides a temperature measurement characterized by a temporal resolution selected from 1 ms to 1000 s; or wherein said step of detecting one or more temperatures provides a temperature measurement characterized by a spatial resolution selected from 0.01 mm to 1 cm; or wherein said step of detecting one or more temperatures provides a temperature measurement characterized by a thermal resolution selected from 0.001° C. to 10° C.

26-32. (canceled)

33. The method of claim 1, wherein said step of thermally actuating increases the temperatures of said epidermal tissue by less than 20° C. and wherein said step of detecting one or more temperatures corresponds to tissue having temperatures selected from the range of 0° C. to 50° C.

34. (canceled)

35. The method of claim 1 further comprising a step of determining one or more thermal transport properties of said epidermal tissue using one or more temperatures of said epidermal tissue, wherein said thermal transport property is thermal conductivity, thermal diffusivity or heat capacity per unit volume.

36. (canceled)

37. The method of claim 36, wherein said one or more thermal transport properties are determined using one or more of the relationships:
$$T = {T}_{∞} + {A}_{1} \frac{Q}{2 π {A}_{2} {k}_{skin}} erfc ( \frac{{A}_{2} \sqrt{{ρ}_{skin} {c}_{p , skin}}}{\sqrt{4 {k}_{skin} t}} ) \qquad ( 1 )$$
where T_{∞} is the temperature before heating, Q is the heating power, kₛₖᵢₙ is the thermal conductivity of the skin, ρₛₖᵢₙc_{p,skin} is the volumetric heat capacity of skin, t is time, erfc is the complementary error function, A₂ represents the effective distance from the thermal actuator, and A₁ is a parameter that accounts for details associated with the multilayered geometry of the device;
$$T = {T}_{∞} + {A}_{1} \frac{{\int }_{{r}_{1}}^{{r}_{2}} \{ \frac{Q}{2 π {rk}_{skin}} erfc ( \frac{r \sqrt{{ρ}_{skin} {c}_{p , skin}}}{\sqrt{4 {k}_{skin} t}} ) dr \}}{{r}_{2} - {r}_{1}} \qquad ( 2 )$$
where T is the temperature at a sensor some distance away from the actuator, T_{∞} is the temperature before heating, Q is the heating power, kₛₖᵢₙ is the thermal conductivity of the skin, ρₛₖᵢₙc_{p,skin} is the volumetric heat capacity of skin, t is time, erfc is the complementary error function, r₁ is distance between the actuator and near edge of the sensor to the actuator, r₂ is distance between the actuator and near edge of the sensor to the actuator, and A₁ is a parameter that accounts for details associated with the multilayered geometry of the device; and
$$T = {T}_{∞} + {A}_{1} \frac{Q}{2 π r ( t ) {k}_{skin}} erfc ( \frac{r ( t ) \sqrt{{ρ}_{skin} {c}_{p , skin}}}{\sqrt{4 {k}_{skin} t}} ) \qquad ( 3 )$$
where T_{∞} is the temperature before heating, Q is the heating power, kₛₖᵢₙ is the thermal conductivity of the skin, ρₛₖᵢₙc_{p,skin} is the volumetric heat capacity of skin, t is time, erfc is the complementary error function, A₁ is a parameter that accounts for details associated with the multilayered geometry of the device, and r(t) represents the effective distance of the thermal sensor from the thermal actuator.

38. The method of claim 35, further comprising determining one or more tissue parameters using said thermal transport property, wherein said one or more tissue parameters is hydration state, stratum corneum thickness, epidermis thickness and vasculature structure, and wherein when the tissue parameter is hydration state, said hydration state has independent linear relationships with thermal conductivity and thermal diffusivity

39-41. (canceled)

42. The method of claim 1 further comprising determining the presence, absence or stage of a disease condition for said epidermal tissue of said subject.

43. (canceled)

44. The method of claim 1 further comprising steps of applying a dermatological compound to said surface of said epidermal tissue of said subject and analyzing said tissue temperatures to determine a clinical effectiveness or safety of a dermatological compounds on said tissue, wherein said tissue is follicular tissue or a palmar tissue which corresponds to the face, torso, arms, legs, back, hands or foot of said subject.

45-46. (canceled)

47. The method of claim 1 further comprising contacting a device comprising said one or more thermal elements with a receiving surface of said epidermal tissue, wherein contact results in conformal contact with said receiving surface, thereby providing said one or more thermal elements in thermal contact with the epidermal tissue, wherein said step of contacting provides a contact area of said device with said epidermal tissue surface having an area selected from the range of 0.0001 mm² to 1 cm².

48-67. (canceled)

68. A device for sensing epidermal tissue of a subject, comprising:
a stretchable or flexible substrate;
one or more thermal elements supported by said flexible or stretchable substrate, said one or more thermal elements for:
thermally actuating said tissue with said one or more thermal elements by delivering a heating power selected from the range of 0.0001 mJ s⁻¹ and 1000 mJ s⁻¹ for a period selected from the range of 10 ms to 1000 s;
detecting one or more temperatures of said epidermal tissue proximate to said tissue region with said one or more thermal elements; and
generating a depth profile thermal measurement;
wherein said flexible or stretchable substrate and said one or more thermal elements provide a net bending stiffness low enough such that the device is capable of establishing conformal contact with a receiving surface of the epidermal tissue.

69. The device of claim 68, further comprising a processor in communication with one or more of said thermal elements for receiving and analyzing said temperature measurements to determine one or more thermal transport properties or tissue properties, and wherein said thermal elements of said device are at least partially encapsulated in said substrate or one or more encapsulation layers, wherein said thermal elements comprise stretchable or flexible structures, and wherein said thermal elements comprise thin film structures, or wherein said thermal elements comprise filamentary metal structures.

70-73. (canceled)

74. The device of claim 68, wherein the device has a modulus within a factor of 1000 of a modulus of the epidermal tissue at the interface with the device, or wherein the device has an average modulus less than or equal to 100 MPa; or wherein the device has an average thickness less than or equal to 3000 microns; wherein the device has a net bending stiffness less than or equal to 1 mN m; or wherein the device exhibits a stretchability without failure of greater than 5%.

75-78. (canceled)

79. The device of claim 68, further comprising a first electrode for electrically actuating said epidermal tissue region and a second electrode for obtaining an electrical signal from a second epidermal tissue region, wherein said first electrode and said second electrode are separated by a distance selected from the range of 50 μm to 10 mm, and wherein said first and second electrodes are in direct contact with said epidermal tissue.

80-81. (canceled)

82. The device of claim 68, wherein the device further comprises one or more amplifiers, strain gauges, temperature sensors, wireless power coils, solar cells, inductive coils, high frequency inductors, high frequency capacitors, high frequency oscillators, high frequency antennae, multiplex circuits, electrocardiography sensors, electromyography sensors, electroencephalography sensors, electrophysiological sensors, thermistors, transistors, diodes, resistors, capacitive sensors, light emitting diodes, superstrate, embedding layers, encapsulating layers, planarizing layers or any combinations of these.

83. A method for determining a thermal transport property of a epidermal tissue, the method comprising:
thermally actuating said epidermal tissue with one or more thermal actuators of a device in conformal contact with said epidermal tissue;
measuring temperature of said epidermal tissue with one or more thermal sensors of said device;
determining an effective distance of said one or more thermal sensors from said one or more thermal actuators; and
utilizing said effective distance to determine said thermal transport property of said epidermal tissue.

84. (canceled)

85. The method of claim 83, wherein said effective distance of said one or more thermal sensors from said one or more thermal actuators is a time-dependent value and wherein said thermal transport property is thermal conductivity, thermal diffusivity or heat capacity per unit volume.

86. (canceled)

87. The method of claim 86 further comprising determining one or more tissue parameters selected from the group consisting of hydration state, stratum corneum thickness, epidermis thickness and vasculature structure using said thermal transport property.

88-89. (canceled)

90. The method of claim 83, wherein said step of determining an effective distance of said one or more thermal sensors from said one or more thermal actuators comprises subtracting a response of the thermal sensor furthest from the thermal actuator from that of each of the thermal sensors in the device to minimize effects of fluctuations in ambient temperature.

91. The method of claim 83, further comprising using Eq. (1) to determine a thermal transport property of the tissue
$$T = {T}_{∞} + {A}_{1} \frac{Q}{2 π {A}_{2} {k}_{skin}} erfc ( \frac{{A}_{2} \sqrt{{ρ}_{skin} {c}_{p , skin}}}{\sqrt{4 {k}_{skin} t}} ) \qquad ( 1 )$$
where T_{∞} is the temperature before heating, Q is the heating power, kₛₖᵢₙ is the thermal conductivity of the skin, ρₛₖᵢₙc_{p,skin} is the volumetric heat capacity of skin, t is time, erfc is the complementary error function, A₂ represents the effective distance from the thermal actuator, and A₁ is a parameter that accounts for details associated with the multilayered geometry of the device, or further comprising using Eq. (3) to determine a thermal transport property of the tissue
$$T = {T}_{∞} + {A}_{1} \frac{Q}{2 π r ( t ) {k}_{skin}} erfc ( \frac{r ( t ) \sqrt{{ρ}_{skin} {c}_{p , skin}}}{\sqrt{4 {k}_{skin} t}} ) \qquad ( 3 )$$
where T_{∞} is the temperature before heating, Q is the heating power, kₛₖᵢₙ is the thermal conductivity of the skin, ρₛₖᵢₙc_{p,skin} is the volumetric heat capacity of skin, t is time, erfc is the complementary error function, A₁ is a parameter that accounts for details associated with the multilayered geometry of the device, and r(t) represents the effective distance of the thermal sensor from the thermal actuator.

92-93. (canceled)
